(19) United States
(12) Patent Application Publication
(10) Pub. No.: US 2024/0327400 A1
(43) Pub. Date: Oct. 3, 2024

(54) COMPOSITIONS AND METHODS FOR MODULATING HAIR GROWTH

(71) Applicants: The Regents of the University Of California, Oakland, CA (US); Pelage Pharmaceuticals, Inc., Los Angeles, CA (US)

(72) Inventors: Daniel L. Sun, Los Angeles, CA (US); Michael E. Jung, Los Angeles, CA (US); Daniel Gil, Los Angeles, CA (US); William E. Lowry, Los Angeles, CA (US); Heather R. Christofk, Los Angeles, CA (US); Aimee Flores, Los Angeles, CA (US); Xiaoguang Liu, Santa Monica, CA (US)

(21) Appl. No.: 18/013,808

(22) PCT Filed: Jun. 29, 2021
(86) PCT No.: PCT/US2021/039501

§ 371 Date: Dec. 29, 2022

Related U.S. Application Data
(60) Provisional application No. 63/048,429, filed on Jul. 6, 2020.
(60) Provisional application No. 63/046,629, filed on Jun. 30, 2020.

(51) Int. Cl. C07D 471/04; A61P 17/14

(52) U.S. Cl. CPC C07D 471/04; A61P 17/14

(57) ABSTRACT

The present disclosure relates to novel compounds that are capable of inhibiting the mitochondrial pyruvate carrier and promoting hair growth. The disclosure further relates to methods of promoting hair growth or treating conditions or disorders affecting hair growth, such as baldness or alopecia.

### CROSS-REFERENCE TO RELATED APPLICATIONS

This application claims the benefit of U.S. Provisional Patent Application No. 63/046,629, filed Jun. 30, 2020 and U.S. Provisional Patent Application No. 63/048,429, filed Jul. 6, 2020, both of which are incorporated by reference herein in their entireties.

### BACKGROUND

Hair follicle stem cells (HFSCs) undergo successive rounds of quiescence (telogen) punctuated by brief periods of proliferation correlating with the start of the hair cycle (telogen-anagen transition). Proliferation or activation of HFSCs is well known to be a prerequisite for advancement of the hair cycle. Despite advances in treatment options, baldness and alopecia continue to be conditions that cannot be successfully treated in many individuals. Some of the existing treatments are inconvenient for users, others require surgical intervention or other invasive procedures. Additional therapies are needed.

### SUMMARY

Described herein compounds of Formula 1:

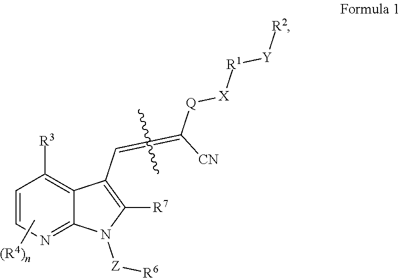

or a pharmaceutically acceptable salt thereof; wherein Q is —C(O)—, —C(═S)—, —S(═O)₂—, —C(═NR⁵)—, —(C═N⁺R⁵R⁸); R¹ is —, —S(═O)₂—, an optionally substituted C₁₋₁₂ hydrocarbon group or an optionally substituted heterocycle; R² is H, an optionally substituted C₁₋₆ alkyl, an optionally substituted carbocycle, or an optionally substituted heterocycle; R³ and R¹ are independently H, F, Cl, Br, I, OH, OR^{A}, SH, SR^{A}, NR^{A}R^{B}, CF₃, CN, carboxylic acid, an optionally substituted carboxylic ester, or an optionally substituted C₁₋₆ alkyl; each R⁴ is independently H, F, Cl, Br, I, OH, O⁺³¹, OR^{A}, SH, SR^{A}, NR^{A}R^{B}, CF₃, CN, carboxylic acid, an optionally substituted carboxylic ester, or an optionally substituted C₁₋₆ alkyl; R^{A} and R^{B} are independently H or optionally substituted C₁₋₆ hydrocarbon group; n is 0, 1, or 2; X is O, S, NR⁵, or N⁺R⁵R⁸; R⁵ and R⁸ are independently H, C₁₋₆ alkyl, an optionally substituted carbocycle, or an optionally substituted heterocycle, and the N, R⁵ and R¹, or the N, R⁵ and R⁸, may together form an optionally substituted heterocyclic ring;
Y is —, —O—,

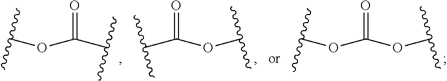

Z is —, an optionally substituted —C(═O)-alkyl, an optionally substituted C₁₋₁₂ hydrocarbon group, or optionally substituted heterocycle; R⁶ is H, an optionally substituted C₁₋₁₂ hydrocarbon group optionally substituted heterocycle; and the wavy line across the C═C bond represents an E or Z olefin.

Some embodiments include a pharmaceutical composition comprising a compound described herein.

Some embodiments include a method of growing hair, comprising: administering a compound described herein to the skin of a mammal, including a human being, in the area where hair growth is intended.

Some embodiments include a method of growing hair comprising administering an MPO inhibitor to a mammal, including a human being, in need thereof. In some embodiments, the MPO inhibitor is a compound described herein.

Some embodiments include a method of treating a disorder affecting hair growth comprising administering a compound described herein to a mammal, including a human being, in need thereof. In some embodiments, the disorder is alopecia or baldness. Some embodiments include use of a compound described herein in the manufacture of a medicament for growing hair.

Some embodiments include a kit comprising a compound described herein and a label with instructions to administer the compound for a use described herein, such as growing hair.

In certain aspects, the present disclosure provides a pharmaceutical composition comprising a compound of the present disclosure and a pharmaceutically acceptable excipient.

In certain aspects, the present disclosure provides methods of enhancing lactate production in a cell, comprising contacting the cell with a compound or composition of the disclosure.

In certain aspects, the present disclosure provides methods of promoting hair growth or treating a hair growth condition or disorder such as baldness or alopecia, comprising administering to a patient a compound of the present disclosure.

### BRIEF DESCRIPTION OF THE DRAWINGS

FIG. 1 shows the conversion of ester prodrug (shown in gray) to the corresponding carboxylic acid API (shown in black) after 1 hour of incubation in homogenized mouse skin at 37° C. and pH 7.4.

FIG. 2 shows the conversion of ester prodrug (shown in gray) to the corresponding carboxylic acid API (shown in black) after 1 hour of incubation in homogenized minipig skin at 37° C. and pH 7.4.

FIGS. 3A and 3B show the conversion of ester prodrug (shown in gray) to the corresponding carboxylic acid API (shown in black) after 1 hour of incubation in homogenized human skin at 37° C. and pH 7.4.

FIG. 4 shows a schematic for performing the LDH activity assay on human skin cell lysate.

FIG. 5A shows that pretreatment of human skin lysate with high heat kills the LDH activity.

FIG. 5B shows that treatment of human skin lysate with exemplary LDH inhibitors blocks most of the LDH activity, further confirming that the activity readout is the result of LDH activity.

FIGS. 6A and 6B, show that the treatment of human skin lysate with exemplary MPC inhibitors results in an increase in LDH activity.

FIG. 7 shows that pretreatment of human skin lysate with a carboxylesterase inhibitor (ben) prior to incubation with MPC inhibitors blocks the effect of most of the exemplary ester containing MPC inhibitors; however, the pretreatment had no effect on carboxylic containing MPC inhibitors.

FIG. 8 shows that the MPC inhibitors of the disclosure promote hair growth. Mice were shaved at day 50 when the hair cycle is dormant. Exemplary compounds were applied topically to the shaved area. Macroscopic observation led to the quantification of hair cycle staging shown where the two ester-MPC inhibitors accelerated the hair cycle compared to vehicle control.

### DETAILED DESCRIPTION

Described herein are compounds, compositions, and methods for modulating hair growth. Compounds of the present disclosure include substituted 7-azaindole compounds which may be useful for modulating hair growth.

Unless otherwise indicated, any reference to a compound herein by structure, name, or any other means, includes pharmaceutically acceptable salts, such as sodium, potassium, and ammonium salts; prodrugs, such as ester prodrugs; alternate solid forms, such as polymorphs, solvates, hydrates, etc.; deuterium-modified forms; Z and E olefin isomers; tautomers; or any other chemical species that may rapidly convert to a compound described herein under conditions in which the compounds are used as described herein. In some embodiments, the compound contains more than a natural abundance of deuterium. In some embodiments, one or more of the hydrogen atoms on the compound is replaced by deuterium so that the compound is at least 50%, at least 80%, at least 90%, at least 95%, or at least 99% deuterium in that position.

Examples of pharmaceutically acceptable salts of compounds include, but are not limited to, alkyl, dialkyl, trialkyl or tetra-alkyl ammonium salts. In certain embodiments, contemplated salts described herein include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts described herein include, but are not limited to, Na, Ca, K, Mg, Zn or other metal salts. In certain embodiments, contemplated salts described herein include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, l-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Unless otherwise indicated, when a compound or chemical structural feature (such as alkyl or aryl) is referred to as being “optionally substituted,” it includes a feature that has no substituents (i.e. unsubstituted), or a feature that is “substituted,” meaning that the feature has one or more substituents. The term “substituent” has the broadest meaning known to one of ordinary skill in the art, and includes a moiety that occupies a position normally occupied by one or more hydrogen atoms attached to a parent compound or structural feature. In some embodiments, a substituent may be an ordinary organic moiety known in the art, which may have a molecular weight (e.g. the sum of the atomic masses of the atoms of the substituent) of about 15 g/mol to about 50 g/mol, about 15 g/mol to about 100 g/mol, about 15 g/mol to about 150 g/mol, about 15 g/mol to about 200 g/mol, about 15 g/mol to about 300 g/mol, or about 15 g/mol to about 500 g/mol. In some embodiments, a substituent comprises, or consists of: 0-30, 0-20, 0-10, or 0-5 carbon atoms; and 0-30, 0-20, 0-10, or 0-5 heteroatoms, wherein each heteroatom may independently be: N, O, S, P, Si, F, Cl, Br, or I; provided that the substituent includes one C, N, O, S, P, Si, F, Cl, Br, or I atom. Examples of substituents include, but are not limited to, compounds represented by an empirical formula: C₁₋₁₂H₃₋₂₉O₀₋₄N₀₋₄S₀₋₄F₀₋₂₅Cl₀₋₅Si₀₋₃P₀₋₃, C₀₋₁₂H₀₋₂₉O₁₋₄N₀₋₄S₀₋₄F₀₋₂₅Cl₀₋₅Si₀₋₃P₀₋₃, C₀₋₁₂H₀₋₂₉O₀₋₄N₁₋₄S₀₋₄F₀₋₂₅Cl₀₋₅Si₀₋₃P₀₋₃, C₀₋₁₂H₀₋₂₉O₀₋₄N₀₋₄S₁₋₄F₀₋₂₅Cl₀₋₅Si₀₋₃P₀₋₃, C₀₋₁₂H₀₋₂₉O₀₋₄N₀₋₄S₀₋₄F₁₋₂₅Cl₀₋₅Si₀₋₃P₀₋₃, C₀₋₁₂H₀₋₂₉O₀₋₄N₀₋₄S₀₋₄F₀₋₂₅Cl₁₋₅Si₀₋₃P₀₋₃, C₀₋₁₂H₀₋₂₉O₀₋₄N₀₋₄S₀₋₄F₀₋₂₅Cl₀₋₅Si₁₋₃P₀₋₃, C₀₋₁₂H₀₋₂₉O₀₋₄N₀₋₄S₀₋₄F₀₋₂₅Cl₀₋₅Si₀₋₃P₁₋₃, C₁₋₆H₃₋₁₆O₀₋₄N₀₋₄S₀₋₄F₀₋₁₃Cl₀₋₃Si₀₋₃P₀₋₃, C₀₋₆H₀₋₁₆O₁₋₄N₀₋₄S₀₋₄F₀₋₁₃Cl₀₋₃Si₀₋₃P₀₋₃, C₀₋₆H₀₋₁₇O₀₋₄N₁₋₄S₀₋₄F₀₋₁₃Cl₀₋₃Si₀₋₃P₀₋₃, C₀₋₆H₀₋₁₇O₀₋₄N₀₋₄S₁₋₄F₀₋₁₃Cl₀₋₃Si₀₋₃P₀₋₃, C₀₋₆H₀₋₁₇O₀₋₄N₀₋₄S₀₋₄F₁₋₁₃Cl₀₋₃Si₀₋₃P₀₋₃, C₀₋₆H₀₋₁₇O₀₋₄N₀₋₄S₀₋₄F₀₋₁₃Cl₁₋₃Si₀₋₃P₀₋₃, C₀₋₆H₀₋₁₇O₀₋₄N₀₋₄S₀₋₄F₀₋₁₃Cl₀₋₃Si₁₋₃P₀₋₃, or C₀₋₆H₀₋₁₇O₀₋₄N₀₋₄S₀₋₄F₀₋₁₃Cl₀₋₃Si₀₋₃P₁₋₃, C₁₋₁₂H₃₋₂₉O₀₋₄N₀₋₄S₀₋₄F₀₋₂₅Cl₀₋₅P₀₋₃, C₁₋₁₂H₃₋₂₇O₀₋₄N₀₋₂S₀₋₂F₀₋₂₅Cl₀₋₅P₀₋₁, C₁₋₁₂H₃₋₂₇O₀₋₄N₀₋₂, C₁₋₁₂H₃₋₂₃O₀₋₄, C₁₋₁₂H₃₋₂₇N₀₋₂, C₁₋₉H₃₋₂₁O₀₋₄N₀₋₂S₀₋₂F₀₋₁₉Cl₀₋₅P₀₋₁, C₁₋₉H₃₋₁₉F₀₋₁₉, C₁₋₉H₃₋₂₁O₀₋₄N₀₋₂, C₁₋₉H₃₋₁₉O₀₋₄, C₁₋₉H₃₋₂₁N₀₋₂, C₁₋₆H₃₋₁₃O₀₋₃N₀₋₂S₀₋₂F₀₋₁₃Cl₀₋₅P₀₋₁, C₁₋₆H₃-13F₀₋₁₃, C₁₋₆H₃₋₁₃O₀₋₄N₀₋₂, C₁₋₆H₃₋₁₃O₀₋₄, C₁₋₆H₃₋₁₅N₀₋₂, C₁₋₃H₃₋₉O₀₋₃N₀₋₂S₀₋₂F₀₋₁₃Cl₀₋₅P₀₋₁, C₁₋₃H₃₋₇F₀₋₇, C₁₋₃H₃₋₉O₀₋₃N₀₋₂, C₁₋₃H₃₋₇O₀₋₃, C₁₋₃H₃₋₉N₀₋₂, F, Cl, Br, I, OH, OR^{A}, SH, SR^{A}, NH₂, NHR^{A}, NR^{A}R^{B}, CF₃, CN, carboxylic acid, optionally substituted carboxylic ester, or optionally substituted C₁₋₆ alkyl, such as optionally substituted branched C₂₋₆ alkyl or optionally substituted linear C₁₋₆ alkyl, including optionally substituted branched or linear C₁₋₃ alkyl (e.g. —CH₃, —C₂H₅, —C₃H₇), optionally substituted branched, linear, or cyclic C₃₋₆ alkyl (e.g. —C₃H₇, —C₄H₉, —C₅H₁₁, —C₆H₁₃, cyclopropyl, cyclobutyl, cyclopentyl, cyclohexyl, etc.), alkenyl, alkynyl, heteroalkyl, heteroalkenyl, heteroalkynyl, aryl, heteroaryl, carbocycle, heterocycle, hydroxy, alkoxy (—O-alkyl), aryloxy, acyl (e.g. —C(═O)-hydrocarbyl, —C(═O)-alkyl or —C(═O)-phenyl), acyloxy (e.g. hydrocarbyl-CO₂—, alkyl-CO₂— or phenyl-CO₂—), alkylcarboxylate, thiol, alkylthio (—S-alkyl), cyano, halo, thiocarbonyl, O-carbamyl, N-carbamyl, O-thiocarbamyl, N-thiocarbamyl, C-amido, N-amido, S-sulfonamido, N-sulfonamido, isocyanato, thiocyanato, isothiocyanato, nitro, silyl, sulfenyl, sulfinyl, sulfonyl, haloalkyl, haloalkoxyl, trihalomethanesulfonyl, trihalomethanesulfonamido, etc.

With respect to any relevant structural representations, R^{A} is H or an optionally substituted C₁₋₆ hydrocarbon group, such as optionally substituted C₁₋₆ alkyl, such as optionally substituted C₁₋₃ alkyl (e.g. methyl, ethyl, propyl, isopropyl), optionally substituted C₃₋₆ alkyl (e.g. propyl, isopropyl, C₄H₉, cyclobutyl, C₅H₁₁, cyclopentyl, C₆H₁₃, cyclohexyl, etc.), optionally substituted C₁₋₆ alkenyl, optionally substituted C₁₋₆ alkynyl, optionally substituted phenyl, etc.

With respect to any relevant structural representations, R^{B} is H or an optionally substituted C₁₋₆ hydrocarbon group, such as optionally substituted C₁₋₆ alkyl, such as optionally substituted C₁₋₃ alkyl (e.g. methyl, ethyl, propyl, isopropyl), optionally substituted C₃₋₆ alkyl (e.g. propyl, isopropyl, C₄H₉, cyclobutyl, C₅H₁₁, cyclopentyl, C₆H₁₃, cyclohexyl, etc.), optionally substituted C₁₋₆ alkenyl, optionally substituted C₁₋₆ alkynyl, optionally substituted phenyl, etc.

For convenience, the term “molecular weight” is used with respect to a moiety or part of a molecule to indicate the sum of the atomic masses of the atoms in the moiety or part of a molecule, even though it may not be a complete molecule.

As used herein, the term “alkyl” has the broadest meaning generally understood in the art, and may include a moiety composed of carbon and hydrogen containing no double or triple bonds. Alkyl may be linear alkyl, branched alkyl, cycloalkyl, or a combination thereof, and in some embodiments, may contain from one to thirty-five carbon atoms. In some embodiments, alkyl may include C₁₋₁₀ linear alkyl, such as methyl (—CH₃), ethyl (—CH₂CH₃), n-propyl (—CH₂CH₂CH₃), n-butyl (—CH₂CH₂CH₂CH₃), n-pentyl (—CH₂CH₂CH₂CH₂CH₃), n-hexyl (—CH₂CH₂CH₂CH₂CH₂CH₃), etc.; C₃₋₁₀ branched alkyl, such as C₃H₇ (e.g. iso-propyl), C₄H₉ (e.g. branched butyl isomers), C₅H₁₁ (e.g. branched pentyl isomers), C₆H₁₃ (e.g. branched hexyl isomers), C₇H₁₅ (e.g. branched heptyl isomers), etc.; C₃₋₁₀ cycloalkyl, such as C₃H₅ (e.g. cyclopropyl), C₄H₇ (e.g. cyclobutyl isomers such as cyclobutyl, methylcyclopropyl, etc.), C₅H₉ (e.g. cyclopentyl isomers such as cyclopentyl, methylcyclobutyl, dimethylcyclopropyl, etc.), C₆H₁₁ (e.g. cyclohexyl isomers), C₇H₁₃ (e.g. cycloheptyl isomers), etc.; C₁₋₁₀ straight-chain alkyl groups; C₁-C₁₀ branched-chain alkyl groups; C₁-C₆ straight-chain alkyl groups; C₁-C₆ branched-chain alkyl groups; C₁-C₄ straight-chain alkyl groups; C₁-C₄ branched-chain alkyl groups; methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The alkyl group may be optionally substituted.

With respect to an optionally substituted moiety such as optionally substituted alkyl, a phrase such as “optionally substituted C₁₋₁₂ alkyl” refers to a C₁₋₁₂ alkyl that may be unsubstituted, or may have 1 or more substituents, and does not limit the number of carbon atoms in any substituent. A phrase such as “C₁₋₁₂ optionally substituted alkyl” refers to unsubstituted C₁₋₁₂ alkyl, or substituted alkyl wherein both the alkyl parent and all substituents have from 1-12 carbon atoms. Similar conventions may be applied to other optionally substituted moieties such as aryl and heteroaryl.

Empirical formulas, such as C₁₋₁₂H₃₋₂₅O₀₋₂N₀₋₂F₀₋₁₂, may be used to describe optionally substituted C₁₋₁₂ alkyl chemical compositions. In some embodiments, additional elements S, Si, P, other halogens, or other heteroatoms may also be included in the empirical formula.

The compounds described herein may have any of the following structural representations:

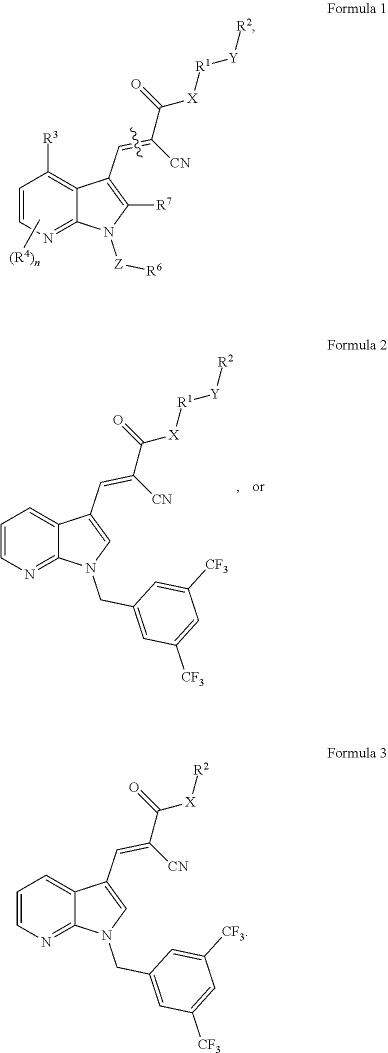

The wavy line across the C═C bond in Formula 1 represents an E or Z olefin, e.g. structures such as Formula 1C and Formula 1 T.

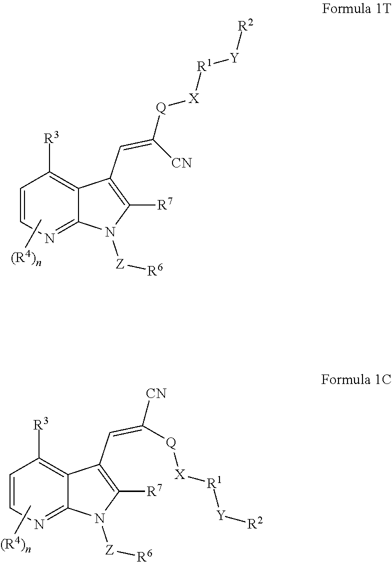

With respect to any relevant structural representation, such as Formula 1, 1T, and 1C, Q is —C(═O)—, —C(═S)—, or —S(═O)₂—. In some embodiments, Q is —C(═O)—. In some embodiments, Q is —C(═S)—. In some embodiments, Q is —S(═O)₂—.

With respect to any relevant structural representation, such as Formula 1, 1C, 1T, or 2, R¹ is a bond (represented as —); —S(═O)₂—; an optionally substituted C₁₋₁₂ hydrocarbon group, including optionally substituted C₁₋₁₂ alkyl, such as optionally substituted branched C₂₋₁₂ alkyl or optionally substituted linear C₁₋₁₂ alkyl, including optionally substituted branched C₂₋₆ alkyl or linear C₁₋₆ alkyl, optionally substituted branched C₂₋₃ alkyl (e.g., —CH(CH₃), —CH(CH₂CH₃), —C(CH₃)₂—), or linear C₁₋₃ alkyl (e.g., —CH₂—, —C₂H₄—, —C₃H₆—), optionally substituted branched, linear, or cyclic C₃₋₆ alkyl (e.g. —C₃H₆—, —C₄H₅—, —C₅H₁₀—, —C₆H₁₂—, —CH(CH₂CH₃), —CH(CH₃)CH₂—, —C(CH₃)₂CH₂—, —CH₂CH(CH₃)CH₂—, —C(CH₃)(CH₂CH₃), —CH(CH₂CH₂CH₃), —C(CH₂CH₃)₂—, —C(CH₃)(CH₂CH₂CH₃), —CH(CH₂CH₂CH₂CH₃)—, —C(CH₃)(CH₂CH₂CH₂CH₃)—, —CH(CH₂CH₂CH₂CH₂CH₃), —C(CH₂CH₃)(CH₂CH₂CH₃)—, cyclopropyl, cyclobutyl, cyclopentyl, cyclohexyl, etc.), optionally substituted branched, linear, or cyclic C₆₋₉ alkyl (e.g., —C(CH₃)(CH₂CH₂CH₂CH₃)—, —CH(CH₂CH₂CH₂CH₂CH₃)—, —C(CH₂CH₃)(CH₂CH₂CH₃), —C₆H₁₂—, —C₇H₁₄—, —C₈H₁₆—, —C₉H₁₈—, cyclohexyl, cycloheptyl, cyclooctyl, cyclononyl, etc.), optionally substituted branched, linear, or cyclic C₉₋₁₂ alkyl, C₂₋₁₂ alkenyl, C₂₋₁₂ alkynyl, optionally substituted C₃₋₁₂ carbocycle, optionally substituted benzyl, etc.; optionally substituted carbocycle, including optionally substituted C₃₋₁₂ cycloalkyl, optionally substituted C₃₋₆ cycloalkyl, optionally substituted C₆₋₉ cycloalkyl, optionally substituted C₉₋₁₂ cycloalkyl, optionally substituted C₃₋₁₂ cycloalkenyl, optionally substituted C₃₋₆ cycloalkenyl, optionally substituted C₆₋₉ cycloalkenyl, optionally substituted C₉₋₁₂ cycloalkenyl, optionally substituted C₃₋₁₂ cycloalkynyl, optionally substituted C₃₋₆ cycloalkynyl, optionally substituted C₆₋₉ cycloalkynyl, optionally substituted C₉₋₁₂ cycloalkynyl, optionally substituted phenyl, optionally substituted naphthyl; or optionally substituted heterocycle, such as an optionally substituted monocyclic heterocycle having 3 ring carbon atoms and 1 ring oxygen atom, an optionally substituted monocyclic heterocycle having 4 ring carbon atoms and 1 ring oxygen atom, an optionally substituted monocyclic heterocycle having 5 ring carbon atoms and 1 ring oxygen atom, an optionally substituted monocyclic heterocycle having 6 ring carbon atoms and 1 ring oxygen atom, an optionally substituted monocyclic heterocycle having 7 ring carbon atoms and 1 ring oxygen atom, an optionally substituted monocyclic heterocycle having 3 ring carbon atoms and 1 ring sulfur atom, an optionally substituted monocyclic heterocycle having 4 ring carbon atoms and 1 ring sulfur atom, an optionally substituted monocyclic heterocycle having 5 ring carbon atoms and 1 ring sulfur atom, an optionally substituted monocyclic heterocycle having 6 ring carbon atoms and 1 ring sulfur atom, an optionally substituted monocyclic heterocycle having 7 ring carbon atoms and 1 ring sulfur atom, an optionally substituted monocyclic heterocycle having 3 ring carbon atoms and 1 ring nitrogen atom, an optionally substituted monocyclic heterocycle having 4 ring carbon atoms and 1 ring nitrogen atom, an optionally substituted monocyclic heterocycle having 5 ring carbon atoms and 1 ring nitrogen atom, an optionally substituted monocyclic heterocycle having 6 ring carbon atoms and 1 ring nitrogen atom, an optionally substituted monocyclic heterocycle having 7 ring carbon atoms and 1 ring nitrogen atom, an optionally substituted monocyclic heterocycle having 3 ring carbon atoms and 2 ring heteroatoms (N, O, and/or S), an optionally substituted monocyclic heterocycle having 4 ring carbon atoms and 2 ring heteroatoms (N, O, and/or S), an optionally substituted monocyclic heterocycle having 5 ring carbon atoms and 2 ring heteroatoms (N, O, and/or S), an optionally substituted monocyclic heterocycle having 6 ring carbon atoms and 2 ring heteroatoms (N, O, and/or S), an optionally substituted monocyclic heterocycle having 2 ring carbon atoms and 3 ring heteroatoms (N, O, and/or S), an optionally substituted monocyclic heterocycle having 3 ring carbon atoms and 3 ring heteroatoms (N, O, and/or S), an optionally substituted monocyclic heterocycle having 4 ring carbon atoms and 3 ring heteroatoms (N, O, and/or S), an optionally substituted monocyclic heterocycle having 5 ring carbon atoms and 3 ring heteroatoms (N, O, and/or S), an optionally substituted bicyclic heterocycle having 6 ring carbon atoms and 1 ring heteroatom (N, O, or S), an optionally substituted bicyclic heterocycle having 7 ring carbon atoms and 1 ring heteroatom (N, O, or S), an optionally substituted bicyclic heterocycle having 8 ring carbon atoms and 1 ring heteroatom (N, O, or S), an optionally substituted bicyclic heterocycle having 9 ring carbon atoms and 1 ring heteroatom (N, O, or S), an optionally substituted bicyclic heterocycle having 10 ring carbon atoms and 1 ring heteroatom (N, O, or S), an optionally substituted bicyclic heterocycle having 11 ring carbon atoms and 1 ring heteroatom (N, O, or S), an optionally substituted bicyclic heterocycle having 12 ring carbon atoms and 1 ring heteroatom (N, O, or S), an optionally substituted bicyclic heterocycle having 5 ring carbon atoms and 2 ring heteroatoms (N, O, and/or S), an optionally substituted bicyclic heterocycle having 6 ring carbon atoms and 2 ring heteroatoms (N, O, and/or S), an optionally substituted bicyclic heterocycle having 7 ring carbon atoms and 2 ring heteroatoms (N, O, and/or S), an optionally substituted bicyclic heterocycle having 8 ring carbon atoms and 2 ring heteroatoms (N, O, and/or S), an optionally substituted bicyclic heterocycle having 9 ring carbon atoms and 2 ring heteroatoms (N, O, and/or S), an optionally substituted bicyclic heterocycle having 10 ring carbon atoms and 2 ring heteroatoms (N, O, and/or S), an optionally substituted bicyclic heterocycle having 11 ring carbon atoms and 2 ring heteroatoms (N, O, and/or S), an optionally substituted bicyclic heterocycle having 5 ring carbon atoms and 3 ring heteroatoms (N, O, and/or S), an optionally substituted bicyclic heterocycle having 6 ring carbon atoms and 3 ring heteroatoms (N, O, and/or S), an optionally substituted bicyclic heterocycle having 7 ring carbon atoms and 3 ring heteroatoms (N, O, and/or S), an optionally substituted bicyclic heterocycle having 8 ring carbon atoms and 3 ring heteroatoms (N, O, and/or S), an optionally substituted bicyclic heterocycle having 9 ring carbon atoms and 3 ring heteroatoms (N, O, and/or S), an optionally substituted bicyclic heterocycle having 10 ring carbon atoms and 3 ring heteroatoms (N, O, and/or S), optionally substituted oxetane, optionally substituted tetrahydrofuran, optionally substituted dihydrofuran, optionally substituted furan, optionally substituted furanone, optionally substituted tetrahydropyran, optionally substituted dihydropyran, an optionally substituted pyran, optionally substituted tetrahydropyrone, optionally substituted dihydropyrone, optionally substituted pyrone, optionally substituted thietane, optionally substituted tetrahydrothiophene, optionally substituted dihydrothiophene, an optionally substituted thiophene, optionally substituted azetidine, optionally substituted pyrrolidine, optionally substituted pyrroline, optionally substituted pyrrole, optionally substituted piperidine, optionally substituted pyridine, optionally substituted oxazole, optionally substituted isoxazole, optionally substituted thiazole, optionally substituted isothiazole, optionally substituted pyrazolidine, optionally substituted imidazolidine, optionally substituted pyrazole, optionally substituted imidazole, optionally substituted tetrazole, optionally substituted sulfolane. When X is NR⁵, the N, R¹ and R⁵ may together form an optionally substituted heterocyclic ring (such as optionally substituted morpholine). When X is N⁺R¹R⁵, the N, R⁵ and R¹, or the N, R⁵ and R⁸, may together form an optionally substituted heterocyclic ring (such as optionally substituted morpholinium).

For the purposes of this disclosure, the term “alkyl” refers to both monovalent groups (such as —CH₃), bivalent groups (such as —CH₂—), or other hydrocarbon groups with higher valency that are free of double and triple bonds.

In some embodiments, R¹ is —. In some embodiments, R¹ is C₁₋₁₂ alkyl. In some embodiments, R¹ is linear C₁₋₁₂ alkyl. In some embodiments, R¹ is branched C₂₋₁₂ alkyl. In some embodiments, R¹ is —CH₂—, —C₂H₄—, —C₃H₆—, —C₃H₆—, —C₄H₅—, —C₅H₁₀—, —C₆H₁₂—, —C₇H₁₄—, —C₈H₁₆—, or —C₉H₁₈—. In some embodiments, R¹ is —CH₂—. In some embodiments, R¹ is —C₂H₄—. In some embodiments, R¹ is —C₃H₆—. In some embodiments, R¹ is —C₃H₆—. In some embodiments, R¹ is —C₄H₅—. In some embodiments, R¹ is —C₅H₁₀—. In some embodiments, R¹ is —C₆H₁₂—. In some embodiments, R¹ is —C₇H₁₄—. In some embodiments, R¹ is —C₂H₁₆—. In some embodiments, R¹ is —C₉H₁₈—. In some embodiments, R¹ is an optionally substituted linear C₁₋₁₂ alkyl. In some embodiments, R¹ is an optionally substituted branched C₂₋₁₂ alkyl. In some embodiments, R¹ is an optionally heteroatom substituted branched C₂₋₁₂ alkyl, such as a branched C₂₋₁₂ alkyl having polar substituents, including oxygen containing groups (e.g. —OH, =O, OCH₃, etc.), sulfur containing groups (e.g. —SH, —SCH₃, SO₂, SO₃⁻, etc.), nitrogen containing groups (e.g. amino groups such as —NH₂, —NHCH₃, —N(CH₃)₂, quaternary ammonium salts such as —[N(CH₃)₂]⁺, —[N(CH₂CH₃)(CH₃)]⁺, —NO₂, —CN, etc.), fluorine containing groups (e.g. F, CF₃, CF₂CF₃, CHF₂, CH₂F, CF₂CF₂CF₃, etc.).

In some embodiments, R¹ is an optionally substituted carbocycle. In some embodiments, R¹ is optionally substituted cyclohexyl. In some embodiments, R¹ is an optionally substituted aryl. In some embodiments, R¹ is an optionally substituted phenyl. In some embodiments, R¹ is an optionally substituted benzyl. In some embodiments, R¹ is an optionally substituted heteroaryl. In some embodiments, R¹ is an optionally substituted heterocycle. In some embodiments wherein R¹ is an optionally substituted heterocycle, a carbon atom of the heterocycle (rather than a heteroatom of the heterocycle) is directly attached to X. In some embodiments wherein R¹ is an optionally substituted heterocycle, a carbon atom of the heterocycle ring (rather than a heteroatom of the heterocycle ring) is directly attached to Y. In some embodiments, R¹ is an optionally heteroatom substituted carbocycle, such as a carbocycle having polar substituents, including oxygen containing groups (e.g. —OH, =O, OCH₃, etc.), sulfur containing groups (e.g. —SH, —SCH₃, SO₂, SO₃⁻, etc.), nitrogen containing groups (e.g. amino groups such as —NH₂, —NHCH₃, —N(CH₃)₂, quaternary ammonium salts such as —[N(CH₃)₂]⁺, —[N(CH₂CH₃)(CH₃)]⁺, —NO₂, —CN, etc.), fluorine containing groups (e.g. F, CF₃, CF₂CF₃, CHF₂, CH₂F, CF₂CF₂CF₃, etc.). In some embodiments, R¹ is an optionally heteroatom substituted heterocycle, such as a heterocycle having polar substituents, including oxygen containing groups (e.g. —OH, =O, OCH₃, etc.), sulfur containing groups (e.g. —SH, —SCH₃, SO₂, SO₃⁻, etc.), nitrogen containing groups (e.g. —NH₂, —NHCH₃, —N(CH₃)₂, —NO₂, —CN, etc.), fluorine containing groups (F, CF₃, CF₂CF₃, CHF₂, CH₂F, CF₂CF₂CF₃, etc.). In some embodiments, R¹ is an optionally heteroatom substituted benzyl, such as a benzyl having polar substituents, including oxygen containing groups (e.g. —OH, =O, OCH₃, etc.), sulfur containing groups (e.g. —SH, —SCH₃, SO₂, SO₃⁻, etc.), nitrogen containing groups (e.g. —NH₂, —NHCH₃, —N(CH₃)₂, —NO₂, —CN, etc.), fluorine containing groups (e.g. F, CF₃, CF₂CF₃, CHF₂, CH₂F, CF₂CF₂CF₃, etc.).

In some embodiments, R¹ is an optionally substituted oxetane. In some embodiments, R¹ is an optionally substituted oxetane having a carbon atom of the oxetane ring directly attached to X.

In some embodiments, R¹ is an optionally substituted tetrahydrofuran. In some embodiments, R¹ is an optionally substituted tetrahydrofuran having a carbon atom of the tetrahydrofuran ring directly attached to X.

In some embodiments, R¹ is an optionally substituted dihydrofuran. In some embodiments, R¹ is an optionally substituted dihydrofuran having a carbon atom of the dihydrofuran ring directly attached to X.

In some embodiments, R¹ is an optionally substituted furan. In some embodiments, R¹ is an optionally substituted furan having a carbon atom of the furan ring directly attached to X.

In some embodiments, R¹ is an optionally substituted furanone. In some embodiments, R¹ is an optionally substituted furanone having a carbon atom of the furanone ring directly attached to X.

In some embodiments, R¹ is an optionally substituted tetrahydropyran. In some embodiments, R¹ is an optionally substituted tetrahydropyran having a carbon atom of the tetrahydropyran ring directly attached to X.

In some embodiments, R¹ is an optionally substituted dihydropyran. In some embodiments, R¹ is an optionally substituted dihydropyran having a carbon atom of the dihydropyran ring directly attached to X.

In some embodiments, R¹ is an optionally substituted pyran. In some embodiments, R¹ is an optionally substituted pyran having a carbon atom of the pyran ring directly attached to X.

In some embodiments, R¹ is an optionally substituted tetrahydropyrone. In some embodiments, R¹ is an optionally substituted tetrahydropyrone having a carbon atom of the tetrahydropyrone ring directly attached to X.

In some embodiments, R¹ is an optionally substituted dihydropyrone. In some embodiments, R¹ is an optionally substituted dihydropyrone having a carbon atom of the dihydropyrone ring directly attached to X.

In some embodiments, R¹ is an optionally substituted pyrone. In some embodiments, R¹ is an optionally substituted pyrone having a carbon atom of the pyrone ring directly attached to X.

In some embodiments, R¹ is an optionally substituted thietane. In some embodiments, R¹ is an optionally substituted thietane having a carbon atom of the thietane ring directly attached to X.

In some embodiments, R¹ is an optionally substituted tetrahydrothiophene. In some embodiments, R¹ is an optionally substituted tetrahydrothiophene having a carbon atom of the tetrahydrothiophene ring directly attached to X.

In some embodiments, R¹ is an optionally substituted dihydrothiophene. In some embodiments, R¹ is an optionally substituted dihydrothiophene having a carbon atom of the dihydrothiophene ring directly attached to X.

In some embodiments, R¹ is an optionally substituted thiophene. In some embodiments, R¹ is an optionally substituted thiophene having a carbon atom of the thiophene ring directly attached to X.

In some embodiments, R¹ is an optionally substituted azetidine. In some embodiments, R¹ is an optionally substituted azetidine having a carbon atom of the azetidine ring directly attached to X. In some embodiments, R¹ is azetidine having an optionally substituted diphenylmethyl substituent. In some embodiments, R¹ is azetidine having an optionally substituted diphenylmethyl substituent attached to the nitrogen atom of the azetidine ring.

In some embodiments, R¹ is an optionally substituted pyrrolidine. In some embodiments, R¹ is an optionally substituted pyrrolidine having a carbon atom of the pyrrolidine ring directly attached to X.

In some embodiments, R¹ is an optionally substituted pyrroline. In some embodiments, R¹ is an optionally substituted pyrroline having a carbon atom of the pyrroline ring directly attached to X.

In some embodiments, R¹ is an optionally substituted pyrrole. In some embodiments, R¹ is an optionally substituted pyrrole having a carbon atom of the pyrrole ring directly attached to X.

In some embodiments, R¹ is an optionally substituted piperidine. In some embodiments, R¹ is an optionally substituted piperidine having a carbon atom of the piperidine ring directly attached to X.

In some embodiments, R¹ is an optionally substituted pyridine. In some embodiments, R¹ is an optionally substituted pyridine having a carbon atom of the pyridine ring directly attached to X.

In some embodiments, R¹ is an optionally substituted oxazole. In some embodiments, R¹ is an optionally substituted oxazole having a carbon atom of the oxazole ring directly attached to X.

In some embodiments, R¹ is an optionally substituted isoxazole. In some embodiments, R¹ is an optionally substituted isoxazole having a carbon atom of the isoxazole ring directly attached to X.

In some embodiments, R¹ is an optionally substituted thiazole. In some embodiments, R¹ is an optionally substituted thiazole having a carbon atom of the thiazole ring directly attached to X.

In some embodiments, R¹ is an optionally substituted isothiazole. In some embodiments, R¹ is an optionally substituted isothiazole having a carbon atom of the isothiazole ring directly attached to X.

In some embodiments, R¹ is an optionally substituted pyrazolidine. In some embodiments, R¹ is an optionally substituted pyrazolidine having a carbon atom of the pyrazolidine ring directly attached to X.

In some embodiments, R¹ is an optionally substituted imidazolidine. In some embodiments, R¹ is an optionally substituted imidazolidine having a carbon atom of the imidazolidine ring directly attached to X.

In some embodiments, R¹ is an optionally substituted pyrazole. In some embodiments, R¹ is an optionally substituted pyrazole having a carbon atom of the pyrazole ring directly attached to X.

In some embodiments, R¹ is an optionally substituted imidazole. In some embodiments, R¹ is an optionally substituted imidazole having a carbon atom of the imidazole ring directly attached to X.

In some embodiments, R¹ is an optionally substituted tetrazole. In some embodiments, R¹ is an optionally substituted tetrazole having a carbon atom of the tetrazole ring directly attached to X.

In some embodiments, R¹ is an optionally substituted sulfolane. In some embodiments, R¹ is an optionally substituted sulfolane having a carbon atom of the sulfolane ring directly attached to X.

In some embodiments, R¹ is —S(O)₂—.

In some embodiments, R¹ is —CH₂—, —CH₂CH(CH₃)CH₂—, or oxetane having a carbon atom of the oxetane ring directly attached to X. In some embodiments, R¹ is —CH₂—. In some embodiments, R¹ is —CH₂CH(CH₃)CH₂—. In some embodiments, R¹ is oxetane having a carbon atom of the oxetane ring directly attached to X.

In some embodiments, for a compound of Formula 1, 1C, or 1T, R¹ is —, —CH₂—, an optionally substituted C₃₋₁₂ hydrocarbon group, or an optionally substituted heterocycle having a carbon atom directly attached to X.

In some embodiments, for a compound of Formula 2, R¹ is —, —CH₂—, an optionally substituted C₃₋₁₂ hydrocarbon group, or an optionally substituted heterocycle having a carbon atom directly attached to X.

With respect to any relevant structural representation, such as Formula 1, 1C, 1T, 2, or 3, R² is H; optionally substituted C₁₋₁₂ alkyl, optionally substituted C₁₋₆ alkyl group, such as optionally substituted branched C₃₋₆ alkyl or linear C₁₋₆ alkyl, optionally substituted branched C₃ alkyl (e.g., —CH(CH₃)₂), or optionally substituted linear C₁₋₃ alkyl (e.g., —CH₃, —C₂H₅, —C₃H₇), optionally substituted branched, linear, or cyclic C₃₋₆ alkyl (e.g. —C₃H₇, —C₄H₉, —C₅H₁₁, —C₆H₁₃, —CH(CH₃)₂, —CH(CH₃)(CH₂CH₃), —C(CH₃)₃, —CH(CH₂CH₃)₂, —CH(CH₃)(CH₂CH₂CH₃), —C(CH₃)₂(CH₂CH₃), —CH₂CH₂CH₂CH(CH₃)₂, —CH₂CH(CH₃)CH₂CH₂CH₃, —CH₂CH₂CH(CH₃)CH₂CH₃, —CH(CH₂CH₃)(CH₂CH₂CH₃), —C(CH₃)(CH₂CH₃)₂, cycloalkyl, including cyclopropyl, cyclobutyl, cyclopentyl, cyclohexyl, etc.); optionally substituted carbocycle, including optionally substituted cycloalkyl, such as optionally substituted C₃₋₆ cycloalkyl, optionally substituted C₃₋₆ cycloalkenyl, optionally substituted C₃₋₆ cycloalkynyl, optionally substituted phenyl; or optionally substituted heterocycle such as an optionally substituted monocyclic heterocycle having 3 ring carbon atoms and 1 ring oxygen atom, an optionally substituted monocyclic heterocycle having 4 ring carbon atoms and 1 ring oxygen atom, an optionally substituted monocyclic heterocycle having 5 ring carbon atoms and 1 ring oxygen atom, an optionally substituted monocyclic heterocycle having 6 ring carbon atoms and 1 ring oxygen atom, an optionally substituted monocyclic heterocycle having 7 ring carbon atoms and 1 ring oxygen atom, an optionally substituted monocyclic heterocycle having 3 ring carbon atoms and 1 ring sulfur atom, an optionally substituted monocyclic heterocycle having 4 ring carbon atoms and 1 ring sulfur atom, an optionally substituted monocyclic heterocycle having 5 ring carbon atoms and 1 ring sulfur atom, an optionally substituted monocyclic heterocycle having 6 ring carbon atoms and 1 ring sulfur atom, an optionally substituted monocyclic heterocycle having 7 ring carbon atoms and 1 ring sulfur atom, an optionally substituted monocyclic heterocycle having 3 ring carbon atoms and 1 ring nitrogen atom, an optionally substituted monocyclic heterocycle having 4 ring carbon atoms and 1 ring nitrogen atom, an optionally substituted monocyclic heterocycle having 5 ring carbon atoms and 1 ring nitrogen atom, an optionally substituted monocyclic heterocycle having 6 ring carbon atoms and 1 ring nitrogen atom, an optionally substituted monocyclic heterocycle having 7 ring carbon atoms and 1 ring nitrogen atom, an optionally substituted monocyclic heterocycle having 3 ring carbon atoms and 2 ring heteroatoms (N, O, and/or S), an optionally substituted monocyclic heterocycle having 4 ring carbon atoms and 2 ring heteroatoms (N, O, and/or S), an optionally substituted monocyclic heterocycle having 5 ring carbon atoms and 2 ring heteroatoms (N, O, and/or S), an optionally substituted monocyclic heterocycle having 6 ring carbon atoms and 2 ring heteroatoms (N, O, and/or S), an optionally substituted monocyclic heterocycle having 2 ring carbon atoms and 3 ring heteroatoms (N, O, and/or S), an optionally substituted monocyclic heterocycle having 3 ring carbon atoms and 3 ring heteroatoms (N, O, and/or S), an optionally substituted monocyclic heterocycle having 4 ring carbon atoms and 3 ring heteroatoms (N, O, and/or S), an optionally substituted monocyclic heterocycle having 5 ring carbon atoms and 3 ring heteroatoms (N, O, and/or S), an optionally substituted bicyclic heterocycle having 6 ring carbon atoms and 1 ring heteroatom (N, O, or S), an optionally substituted bicyclic heterocycle having 7 ring carbon atoms and 1 ring heteroatom (N, O, or S), an optionally substituted bicyclic heterocycle having 8 ring carbon atoms and 1 ring heteroatom (N, O, or S), an optionally substituted bicyclic heterocycle having 9 ring carbon atoms and 1 ring heteroatom (N, O, or S), an optionally substituted bicyclic heterocycle having 10 ring carbon atoms and 1 ring heteroatom (N, O, or S), an optionally substituted bicyclic heterocycle having 11 ring carbon atoms and 1 ring heteroatom (N, O, or S), an optionally substituted bicyclic heterocycle having 12 ring carbon atoms and 1 ring heteroatom (N, O, or S), an optionally substituted bicyclic heterocycle having 5 ring carbon atoms and 2 ring heteroatoms (N, O, and/or S), an optionally substituted bicyclic heterocycle having 6 ring carbon atoms and 2 ring heteroatoms (N, O, and/or S), an optionally substituted bicyclic heterocycle having 7 ring carbon atoms and 2 ring heteroatoms (N, O, and/or S), an optionally substituted bicyclic heterocycle having 8 ring carbon atoms and 2 ring heteroatoms (N, O, and/or S), an optionally substituted bicyclic heterocycle having 9 ring carbon atoms and 2 ring heteroatoms (N, O, and/or S), an optionally substituted bicyclic heterocycle having 10 ring carbon atoms and 2 ring heteroatoms (N, O, and/or S), an optionally substituted bicyclic heterocycle having 11 ring carbon atoms and 2 ring heteroatoms (N, O, and/or S), an optionally substituted bicyclic heterocycle having 5 ring carbon atoms and 3 ring heteroatoms (N, O, and/or S), an optionally substituted bicyclic heterocycle having 6 ring carbon atoms and 3 ring heteroatoms (N, O, and/or S), an optionally substituted bicyclic heterocycle having 7 ring carbon atoms and 3 ring heteroatoms (N, O, and/or S), an optionally substituted bicyclic heterocycle having 8 ring carbon atoms and 3 ring heteroatoms (N, O, and/or S), an optionally substituted bicyclic heterocycle having 9 ring carbon atoms and 3 ring heteroatoms (N, O, and/or S), an optionally substituted bicyclic heterocycle having 10 ring carbon atoms and 3 ring heteroatoms (N, O, and/or S), optionally substituted oxetane, optionally substituted tetrahydrofuran, optionally substituted dihydrofuran, optionally substituted furan, optionally substituted furanone, optionally substituted tetrahydropyran, optionally substituted dihydropyran, an optionally substituted pyran, optionally substituted tetrahydropyrone, optionally substituted dihydropyrone, optionally substituted pyrone, optionally substituted thietane, optionally substituted tetrahydrothiophene, optionally substituted dihydrothiophene, an optionally substituted thiophene, optionally substituted azetidine, optionally substituted pyrrolidine, optionally substituted pyrroline, optionally substituted pyrrole, optionally substituted piperidine, optionally substituted pyridine, optionally substituted oxazole, optionally substituted isoxazole, optionally substituted thiazole, optionally substituted isothiazole, optionally substituted pyrazolidine, optionally substituted imidazolidine, optionally substituted pyrazole, optionally substituted imidazole, optionally substituted tetrazole, optionally substituted sulfolane.

In some embodiments, R² is H. In some embodiments, R² is C₁₋₆ alkyl. In some embodiments, R² is branched C₂₋₆ alkyl. In some embodiments, R² is —CH₃, —C₂H₅, —C₃H₇, —C₄H₉, —C₅H₁₁, or —C₆H₁₃. In some embodiments, R² is —CH₃. In some embodiments, R² is —C₂H₅. In some embodiments, R² is —C₃H₇. In some embodiments, R² is —C₄H₉. In some embodiments, R² is —C₅H₁₁. In some embodiments, R² is —C₆H₁₃. In some embodiments, R² is an optionally substituted linear C₁₋₆ alkyl. In some embodiments, R² is isopropyl. In some embodiments, R² is isobutyl. In some embodiments, R² is tert-butyl. In some embodiments, R² is fluoro substituted C₁₋₆ alkyl, including C₁₋₆ perfluoralkyl. In some embodiments, R² is fluoro substituted branched C₂₋₆ alkyl, such as branched C₂₋₆ perfluoroalkyl. In some embodiments, R² is —CF₃, —C₂F₅, —C₃F₇, —C₄F₉, —C₅F₁₁, or —C₆F₁₃. In some embodiments, R² is —CF₃. In some embodiments, R² is —C₂F₅. In some embodiments, R² is —C₃F₇. In some embodiments, R² is —C₄F₉. In some embodiments, R² is —C₅F₁₁. In some embodiments, R² is —C₆F₁₃. In some embodiments, R² is CF₃. In some embodiments, R² is CHF₂. In some embodiments, R² is CH₂F. In some embodiments, R² is CF₂CF₃. In some embodiments, R² is CF₂CF₂CF₃. In some embodiments, R² is fluoro substituted isopropyl, including perfluoroisopropyl. In some embodiments, R² is fluoro substituted isobutyl, including perfluoroisobutyl. In some embodiments, R² is fluoro substituted tert-butyl including perfluoro-tert-butyl.

In some embodiments, R² is an optionally substituted carbocycle. In some embodiments, R² is optionally substituted cyclohexyl. In some embodiments, R² is an optionally substituted aryl. In some embodiments, R² is an optionally substituted phenyl. In some embodiments, R² is optionally substituted benzyl. In some embodiments, R² is an optionally substituted heteroaryl. In some embodiments, R² is an optionally substituted heterocycle. In some embodiments wherein R² is an optionally substituted heterocycle, a carbon atom of the heterocycle (rather than a heteroatom of the heterocycle) is directly attached to Y.

In some embodiments, R² is an optionally substituted carbocycle, such as a carbocycle having electron-withdrawing substituents including acyl groups (e.g., —C(O)R, etc.) esters (e.g., —CO₂R, etc.), amides (e.g., —C(O)NR₂, etc.), imides (e.g., —C(O)NRC(O)R, etc.), cyano (—CN), sulfones (e.g., —SO₂R, etc.), sulfonamides (e.g., —SO₂NR₂), fluorine or fluorine containing groups (e.g., F, CF₃, CF₂CF₃, CHF₂, CH₂F, CF₂CF₂CF₃, etc.), and/or nitro (—NO₂). In some aspects, R² is an electron-deficient heterocyclic moiety.

In some embodiments, R² is an optionally substituted oxetane. In some embodiments, R² is an optionally substituted oxetane having a carbon atom of the oxetane ring directly attached to Y.

In some embodiments, R² is an optionally substituted tetrahydrofuran. In some embodiments, R² is an optionally substituted tetrahydrofuran having a carbon atom of the tetrahydrofuran ring directly attached to Y.

In some embodiments, R² is an optionally substituted dihydrofuran. In some embodiments, R² is an optionally substituted dihydrofuran having a carbon atom of the dihydrofuran ring directly attached to Y.

In some embodiments, R² is an optionally substituted furan. In some embodiments, R² is an optionally substituted furan having a carbon atom of the furan ring directly attached to Y.

In some embodiments, R² is an optionally substituted furanone. In some embodiments, R² is an optionally substituted furanone having a carbon atom of the furanone ring directly attached to Y.

In some embodiments, R² is an optionally substituted tetrahydropyran. In some embodiments, R² is an optionally substituted tetrahydropyran having a carbon atom of the tetrahydropyran ring directly attached to Y.

In some embodiments, R² is an optionally substituted dihydropyran. In some embodiments, R² is an optionally substituted dihydropyran having a carbon atom of the dihydropyran ring directly attached to Y.

In some embodiments, R² is an optionally substituted pyran. In some embodiments, R² is an optionally substituted pyran having a carbon atom of the pyran ring directly attached to Y.

In some embodiments, R² is an optionally substituted tetrahydropyrone. In some embodiments, R² is an optionally substituted tetrahydropyrone having a carbon atom of the tetrahydropyrone ring directly attached to Y.

In some embodiments, R² is an optionally substituted dihydropyrone. In some embodiments, R² is an optionally substituted dihydropyrone having a carbon atom of the dihydropyrone ring directly attached to Y.

In some embodiments, R² is an optionally substituted pyrone. In some embodiments, R² is an optionally substituted pyrone having a carbon atom of the pyrone ring directly attached to Y.

In some embodiments, R² is an optionally substituted thietane. In some embodiments, R² is an optionally substituted thietane having a carbon atom of the thietane ring directly attached to Y.

In some embodiments, R² is an optionally substituted tetrahydrothiophene. In some embodiments, R² is an optionally substituted tetrahydrothiophene having a carbon atom of the tetrahydrothiophene ring directly attached to Y.

In some embodiments, R² is an optionally substituted dihydrothiophene. In some embodiments, R² is an optionally substituted dihydrothiophene having a carbon atom of the dihydrothiophene ring directly attached to Y.

In some embodiments, R² is an optionally substituted thiophene. In some embodiments, R² is an optionally substituted thiophene having a carbon atom of the thiophene ring directly attached to Y.

In some embodiments, R² is an optionally substituted azetidine. In some embodiments, R² is an optionally substituted azetidine having a carbon atom of the azetidine ring directly attached to Y. In some embodiments, R² is azetidine having an optionally substituted diphenylmethyl substituent. In some embodiments, R² is azetidine having an optionally substituted diphenylmethyl substituent attached to the nitrogen atom of the azetidine ring.

In some embodiments, R² is an optionally substituted pyrrolidine. In some embodiments, R² is an optionally substituted pyrrolidine having a carbon atom of the pyrrolidine ring directly attached to Y.

In some embodiments, R² is an optionally substituted pyrroline. In some embodiments, R² is an optionally substituted pyrroline having a carbon atom of the pyrroline ring directly attached to Y.

In some embodiments, R² is an optionally substituted pyrrole. In some embodiments, R² is an optionally substituted pyrrole having a carbon atom of the pyrrole ring directly attached to Y.

In some embodiments, R² is an optionally substituted piperidine. In some embodiments, R² is an optionally substituted piperidine having a carbon atom of the piperidine ring directly attached to Y.

In some embodiments, R² is an optionally substituted pyridine. In some embodiments, R² is an optionally substituted pyridine having a carbon atom of the pyridine ring directly attached to Y.

In some embodiments, R² is an optionally substituted oxazole. In some embodiments, R² is an optionally substituted oxazole having a carbon atom of the oxazole ring directly attached to Y.

In some embodiments, R² is an optionally substituted isoxazole. In some embodiments, R² is an optionally substituted isoxazole having a carbon atom of the isoxazole ring directly attached to Y.

In some embodiments, R² is an optionally substituted thiazole. In some embodiments, R² is an optionally substituted thiazole having a carbon atom of the thiazole ring directly attached to Y.

In some embodiments, R² is an optionally substituted isothiazole. In some embodiments, R² is an optionally substituted isothiazole having a carbon atom of the isothiazole ring directly attached to Y.

In some embodiments, R² is an optionally substituted pyrazolidine. In some embodiments, R² is an optionally substituted pyrazolidine having a carbon atom of the pyrazolidine ring directly attached to Y.

In some embodiments, R² is an optionally substituted imidazolidine. In some embodiments, R² is an optionally substituted imidazolidine having a carbon atom of the imidazolidine ring directly attached to Y.

In some embodiments, R² is an optionally substituted pyrazole. In some embodiments, R² is an optionally substituted pyrazole having a carbon atom of the pyrazole ring directly attached to Y.

In some embodiments, R² is an optionally substituted imidazole. In some embodiments, R² is an optionally substituted imidazole having a carbon atom of the imidazole ring directly attached to Y.

In some embodiments, R² is an optionally substituted tetrazole. In some embodiments, R² is an optionally substituted tetrazole having a carbon atom of the tetrazole ring directly attached to Y.

In some embodiments, R² is an optionally substituted sulfolane. In some embodiments, R² is an optionally substituted sulfolane having a carbon atom of the sulfolane ring directly attached to Y.

In some more particular but non-limiting forms, R² is H, —CH₃, —CH₂CH₃, —CH(CH₃)₂, or —C(CH₃)₃. In some embodiments, R² is —CH₂CH₃. In some embodiments, R² is —CH(CH₃)₂.

With respect to any relevant structural representation, such as Formula 3, in some embodiments, R² is CH₃ or C₃₋₁₂ alkyl, such as branched C₃ alkyl (e.g., —CH(CH₃)₂), or linear C₁₋₃ alkyl (e.g., —CH₃, —C₂H₅, —C₃H₇), branched, linear, or cyclic C₃₋₆ alkyl (e.g. —C₃H₇, —C₄H₉, —C₅H₁₁, —C₆H₁₃, —CH(CH₃)₂, —CH(CH₃)(CH₂CH₃), —C(CH₃)₃, —CH(CH₂CH₃)₂, —CH(CH₃)(CH₂CH₂CH₃), —C(CH₃)₂(CH₂CH₃), —CH₂CH₂CH₂CH(CH₃)₂, —CH₂CH(CH₃)CH₂CH₂CH₃, —CH₂CH₂CH(CH₃)CH₂CH₃, —CH(CH₂CH₃)(CH₂CH₂CH₃), —C(CH₃)(CH₂CH₃)₂, cyclopropyl, cyclobutyl, cyclopentyl, cyclohexyl, etc.).

With respect to any relevant structural representation, such as Formula 1, 1C, or 1T, R³ is H, F, Cl, Br, I, OH, OR^{A}, SH, SR^{A}, NH₂, NHR^{A}, NR^{A}R^{B}, CF₃, CN, carboxylic acid, optionally substituted carboxylic ester, or optionally substituted C₁₋₆ alkyl, such as optionally substituted branched C₂₋₆ alkyl or optionally substituted linear C₁₋₆ alkyl, including optionally substituted branched or linear C₁₋₃ alkyl (e.g. —CH₃, —C₂H₅, —C₃H₇), optionally substituted branched, linear, or cyclic C₃₋₆ alkyl (e.g. —C₃H₇, —C₄H₉, —C₅H₁₁, —C₆H₁₃, cyclopropyl, cyclobutyl, cyclopentyl, cyclohexyl, etc.).

In some embodiments, R³ is H. In some embodiments, R³ is F. In some embodiments, R³ is C₁. In some embodiments, R³ is Br. In some embodiments, R³ is I. In some embodiments, R³ is OH. In some embodiments, R³ is OR^{A}. In some embodiments, R³ is SH. In some embodiments, R³ is SR^{A}. In some embodiments, R³ is NH₂. In some embodiments, R³ is NHR^{A}. In some embodiments, R³ is NR^{A}R^{B}. In some embodiments, R³ is CF₃. In some embodiments, R³ is CN. In some embodiments, R³ is CO₂H. In some embodiments, R³ is CO₂R². In some embodiments, R³ is C₁₋₆ alkyl. In some embodiments, R³ is branched C₂₋₆ alkyl. In some embodiments, R³ is —CH₃, —C₂H₅, —C₃H₇, —C₄H₉, —C₅H₁₁, or —C₆H₁₃. In some embodiments, R³ is an optionally substituted C₁₋₆ alkyl. In some embodiments, R³ is an optionally heteroatom substituted linear C₁₋₆ alkyl, such as a linear C₁₋₆ alkyl having polar substituents, including oxygen containing groups (e.g. —OH, =O, OCH₃, etc.), sulfur containing groups (e.g. —SH, —SCH₃, SO₂, SO₃⁻, etc.), nitrogen containing groups (e.g. —NH₂, —NHCH₃, —N(CH₃)₂, —NO₂, —CN, etc.), fluorine containing groups (F, CF₃, CF₂CF₃, CHF₂, CH₂F, CF₂CF₂CF₃, etc.), etc. In some embodiments, R³ is an optionally substituted branched C₃₋₆ alkyl. In some embodiments, R³ is an optionally heteroatom substituted branched C₃₋₆ alkyl, such as a branched C₃₋₆ alkyl having polar substituents, including oxygen containing groups (e.g. —OH, ═O, OCH₃, etc.), sulfur containing groups (e.g. —SH, —SCH₃, SO₂, SO₃⁻, etc.), nitrogen containing groups (e.g. —NH₂, —NHCH₃, —N(CH₃)₂, —NO₂, —CN, etc.), fluorine containing groups (F, CF₃, CF₂CF₃, CHF₂, CH₂F, CF₂CF₂CF₃, etc.), etc. In some embodiments, R³ is H.

With respect to any relevant structural representation, such as Formula 1, 1C, or 1T, each R⁴ is independently H, F, Cl, Br, I, OH, O⁻ (when R⁴ is attached to the nitrogen atom at position 7), OR^{A}, SH, SR^{A}, NH₂, NHR^{A}, NR^{A}R^{B}, CF₃, CN, carboxylic acid (CO₂H), optionally substituted carboxylic ester (such as optionally substituted C₁₋₆ alkyl carboxylic ester, such as optionally substituted branched C₂₋₆ alkyl or linear C₁₋₆ alkyl carboxylic ester, optionally substituted branched C₃ alkyl carboxylic ester (e.g., —CO₂—C(CH₃)₂), or linear C₁₋₃ alkyl carboxylic ester (e.g., —CO₂—CH₃, —CO₂—C₂H₅, —CO₂—C₃H₇), optionally substituted branched, linear, or cyclic C₃₋₆ alkyl carboxylic ester (e.g. —CO₂—C₃H₇, —CO₂—C₄H₉, —CO₂—C₅H₁₁, —CO₂—C₆H₁₃, —CO₂—CH(CH₃)₂, —CO₂—CH(CH₃)(CH₂CH₃), —CO₂—CH(CH₂CH₃)₂, —CO₂—CH(CH₃)(CH₂CH₂CH₃), —CO₂—C(CH₃)₂(CH₂CH₂CH₃), —CO₂—C(CH₃)(CH₂CH₂CH₂CH₃), —CO₂—CH(CH₂CH₃)(CH₂CH₂CH₃), —CO₂-cyclopropyl, —CO₂-cyclobutyl, —CO₂-cyclopentyl, —CO₂-cyclohexyl, etc.), or optionally substituted C₁₋₆ alkyl, such as optionally substituted branched C₂₋₆ alkyl or linear C₁₋₆ alkyl, optionally substituted branched C₃ alkyl (e.g., —C(CH₃)₂), or linear C₁₋₃ alkyl (e.g., —CH₃, —C₂H₅, —C₃H₇), optionally substituted branched, linear, or cyclic C₃₋₆ alkyl (e.g. —C₃H₇, —C₄H₉, —C₅H₁₁, —C₆H₁₃, —CH(CH₃)₂, —CH(CH₃)(CH₂CH₃), —CH(CH₂CH₃)₂, —CH(CH₃)(CH₂CH₂CH₃), —C(CH₃)₂(CH₂CH₂CH₃), —C(CH₃)(CH₂CH₂CH₂CH₃), —CH(CH₂CH₃)(CH₂CH₂CH₃), cyclopropyl, cyclobutyl, cyclopentyl, cyclohexyl, etc.).

In some embodiments, an R⁴ is H. In some embodiments, an R⁴ is F. In some embodiments, an R⁴ is C₁. In some embodiments, an R⁴ is Br. In some embodiments, an R⁴ is I. In some embodiments, an R⁴ is OH. In some embodiments, an R⁴ is OR^{A}. In some embodiments, an R⁴ is SH. In some embodiments, an R⁴ is SR^{A}. In some embodiments, an R⁴ is Nib. In some embodiments, an R⁴ is NHR^{A}. In some embodiments, an R⁴ is NR^{A}R^{B}. In some embodiments, an R⁴ is CF₃. In some embodiments, an R⁴ is CN. In some embodiments, an R⁴ is CO₂H. In some embodiments, an R⁴ is CO₂R^{A}. In some embodiments, an R⁴ is C₁₋₆ alkyl. In some embodiments, an R⁴ is branched C₂₋₆ alkyl. In some embodiments, an R⁴ is —CH₃, —C₂H₅, —C₃H₇, —C₄H₉, —C₅H₁₁, or —C₆H₁₃. In some embodiments, an R⁴ is an optionally substituted C₁₋₆ alkyl. In some embodiments, an R⁴ is an optionally heteroatom substituted linear C₁₋₆ alkyl, such as a linear C₁₋₆ alkyl having polar substituents, including oxygen containing groups (e.g. —OH, O, OCH₃, etc.), sulfur containing groups (e.g. —SH, —SCH₃, SO₂, SO₃⁻, etc.), nitrogen containing groups (e.g. —NH₂, —NHCH₃, —N(CH₃)₂, —NO₂, —CN, etc.), fluorine containing groups (F, CF₃, CF₂CF₃, CHF₂, CH₂F, CF₂CF₂CF₃, etc.). In some embodiments, an R⁴ is an optionally substituted branched C₂₋₆ alkyl. In some embodiments, an R⁴ is an optionally heteroatom substituted branched C₂₋₆ alkyl, such as a branched C₂₋₆ alkyl having polar substituents, including oxygen containing groups (e.g. —OH, =O, OCH₃, etc.), sulfur containing groups (e.g. —SH, —SCH₃, SO₂, SO₃⁻, etc.), nitrogen containing groups (e.g. —NH₂, —NHCH₃, —N(CH₃)₂, —NO₂, —CN, etc.), fluorine containing groups (F, CF₃, CF₂CF₃, CHF₂, CH₂F, CF₂CF₂CF₃, etc.).

In some embodiments, an R⁴ is H.

With respect to any relevant structural representation, such as Formula 1, 1C, or 1T, n is 0, 1, or 2. In some embodiments, n is 0. In some embodiments, n is 1. In some embodiments, n is 2.

With respect to any relevant structural representation, such as Formula 1, 1C, 1T, 2, or 3, X is —, an oxygen atom (O), a sulfur atom (S), or a substituted nitrogen atom (NR⁵ or N⁺R⁵R⁸). In some embodiments, X is —. In some embodiments, X is O. In some embodiments, X is NR⁵. In some embodiments, X is N⁴R⁵R⁸.

In some embodiments, R⁵ is H; optionally substituted C₁₋₆ alkyl, such as optionally substituted branched C₂₋₆ alkyl or linear C₁₋₆ alkyl, optionally substituted branched C₃ alkyl (e.g., —C(CH₃)₂), or linear C₁₋₃ alkyl (e.g., —CH₃, —C₂H₅, —C₃H₇), optionally substituted branched, linear, or cyclic C₃₋₆ alkyl (e.g. —C₃H₇, —C₄H₉, —C₅H₁₁, —C₆H₁₃, —CH(CH₃)₂, —CH(CH₃)(CH₂CH₃), —C(CH₃)₃, —CH(CH₂CH₃)₂, —CH(CH₃)(CH₂CH₂CH₃), —C(CH₃)₂(CH₂CH₃), —CH₂CH₂CH₂CH(CH₃)₂, —CH₂CH(CH₃)CH₂CH₂CH₃, —CH₂CH₂CH(CH₃)CH₂CH₃, —CH(CH₂CH₃)(CH₂CH₂CH₃), —C(CH₃)(CH₂CH₃)₂, cyclopropyl, cyclobutyl, cyclopentyl, cyclohexyl, etc.); optionally substituted carbocycle, including optionally substituted C₃₋₆ cycloalkyl, optionally substituted C₃₋₆ cycloalkenyl, optionally substituted C₃₋₆ cycloalkynyl, optionally substituted phenyl; or optionally substituted heterocycle such as an optionally substituted monocyclic heterocycle having 3 ring carbon atoms and 1 ring oxygen atom, an optionally substituted monocyclic heterocycle having 4 ring carbon atoms and 1 ring oxygen atom, an optionally substituted monocyclic heterocycle having 5 ring carbon atoms and 1 ring oxygen atom, an optionally substituted monocyclic heterocycle having 6 ring carbon atoms and 1 ring oxygen atom, an optionally substituted monocyclic heterocycle having 7 ring carbon atoms and 1 ring oxygen atom, an optionally substituted monocyclic heterocycle having 3 ring carbon atoms and 1 ring sulfur atom, an optionally substituted monocyclic heterocycle having 4 ring carbon atoms and 1 ring sulfur atom, an optionally substituted monocyclic heterocycle having 5 ring carbon atoms and 1 ring sulfur atom, an optionally substituted monocyclic heterocycle having 6 ring carbon atoms and 1 ring sulfur atom, an optionally substituted monocyclic heterocycle having 7 ring carbon atoms and 1 ring sulfur atom, an optionally substituted monocyclic heterocycle having 3 ring carbon atoms and 1 ring nitrogen atom, an optionally substituted monocyclic heterocycle having 4 ring carbon atoms and 1 ring nitrogen atom, an optionally substituted monocyclic heterocycle having 5 ring carbon atoms and 1 ring nitrogen atom, an optionally substituted monocyclic heterocycle having 6 ring carbon atoms and 1 ring nitrogen atom, an optionally substituted monocyclic heterocycle having 7 ring carbon atoms and 1 ring nitrogen atom, an optionally substituted monocyclic heterocycle having 3 ring carbon atoms and 2 ring heteroatoms (N, O, and/or S), an optionally substituted monocyclic heterocycle having 4 ring carbon atoms and 2 ring heteroatoms (N, O, and/or S), an optionally substituted monocyclic heterocycle having 5 ring carbon atoms and 2 ring heteroatoms (N, O, and/or S), an optionally substituted monocyclic heterocycle having 6 ring carbon atoms and 2 ring heteroatoms (N, O, and/or S), an optionally substituted monocyclic heterocycle having 2 ring carbon atoms and 3 ring heteroatoms (N, O, and/or S), an optionally substituted monocyclic heterocycle having 3 ring carbon atoms and 3 ring heteroatoms (N, O, and/or S), an optionally substituted monocyclic heterocycle having 4 ring carbon atoms and 3 ring heteroatoms (N, O, and/or S), an optionally substituted monocyclic heterocycle having 5 ring carbon atoms and 3 ring heteroatoms (N, O, and/or S), an optionally substituted bicyclic heterocycle having 6 ring carbon atoms and 1 ring heteroatom (N, O, or S), an optionally substituted bicyclic heterocycle having 7 ring carbon atoms and 1 ring heteroatom (N, O, or S), an optionally substituted bicyclic heterocycle having 8 ring carbon atoms and 1 ring heteroatom (N, O, or S), an optionally substituted bicyclic heterocycle having 9 ring carbon atoms and 1 ring heteroatom (N, O, or S), an optionally substituted bicyclic heterocycle having 10 ring carbon atoms and 1 ring heteroatom (N, O, or S), an optionally substituted bicyclic heterocycle having 11 ring carbon atoms and 1 ring heteroatom (N, O, or S), an optionally substituted bicyclic heterocycle having 12 ring carbon atoms and 1 ring heteroatom (N, O, or S), an optionally substituted bicyclic heterocycle having 5 ring carbon atoms and 2 ring heteroatoms (N, O, and/or S), an optionally substituted bicyclic heterocycle having 6 ring carbon atoms and 2 ring heteroatoms (N, O, and/or S), an optionally substituted bicyclic heterocycle having 7 ring carbon atoms and 2 ring heteroatoms (N, O, and/or S), an optionally substituted bicyclic heterocycle having 8 ring carbon atoms and 2 ring heteroatoms (N, O, and/or S), an optionally substituted bicyclic heterocycle having 9 ring carbon atoms and 2 ring heteroatoms (N, O, and/or S), an optionally substituted bicyclic heterocycle having 10 ring carbon atoms and 2 ring heteroatoms (N, O, and/or S), an optionally substituted bicyclic heterocycle having 11 ring carbon atoms and 2 ring heteroatoms (N, O, and/or S), an optionally substituted bicyclic heterocycle having 5 ring carbon atoms and 3 ring heteroatoms (N, O, and/or S), an optionally substituted bicyclic heterocycle having 6 ring carbon atoms and 3 ring heteroatoms (N, O, and/or S), an optionally substituted bicyclic heterocycle having 7 ring carbon atoms and 3 ring heteroatoms (N, O, and/or S), an optionally substituted bicyclic heterocycle having 8 ring carbon atoms and 3 ring heteroatoms (N, O, and/or S), an optionally substituted bicyclic heterocycle having 9 ring carbon atoms and 3 ring heteroatoms (N, O, and/or S), an optionally substituted bicyclic heterocycle having 10 ring carbon atoms and 3 ring heteroatoms (N, O, and/or S), optionally substituted oxetane, optionally substituted tetrahydrofuran, optionally substituted dihydrofuran, optionally substituted furan, optionally substituted furanone, optionally substituted tetrahydropyran, optionally substituted dihydropyran, an optionally substituted pyran, optionally substituted tetrahydropyrone, optionally substituted dihydropyrone, optionally substituted pyrone, optionally substituted thietane, optionally substituted tetrahydrothiophene, optionally substituted dihydrothiophene, an optionally substituted thiophene, optionally substituted azetidine, optionally substituted pyrrolidine, optionally substituted pyrroline, optionally substituted pyrrole, optionally substituted piperidine, optionally substituted pyridine, optionally substituted oxazole, optionally substituted isoxazole, optionally substituted thiazole, optionally substituted isothiazole, optionally substituted pyrazolidine, optionally substituted imidazolidine, optionally substituted pyrazole, optionally substituted imidazole, optionally substituted tetrazole, optionally substituted sulfolane.

In some embodiments, R⁵ is H. In some embodiments, R⁵ is CH₃.

In some embodiments, when X is a substituted nitrogen atom, the N, R¹, and R⁵ may together form an optionally substituted heterocyclic ring. In some embodiments, N, R¹, and R⁵ may together form an optionally substituted morpholine ring. In some embodiments, N, R¹, and R⁵ may together form an optionally substituted piperidine ring. In some embodiments, N, R¹, and R⁵ may together form an optionally substituted piperazine ring.

In some embodiments, R⁸ is H; optionally substituted C₁₋₆ alkyl, such as optionally substituted branched C₂₋₆ alkyl or linear C₁₋₆ alkyl, optionally substituted branched C₃ alkyl (e.g., —C(CH₃)₂), or linear C₁₋₃ alkyl (e.g., —CH₃, —C₂H₅, —C₃H₇), optionally substituted branched, linear, or cyclic C₃₋₆ alkyl (e.g. —C₃H₇, —C₄H₉, —C₅H₁₁, —C₆H₁₃, —CH(CH₃)₂, —CH(CH₃)(CH₂CH₃), —C(CH₃)₃, —CH(CH₂CH₃)₂, —CH(CH₃)(CH₂CH₂CH₃), —C(CH₃)₂(CH₂CH₃), —CH₂CH₂CH₂CH(CH₃)₂, —CH₂CH(CH₃)CH₂CH₂CH₃, —CH₂CH₂CH(CH₃)CH₂CH₃, —CH(CH₂CH₃)(CH₂CH₂CH₃), —C(CH₃)(CH₂CH₃)₂, cyclopropyl, cyclobutyl, cyclopentyl, cyclohexyl, etc.); optionally substituted carbocycle, including optionally substituted C₃₋₆ cycloalkyl, optionally substituted C₃₋₆ cycloalkenyl, optionally substituted C₃₋₆ cycloalkynyl, optionally substituted phenyl; or optionally substituted heterocycle such as an optionally substituted monocyclic heterocycle having 3 ring carbon atoms and 1 ring oxygen atom, an optionally substituted monocyclic heterocycle having 4 ring carbon atoms and 1 ring oxygen atom, an optionally substituted monocyclic heterocycle having 5 ring carbon atoms and 1 ring oxygen atom, an optionally substituted monocyclic heterocycle having 6 ring carbon atoms and 1 ring oxygen atom, an optionally substituted monocyclic heterocycle having 7 ring carbon atoms and 1 ring oxygen atom, an optionally substituted monocyclic heterocycle having 3 ring carbon atoms and 1 ring sulfur atom, an optionally substituted monocyclic heterocycle having 4 ring carbon atoms and 1 ring sulfur atom, an optionally substituted monocyclic heterocycle having 5 ring carbon atoms and 1 ring sulfur atom, an optionally substituted monocyclic heterocycle having 6 ring carbon atoms and 1 ring sulfur atom, an optionally substituted monocyclic heterocycle having 7 ring carbon atoms and 1 ring sulfur atom, an optionally substituted monocyclic heterocycle having 3 ring carbon atoms and 1 ring nitrogen atom, an optionally substituted monocyclic heterocycle having 4 ring carbon atoms and 1 ring nitrogen atom, an optionally substituted monocyclic heterocycle having 5 ring carbon atoms and 1 ring nitrogen atom, an optionally substituted monocyclic heterocycle having 6 ring carbon atoms and 1 ring nitrogen atom, an optionally substituted monocyclic heterocycle having 7 ring carbon atoms and 1 ring nitrogen atom, an optionally substituted monocyclic heterocycle having 3 ring carbon atoms and 2 ring heteroatoms (N, O, and/or S), an optionally substituted monocyclic heterocycle having 4 ring carbon atoms and 2 ring heteroatoms (N, O, and/or S), an optionally substituted monocyclic heterocycle having 5 ring carbon atoms and 2 ring heteroatoms (N, O, and/or S), an optionally substituted monocyclic heterocycle having 6 ring carbon atoms and 2 ring heteroatoms (N, O, and/or S), an optionally substituted monocyclic heterocycle having 2 ring carbon atoms and 3 ring heteroatoms (N, O, and/or S), an optionally substituted monocyclic heterocycle having 3 ring carbon atoms and 3 ring heteroatoms (N, O, and/or S), an optionally substituted monocyclic heterocycle having 4 ring carbon atoms and 3 ring heteroatoms (N, O, and/or S), an optionally substituted monocyclic heterocycle having 5 ring carbon atoms and 3 ring heteroatoms (N, O, and/or S), an optionally substituted bicyclic heterocycle having 6 ring carbon atoms and 1 ring heteroatom (N, O, or S), an optionally substituted bicyclic heterocycle having 7 ring carbon atoms and 1 ring heteroatom (N, O, or S), an optionally substituted bicyclic heterocycle having 8 ring carbon atoms and 1 ring heteroatom (N, O, or S), an optionally substituted bicyclic heterocycle having 9 ring carbon atoms and 1 ring heteroatom (N, O, or S), an optionally substituted bicyclic heterocycle having 10 ring carbon atoms and 1 ring heteroatom (N, O, or S), an optionally substituted bicyclic heterocycle having 11 ring carbon atoms and 1 ring heteroatom (N, O, or S), an optionally substituted bicyclic heterocycle having 12 ring carbon atoms and 1 ring heteroatom (N, O, or S), an optionally substituted bicyclic heterocycle having 5 ring carbon atoms and 2 ring heteroatoms (N, O, and/or S), an optionally substituted bicyclic heterocycle having 6 ring carbon atoms and 2 ring heteroatoms (N, O, and/or S), an optionally substituted bicyclic heterocycle having 7 ring carbon atoms and 2 ring heteroatoms (N, O, and/or S), an optionally substituted bicyclic heterocycle having 8 ring carbon atoms and 2 ring heteroatoms (N, O, and/or S), an optionally substituted bicyclic heterocycle having 9 ring carbon atoms and 2 ring heteroatoms (N, O, and/or S), an optionally substituted bicyclic heterocycle having 10 ring carbon atoms and 2 ring heteroatoms (N, O, and/or S), an optionally substituted bicyclic heterocycle having 11 ring carbon atoms and 2 ring heteroatoms (N, O, and/or S), an optionally substituted bicyclic heterocycle having 5 ring carbon atoms and 3 ring heteroatoms (N, O, and/or S), an optionally substituted bicyclic heterocycle having 6 ring carbon atoms and 3 ring heteroatoms (N, O, and/or S), an optionally substituted bicyclic heterocycle having 7 ring carbon atoms and 3 ring heteroatoms (N, O, and/or S), an optionally substituted bicyclic heterocycle having 8 ring carbon atoms and 3 ring heteroatoms (N, O, and/or S), an optionally substituted bicyclic heterocycle having 9 ring carbon atoms and 3 ring heteroatoms (N, O, and/or S), an optionally substituted bicyclic heterocycle having 10 ring carbon atoms and 3 ring heteroatoms (N, O, and/or S), optionally substituted oxetane, optionally substituted tetrahydrofuran, optionally substituted dihydrofuran, optionally substituted furan, optionally substituted furanone, optionally substituted tetrahydropyran, optionally substituted dihydropyran, an optionally substituted pyran, optionally substituted tetrahydropyrone, optionally substituted dihydropyrone, optionally substituted pyrone, optionally substituted thietane, optionally substituted tetrahydrothiophene, optionally substituted dihydrothiophene, an optionally substituted thiophene, optionally substituted azetidine, optionally substituted pyrrolidine, optionally substituted pyrroline, optionally substituted pyrrole, optionally substituted piperidine, optionally substituted pyridine, optionally substituted oxazole, optionally substituted isoxazole, optionally substituted thiazole, optionally substituted isothiazole, optionally substituted pyrazolidine, optionally substituted imidazolidine, optionally substituted pyrazole, optionally substituted imidazole, optionally substituted tetrazole, optionally substituted sulfolane.

In some embodiments, R⁸ is H. In some embodiments, R⁸ is CH₃.

In some embodiments, when X is a substituted nitrogen atom, the N, R⁵, and R⁸ may together form an optionally substituted heterocyclic ring. In some embodiments, N, R⁵, and R⁸ may together form an optionally substituted morpholine ring. In some embodiments, N, R⁵, and R⁸ may together form an optionally substituted piperidine ring. In some embodiments, N, R⁵, and R⁸ may together form an optionally substituted piperazine ring.

With respect to any relevant structural representation, such as Formula 1, 1C, 1T, or 2, Y is —, —O—,

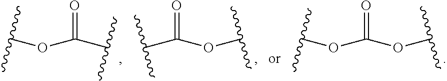

In some embodiments, Y is —. In some embodiments, Y is —O—. In some embodiments, Y is

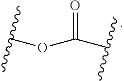 [SMILES: *OC(*)=O]

In some embodiments, Y is

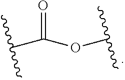 [SMILES: *OC(*)=O]

In some embodiments, Y is

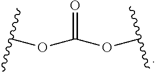 [SMILES: *OC(=O)O*]

In some embodiments, Y is — or

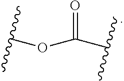 [SMILES: *OC(*)=O]

In some embodiments, R¹ is alkyl, Y is O, and R² is alkyl.

In some embodiments, R¹ alkyl, Y is

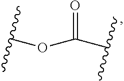 [SMILES: *OC(*)=O]

and R is alkyl.

In some embodiments, R¹ is —, Y is —, and R² is cycloalkyl.

In some embodiments, R¹ is —, Y is —, and R² is aryl.

In some embodiments, R¹ is —, Y is —, and R² is heteroaryl.

In some embodiments, R¹ is alkyl, Y is —, and R² is aryl.

In some embodiments, R¹ is —, Y is —, and R² is cycloalkyl.

In some embodiments, R¹ is —, Y is —, and R² is a heterocycle.

In some embodiments, R¹ is alkyl, Y is

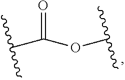 [SMILES: *OC(*)=O]

and R² is alkyl.

With respect to any relevant structural representation, such as Formula 1, 1C, or 1T, Z is —; optionally substituted hydrocarbyl, such as an optionally substituted C₁₋₁₂ hydrocarbon group, including optionally substituted alkyl, including optionally substituted C₁₋₁₂ alkyl, such as optionally substituted branched C₂₋₁₂ alkyl or optionally substituted linear C₁₋₁₂ alkyl, including optionally substituted branched C₂₋₆ alkyl or linear C₁₋₆ alkyl, optionally substituted branched C₂₋₃ alkyl (e.g., —CH(CH₃)—, —CH(CH₂CH₃)—, —C(CH₃)₂—), or linear C₁₋₃ alkyl (e.g., —CH₂—, —C₂H₄—, —C₃H₆—), optionally substituted branched, linear, or cyclic C₃₋₆ alkyl (e.g. —C₃H₆—, —C₄H₅—, —C₅H₁₀—, —C₆H₁₂—, —CH(CH₂CH₃), —C(CH₃)₂—, —C(CH₃)(CH₂CH₃), —CH(CH₂CH₂CH₃), —C(CH₂CH₃)₂—, —C(CH₃)(CH₂CH₂CH₃), —CH(CH₂CH₂CH₂CH₃)—, —C(CH₃)(CH₂CH₂CH₂CH₃)—, —CH(CH₂CH₂CH₂CH₂CH₃), —C(CH₂CH₃)(CH₂CH₂CH₃)—, cyclopropyl, cyclobutyl, cyclopentyl, cyclohexyl, etc.), optionally substituted branched, linear, or cyclic C₆₋₉ alkyl (e.g., —C(CH₃)(CH₂CH₂CH₂CH₃)—, —CH(CH₂CH₂CH₂CH₂CH₃)—, —C(CH₂CH₃)(CH₂CH₂CH₃), —C₆H₁₂—, —C₇H₁₄—, —C₈H₁₆—, —C₉H₁₈—, cyclohexyl, cycloheptyl, cyclooctyl, cyclononyl, etc.), optionally substituted branched, linear, or cyclic C₉₋₁₂ alkyl, C₂₋₁₂ alkenyl, C₂₋₁₂ alkynyl, optionally substituted benzyl, etc.; optionally substituted carbocycle, including optionally substituted C₃₋₁₂ cycloalkyl, optionally substituted C₃₋₆ cycloalkyl, optionally substituted C₆₋₉ cycloalkyl, optionally substituted C₉₋₁₂ cycloalkyl, optionally substituted C₃₋₁₂ cycloalkenyl, optionally substituted C₃₋₆ cycloalkenyl, optionally substituted C₆₋₉ cycloalkenyl, optionally substituted C₉₋₁₂ cycloalkenyl, optionally substituted C₃₋₁₂ cycloalkynyl, optionally substituted C₃₋₆ cycloalkynyl, optionally substituted C₆₋₉ cycloalkynyl, optionally substituted C₉₋₁₂ cycloalkynyl, optionally substituted phenyl, optionally substituted naphthyl, or optionally substituted heterocycle such as an optionally substituted monocyclic heterocycle having 3 ring carbon atoms and 1 ring oxygen atom, an optionally substituted monocyclic heterocycle having 4 ring carbon atoms and 1 ring oxygen atom, an optionally substituted monocyclic heterocycle having 5 ring carbon atoms and 1 ring oxygen atom, an optionally substituted monocyclic heterocycle having 6 ring carbon atoms and 1 ring oxygen atom, an optionally substituted monocyclic heterocycle having 7 ring carbon atoms and 1 ring oxygen atom, an optionally substituted monocyclic heterocycle having 3 ring carbon atoms and 1 ring sulfur atom, an optionally substituted monocyclic heterocycle having 4 ring carbon atoms and 1 ring sulfur atom, an optionally substituted monocyclic heterocycle having 5 ring carbon atoms and 1 ring sulfur atom, an optionally substituted monocyclic heterocycle having 6 ring carbon atoms and 1 ring sulfur atom, an optionally substituted monocyclic heterocycle having 7 ring carbon atoms and 1 ring sulfur atom, an optionally substituted monocyclic heterocycle having 3 ring carbon atoms and 1 ring nitrogen atom, an optionally substituted monocyclic heterocycle having 4 ring carbon atoms and 1 ring nitrogen atom, an optionally substituted monocyclic heterocycle having 5 ring carbon atoms and 1 ring nitrogen atom, an optionally substituted monocyclic heterocycle having 6 ring carbon atoms and 1 ring nitrogen atom, an optionally substituted monocyclic heterocycle having 7 ring carbon atoms and 1 ring nitrogen atom, an optionally substituted monocyclic heterocycle having 3 ring carbon atoms and 2 ring heteroatoms (N, O, and/or S), an optionally substituted monocyclic heterocycle having 4 ring carbon atoms and 2 ring heteroatoms (N, O, and/or S), an optionally substituted monocyclic heterocycle having 5 ring carbon atoms and 2 ring heteroatoms (N, O, and/or S), an optionally substituted monocyclic heterocycle having 6 ring carbon atoms and 2 ring heteroatoms (N, O, and/or S), an optionally substituted monocyclic heterocycle having 2 ring carbon atoms and 3 ring heteroatoms (N, O, and/or S), an optionally substituted monocyclic heterocycle having 3 ring carbon atoms and 3 ring heteroatoms (N, O, and/or S), an optionally substituted monocyclic heterocycle having 4 ring carbon atoms and 3 ring heteroatoms (N, O, and/or S), an optionally substituted monocyclic heterocycle having 5 ring carbon atoms and 3 ring heteroatoms (N, O, and/or S), an optionally substituted bicyclic heterocycle having 6 ring carbon atoms and 1 ring heteroatom (N, O, or S), an optionally substituted bicyclic heterocycle having 7 ring carbon atoms and 1 ring heteroatom (N, O, or S), an optionally substituted bicyclic heterocycle having 8 ring carbon atoms and 1 ring heteroatom (N, O, or S), an optionally substituted bicyclic heterocycle having 9 ring carbon atoms and 1 ring heteroatom (N, O, or S), an optionally substituted bicyclic heterocycle having 10 ring carbon atoms and 1 ring heteroatom (N, O, or S), an optionally substituted bicyclic heterocycle having 11 ring carbon atoms and 1 ring heteroatom (N, O, or S), an optionally substituted bicyclic heterocycle having 12 ring carbon atoms and 1 ring heteroatom (N, O, or S), an optionally substituted bicyclic heterocycle having 5 ring carbon atoms and 2 ring heteroatoms (N, O, and/or S), an optionally substituted bicyclic heterocycle having 6 ring carbon atoms and 2 ring heteroatoms (N, O, and/or S), an optionally substituted bicyclic heterocycle having 7 ring carbon atoms and 2 ring heteroatoms (N, O, and/or S), an optionally substituted bicyclic heterocycle having 8 ring carbon atoms and 2 ring heteroatoms (N, O, and/or S), an optionally substituted bicyclic heterocycle having 9 ring carbon atoms and 2 ring heteroatoms (N, O, and/or S), an optionally substituted bicyclic heterocycle having 10 ring carbon atoms and 2 ring heteroatoms (N, O, and/or S), an optionally substituted bicyclic heterocycle having 11 ring carbon atoms and 2 ring heteroatoms (N, O, and/or S), an optionally substituted bicyclic heterocycle having 5 ring carbon atoms and 3 ring heteroatoms (N, O, and/or S), an optionally substituted bicyclic heterocycle having 6 ring carbon atoms and 3 ring heteroatoms (N, O, and/or S), an optionally substituted bicyclic heterocycle having 7 ring carbon atoms and 3 ring heteroatoms (N, O, and/or S), an optionally substituted bicyclic heterocycle having 8 ring carbon atoms and 3 ring heteroatoms (N, O, and/or S), an optionally substituted bicyclic heterocycle having 9 ring carbon atoms and 3 ring heteroatoms (N, O, and/or S), an optionally substituted bicyclic heterocycle having 10 ring carbon atoms and 3 ring heteroatoms (N, O, and/or S), optionally substituted oxetane, optionally substituted tetrahydrofuran, optionally substituted dihydrofuran, optionally substituted furan, optionally substituted furanone, optionally substituted tetrahydropyran, optionally substituted dihydropyran, an optionally substituted pyran, optionally substituted tetrahydropyrone, optionally substituted dihydropyrone, optionally substituted pyrone, optionally substituted thietane, optionally substituted tetrahydrothiophene, optionally substituted dihydrothiophene, an optionally substituted thiophene, optionally substituted azetidine, optionally substituted pyrrolidine, optionally substituted pyrroline, optionally substituted pyrrole, optionally substituted piperidine, optionally substituted pyridine, optionally substituted oxazole, optionally substituted isoxazole, optionally substituted thiazole, optionally substituted isothiazole, optionally substituted pyrazolidine, optionally substituted imidazolidine, optionally substituted pyrazole, optionally substituted imidazole, optionally substituted tetrazole, optionally substituted sulfolane.

Potential substituents on Z include alkyl, such as C₁₋₁₂ alkyl, C₁₋₃ alkyl, C₃₋₆ alkyl, C₆₋₉ alkyl, C₉₋₁₂ alkyl, CH₃, —C₂H₅, —C₃H₇, —C₄H₉, —C₅H₁₁, —C₆H₁₃, —C₇H₁₅, —C₈H₁₇, —C₉H₁₉, —C₁₀H₂₁, —C₁₁H₂₃, —C₁₂H₂₅, etc.; halo, such as F, Cl, Br, I, etc.; OH; —CO₂H; acyl, such as C₁₋₁₂—C(═O)-alkyl, C₁₋₃—C(═O)-alkyl, C₃₋₆—C(═O)-alkyl, C₆₋₉—C(═O)-alkyl, C₉₋₁₂—C(═O)-alkyl, —C(═O)—CH₃, —C(═O)—C₂H₅, —C(═O)—C₃H₇, —C(═O)—C₄H₉, —C(═O)—C₅H₁₁, —C(═O)—C₆H₁₃, —C(═O)—C₇H₁₅, —C(═O)—C₈H₁₇, —C(═O)—C₉H₁₉, —C(═O)—C₁₀H₂₁, —C(═O)—C₁₁H₂₃, —C(═O)—C₁₂H₂₅, —C(═O)-phenyl, etc.; alkoxy, such as such as C₁₋₁₂—O-alkyl, C₁₋₃—O-alkyl, C₃₋₆—O-alkyl, C₆₋₉—O-alkyl, C₉₋₁₂—O-alkyl, —OCH₃, —OC₂H₅, —OC₃H₇, —OC₄H₉, —OC₅H₁₁, —OC₆H₁₃, —OC₇H₁₅, —OC₈H₁₇, —OC₉H₁₉, —OC₁₀H₂₁, —OC₁₁H₂₃, —OC₁₂H₂₅, etc.; alkylthio, such as C₁₋₁₂—S-alkyl, C₁₋₃—S-alkyl, C₃₋₆—S-alkyl, C₆₋₉—S-alkyl, C₉₋₁₂—S-alkyl, —SCH₃, —SC₂H₅, —SC₃H₇, —SC₄H₉, —SC₅H₁₁, —SC₆H₁₃, —SC₇H₁₅, —SC₈H₁₇, —SC₉H₁₉, —SC₁₀H₂₁, —SC₁₁H₂₃, —SC₁₂H₂₅, etc.; thioester (e.g. —C(O)SR^{A}, —SC(O)R^{A}, etc.); phosphoryl; amino (e.g. NR^{A}R^{B}, where NR^{A}R^{B} may potentially form a ring, or N⁺R^{A}R^{B}R^{C}, wherein R^{C} is H or hydrocarbyl, and N⁺R^{A}R^{B}R^{C} may potentially form a ring); amide (e.g. —C(═O)NR^{A}R^{B}, including where NR^{A}R^{B} form a ring); CN; —NO₂; azido; alkenyl, such as C₂₋₁₂ alkenyl, C₂₋₄ alkenyl, C₄₋₆ alkenyl, C₆₋₈ alkenyl, C₈₋₁₀ alkenyl, C₁₀₋₁₂ alkenyl, (e.g. —CH═CH₂, —CH═CH₂CH₃, etc.); alkynyl, such as C₂₋₁₂ alkynyl, C₂₋₄ alkynyl, C₄₋₆ alkynyl, C₆₋₈ alkynyl, C₈₋₁₀ alkynyl, C₁₀₋₁₂ alkynyl, (e.g. —C≡CH, —C≡C—CH₃, etc.); cycloalkyl, such as cyclopropyl, cyclobutyl, cyclopentyl, cyclohexyl, cyclohexyl, etc.; heterocyclylalkyl (e.g. -alkyl-heterocycle); heteroaralkyl (e.g. -alkyl-heteroaryl); sulfonamide (e.g. —SO₂NR^{A}R^{B}, —NR^{A}SO₂R^{B}, etc.); aryl; heteroaryl; heterocyclyl; aralkyl (e.g. -alkyl-aryl; etc.

In some embodiments, Z is —. In some embodiments, Z is alkyl, such as C₁₋₁₂ alkyl. In some embodiments, Z is linear C₁₋₁₂ alkyl. In some embodiments, Z is branched C₂₋₁₂ alkyl. In some embodiments, Z is —CH₂⁺³¹, —C₂H₄⁺³¹, —C₃H₆—, —C₃H₆⁺³¹, —C₄H₈—, —C₅H₁₀⁺³¹, —C₆H₁₂—, —C₇H₁₄—, —C₈H₁₆—, or —C₉H₁₈—, In some embodiments, Z is an optionally substituted linear C₁₋₁₂ alkyl. In some embodiments, Z is an optionally substituted branched C₂₋₁₂ alkyl. In some embodiments, Z is —CH₂—.

In some embodiments, Z is an optionally substituted carbocycle. In some embodiments, Z is optionally substituted cyclohexyl. In some embodiments, Z is an optionally substituted aryl. In some embodiments, Z is an optionally substituted phenyl. In some embodiments, Z is an optionally substituted benzyl. In some embodiments, Z is an optionally substituted heteroaryl. In some embodiments, Z is an optionally substituted heterocycle. In some embodiments wherein Z is an optionally substituted heterocycle, a carbon atom of the heterocycle (rather than a heteroatom of the heterocycle) is directly attached to the indole nitrogen atom of the core azaindole ring. In some aspects, Z is an electron-deficient heterocyclic moiety. In some aspects, Z is an electron-deficient aryl moiety. In some aspects, Z is an electron-deficient alkyl moiety.

In some embodiments, Z is an optionally substituted benzyl. In some embodiments, Z is an optionally substituted benzyl having the carbon atom of the methylene directly attached to the indole nitrogen atom of the core azaindole ring.

In some embodiments, Z is an optionally substituted phenyl. In some embodiments, Z is an optionally substituted phenyl having a carbon atom of the phenyl directly attached to the indole nitrogen atom of the core azaindole ring.

In some embodiments, Z is an optionally substituted oxetane. In some embodiments, Z is an optionally substituted oxetane having a carbon atom of the oxetane ring directly attached to the indole nitrogen atom of the core azaindole ring.

In some embodiments, Z is an optionally substituted tetrahydrofuran. In some embodiments, Z is an optionally substituted tetrahydrofuran having a carbon atom of the tetrahydrofuran ring directly attached to the indole nitrogen atom of the core azaindole ring.

In some embodiments, Z is an optionally substituted dihydrofuran. In some embodiments, Z is an optionally substituted dihydrofuran having a carbon atom of the dihydrofuran ring directly attached to the indole nitrogen atom of the core azaindole ring.

In some embodiments, Z is an optionally substituted furan. In some embodiments, Z is an optionally substituted furan having a carbon atom of the furan ring directly attached to the indole nitrogen atom of the core azaindole ring.

In some embodiments, Z is an optionally substituted furanone. In some embodiments, Z is an optionally substituted furanone having a carbon atom of the furanone ring directly attached to the indole nitrogen atom of the core azaindole ring.

In some embodiments, Z is an optionally substituted tetrahydropyran. In some embodiments, Z is an optionally substituted tetrahydropyran having a carbon atom of the tetrahydropyran ring directly attached to the indole nitrogen atom of the core azaindole ring.

In some embodiments, Z is an optionally substituted dihydropyran. In some embodiments, Z is an optionally substituted dihydropyran having a carbon atom of the dihydropyran ring directly attached to the indole nitrogen atom of the core azaindole ring.

In some embodiments, Z is an optionally substituted pyran. In some embodiments, Z is an optionally substituted pyran having a carbon atom of the pyran ring directly attached to the indole nitrogen atom of the core azaindole ring.

In some embodiments, Z is an optionally substituted tetrahydropyrone. In some embodiments, Z is an optionally substituted tetrahydropyrone having a carbon atom of the tetrahydropyrone ring directly attached to the indole nitrogen atom of the core azaindole ring.

In some embodiments, Z is an optionally substituted dihydropyrone. In some embodiments, Z is an optionally substituted dihydropyrone having a carbon atom of the dihydropyrone ring directly attached to the indole nitrogen atom of the core azaindole ring.

In some embodiments, Z is an optionally substituted pyrone. In some embodiments, Z is an optionally substituted pyrone having a carbon atom of the pyrone ring directly attached to the indole nitrogen atom of the core azaindole ring.

In some embodiments, Z is an optionally substituted thietane. In some embodiments, Z is an optionally substituted thietane having a carbon atom of the thietane ring directly attached to the indole nitrogen atom of the core azaindole ring.

In some embodiments, Z is an optionally substituted tetrahydrothiophene. In some embodiments, Z is an optionally substituted tetrahydrothiophene having a carbon atom of the tetrahydrothiophene ring directly attached to the indole nitrogen atom of the core azaindole ring.

In some embodiments, Z is an optionally substituted dihydrothiophene. In some embodiments, Z is an optionally substituted dihydrothiophene having a carbon atom of the dihydrothiophene ring directly attached to the indole nitrogen atom of the core azaindole ring.

In some embodiments, Z is an optionally substituted thiophene. In some embodiments, Z is an optionally substituted thiophene having a carbon atom of the thiophene ring directly attached to the indole nitrogen atom of the core azaindole ring.

In some embodiments, Z is an optionally substituted azetidine. In some embodiments, Z is an optionally substituted azetidine having a carbon atom of the azetidine ring directly attached to the indole nitrogen atom of the core azaindole ring. In some embodiments, Z is azetidine having an optionally substituted diphenylmethyl substituent. In some embodiments, Z is azetidine having an optionally substituted diphenylmethyl substituent attached to the nitrogen atom of the azetidine ring.

In some embodiments, Z is an optionally substituted pyrrolidine. In some embodiments, Z is an optionally substituted pyrrolidine having a carbon atom of the pyrrolidine ring directly attached to the indole nitrogen atom of the core azaindole ring.

In some embodiments, Z is an optionally substituted pyrroline. In some embodiments, Z is an optionally substituted pyrroline having a carbon atom of the pyrroline ring directly attached to the indole nitrogen atom of the core azaindole ring.

In some embodiments, Z is an optionally substituted pyrrole. In some embodiments, Z is an optionally substituted pyrrole having a carbon atom of the pyrrole ring directly attached to the indole nitrogen atom of the core azaindole ring.

In some embodiments, Z is an optionally substituted piperidine. In some embodiments, Z is an optionally substituted piperidine having a carbon atom of the piperidine ring directly attached to the indole nitrogen atom of the core azaindole ring.

In some embodiments, Z is an optionally substituted pyridine. In some embodiments, Z is an optionally substituted pyridine having a carbon atom of the pyridine ring directly attached to the indole nitrogen atom of the core azaindole ring.

In some embodiments, Z is an optionally substituted oxazole. In some embodiments, Z is an optionally substituted oxazole having a carbon atom of the oxazole ring directly attached to the indole nitrogen atom of the core azaindole ring.

In some embodiments, Z is an optionally substituted isoxazole. In some embodiments, Z is an optionally substituted isoxazole having a carbon atom of the isoxazole ring directly attached to the indole nitrogen atom of the core azaindole ring.

In some embodiments, Z is an optionally substituted thiazole. In some embodiments, Z is an optionally substituted thiazole having a carbon atom of the thiazole ring directly attached to the indole nitrogen atom of the core azaindole ring.

In some embodiments, Z is an optionally substituted isothiazole. In some embodiments, Z is an optionally substituted isothiazole having a carbon atom of the isothiazole ring directly attached to the indole nitrogen atom of the core azaindole ring.

In some embodiments, Z is an optionally substituted pyrazolidine. In some embodiments, Z is an optionally substituted pyrazolidine having a carbon atom of the pyrazolidine ring directly attached to the indole nitrogen atom of the core azaindole ring.

In some embodiments, Z is an optionally substituted imidazolidine. In some embodiments, Z is an optionally substituted imidazolidine having a carbon atom of the imidazolidine ring directly attached to the indole nitrogen atom of the core azaindole ring.

In some embodiments, Z is an optionally substituted pyrazole. In some embodiments, Z is an optionally substituted pyrazole having a carbon atom of the pyrazole ring directly attached to the indole nitrogen atom of the core azaindole ring.

In some embodiments, Z is an optionally substituted imidazole. In some embodiments, Z is an optionally substituted imidazole having a carbon atom of the imidazole ring directly attached to the indole nitrogen atom of the core azaindole ring.

In some embodiments, Z is an optionally substituted tetrazole. In some embodiments, Z is an optionally substituted tetrazole having a carbon atom of the tetrazole ring directly attached to the indole nitrogen atom of the core azaindole ring.

In some embodiments, Z is an optionally substituted sulfolane. In some embodiments, Z is an optionally substituted sulfolane having a carbon atom of the sulfolane ring directly attached to the indole nitrogen atom of the core azaindole ring.

In some embodiments, Z is represented by one of the following structures:

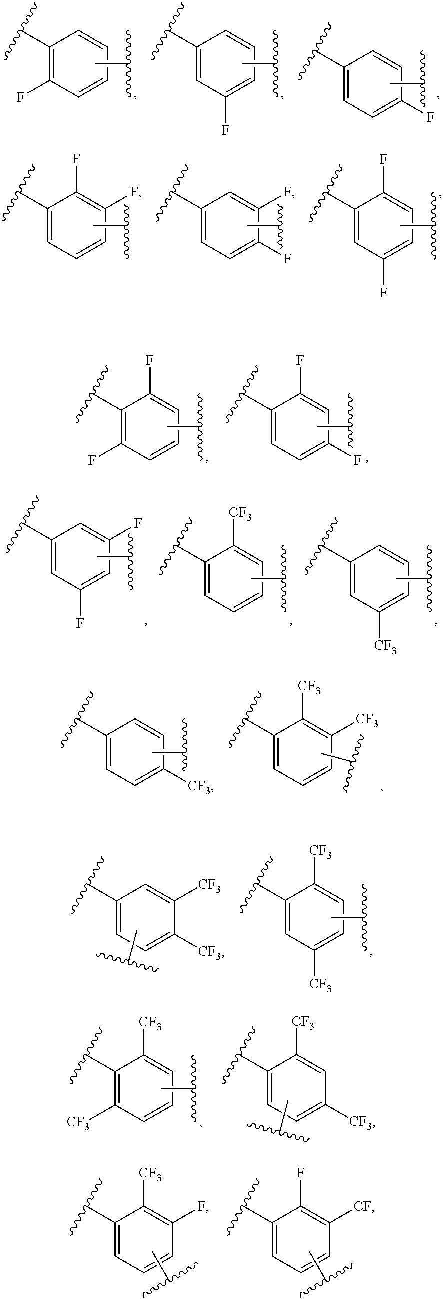

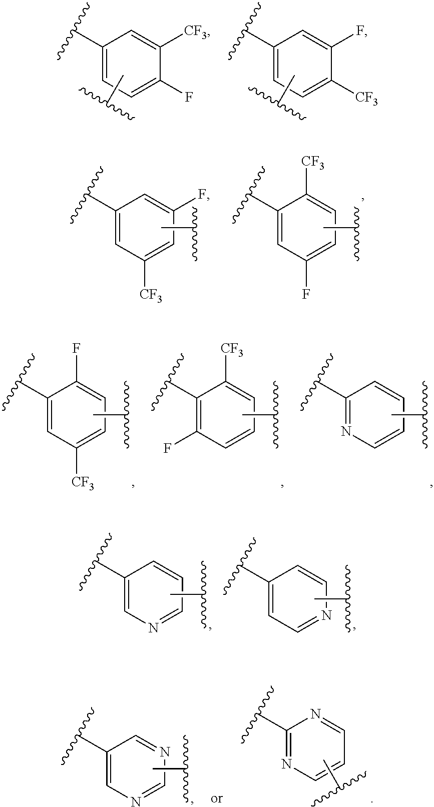

In some embodiments, Z is

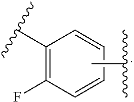

In some embodiments, Z is

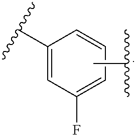

In some embodiments, Z is

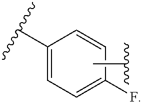

In some embodiments, Z is

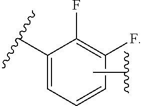

In some embodiments, Z is

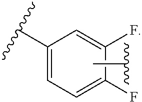

In some embodiments, Z is

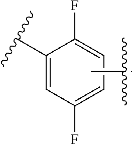

In some embodiments, Z is

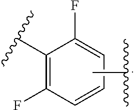

In some embodiments, Z is

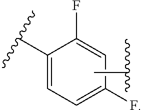

In some embodiments, Z is

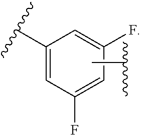

In some embodiments, Z is

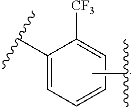

In some embodiments, Z is

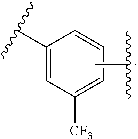

In some embodiments, Z is

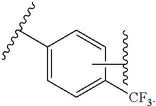

In some embodiments, Z is. In some embodiments, Z is

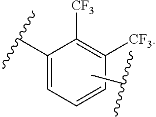

In some embodiments, Z is

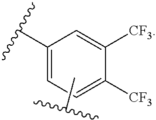

In some embodiments, Z is

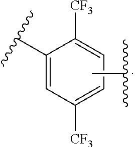

In some embodiments, Z is

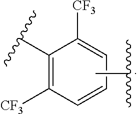

In some embodiments, Z is

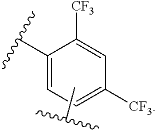

In some embodiments, Z is

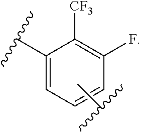

In some embodiments, Z is

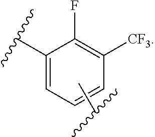

In some embodiments, Z is

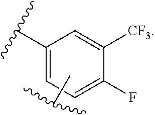

In some embodiments, Z is

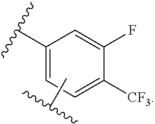

In some embodiments, Z is

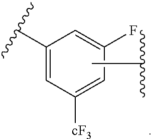

In some embodiments, Z is

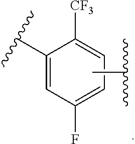

In some embodiments, Z is

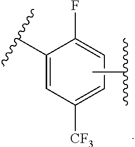

In some embodiments, Z is

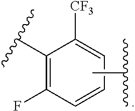

In some embodiments, Z is

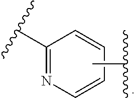

In some embodiments, Z is

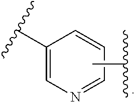

In some embodiments, Z is

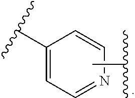

In some embodiments, Z is

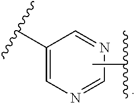

In some embodiments, Z is

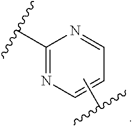

With respect to any relevant structural representation, such as Formula 1, 1C, or 1T, R⁶ is H; optionally substituted C₁₋₁₂ hydrocarbon group, including optionally substituted C₁₋₁₂ alkyl, such as optionally substituted branched C₂₋₁₂ alkyl or optionally substituted linear C₁₋₁₂ alkyl, including optionally substituted branched C₂₋₆ alkyl or linear C₁₋₆ alkyl, optionally substituted branched C₃ alkyl (e.g., —C(CH₃)₂), or linear C₁₋₃ alkyl (e.g., —CH₃, —C₂H₅, —C₃H₇), optionally substituted branched, linear, or cyclic C₃₋₆ alkyl (e.g. —C₃H₇, —C₄H₉, —C₅H₁₁, —C₆H₁₃, —C(CH₃)₂, —CH(CH₃)(CH₂CH₃), —CH(CH₂CH₃)₂, —CH(CH₃)(CH₂CH₂CH₃), —CH(CH₃)(CH₂CH₂CH₂CH₃), —CH(CH₂CH₃)(CH₂CH₂CH₃), cyclopropyl, cyclobutyl, cyclopentyl, cyclohexyl, etc.), optionally substituted branched, linear, or cyclic C₆₋₉ alkyl (e.g., —CH(CH₃)(CH₂CH₂CH₂CH₃), —CH(CH₂CH₃)(CH₂CH₂CH₃), —C₆H₁₃, —C₇H₁₅, —C₈H₁₇, —C₉H₁₉, cyclohexyl, cycloheptyl, cyclooctyl, cyclononyl, etc.), optionally substituted branched, linear, or cyclic C₉₋₁₂ alkyl, C₂₋₁₂ alkenyl, C₂₋₁₂ alkynyl, optionally substituted benzyl, etc.; optionally substituted carbocycle, including optionally substituted C₃₋₁₂ cycloalkyl, optionally substituted C₃₋₆ cycloalkyl, optionally substituted C₆₋₉ cycloalkyl, optionally substituted C₉₋₁₂ cycloalkyl, optionally substituted C₃₋₁₂ cycloalkenyl, optionally substituted C₃₋₆ cycloalkenyl, optionally substituted C₆₋₉ cycloalkenyl, optionally substituted C₉₋₁₂ cycloalkenyl, optionally substituted C₃₋₁₂ cycloalkynyl, optionally substituted C₃₋₆ cycloalkynyl, optionally substituted C₆₋₉ cycloalkynyl, optionally substituted C₉₋₁₂ cycloalkynyl, optionally substituted phenyl, optionally substituted naphthyl, or optionally substituted heterocycle such as an optionally substituted monocyclic heterocycle having 3 ring carbon atoms and 1 ring oxygen atom, an optionally substituted monocyclic heterocycle having 4 ring carbon atoms and 1 ring oxygen atom, an optionally substituted monocyclic heterocycle having 5 ring carbon atoms and 1 ring oxygen atom, an optionally substituted monocyclic heterocycle having 6 ring carbon atoms and 1 ring oxygen atom, an optionally substituted monocyclic heterocycle having 7 ring carbon atoms and 1 ring oxygen atom, an optionally substituted monocyclic heterocycle having 3 ring carbon atoms and 1 ring sulfur atom, an optionally substituted monocyclic heterocycle having 4 ring carbon atoms and 1 ring sulfur atom, an optionally substituted monocyclic heterocycle having 5 ring carbon atoms and 1 ring sulfur atom, an optionally substituted monocyclic heterocycle having 6 ring carbon atoms and 1 ring sulfur atom, an optionally substituted monocyclic heterocycle having 7 ring carbon atoms and 1 ring sulfur atom, an optionally substituted monocyclic heterocycle having 3 ring carbon atoms and 1 ring nitrogen atom, an optionally substituted monocyclic heterocycle having 4 ring carbon atoms and 1 ring nitrogen atom, an optionally substituted monocyclic heterocycle having 5 ring carbon atoms and 1 ring nitrogen atom, an optionally substituted monocyclic heterocycle having 6 ring carbon atoms and 1 ring nitrogen atom, an optionally substituted monocyclic heterocycle having 7 ring carbon atoms and 1 ring nitrogen atom, an optionally substituted monocyclic heterocycle having 3 ring carbon atoms and 2 ring heteroatoms (N, O, and/or S), an optionally substituted monocyclic heterocycle having 4 ring carbon atoms and 2 ring heteroatoms (N, O, and/or S), an optionally substituted monocyclic heterocycle having 5 ring carbon atoms and 2 ring heteroatoms (N, O, and/or S), an optionally substituted monocyclic heterocycle having 6 ring carbon atoms and 2 ring heteroatoms (N, O, and/or S), an optionally substituted monocyclic heterocycle having 2 ring carbon atoms and 3 ring heteroatoms (N, O, and/or S), an optionally substituted monocyclic heterocycle having 3 ring carbon atoms and 3 ring heteroatoms (N, O, and/or S), an optionally substituted monocyclic heterocycle having 4 ring carbon atoms and 3 ring heteroatoms (N, O, and/or S), an optionally substituted monocyclic heterocycle having 5 ring carbon atoms and 3 ring heteroatoms (N, O, and/or S), an optionally substituted bicyclic heterocycle having 6 ring carbon atoms and 1 ring heteroatom (N, O, or S), an optionally substituted bicyclic heterocycle having 7 ring carbon atoms and 1 ring heteroatom (N, O, or S), an optionally substituted bicyclic heterocycle having 8 ring carbon atoms and 1 ring heteroatom (N, O, or S), an optionally substituted bicyclic heterocycle having 9 ring carbon atoms and 1 ring heteroatom (N, O, or S), an optionally substituted bicyclic heterocycle having 10 ring carbon atoms and 1 ring heteroatom (N, O, or S), an optionally substituted bicyclic heterocycle having 11 ring carbon atoms and 1 ring heteroatom (N, O, or S), an optionally substituted bicyclic heterocycle having 12 ring carbon atoms and 1 ring heteroatom (N, O, or S), an optionally substituted bicyclic heterocycle having 5 ring carbon atoms and 2 ring heteroatoms (N, O, and/or S), an optionally substituted bicyclic heterocycle having 6 ring carbon atoms and 2 ring heteroatoms (N, O, and/or S), an optionally substituted bicyclic heterocycle having 7 ring carbon atoms and 2 ring heteroatoms (N, O, and/or S), an optionally substituted bicyclic heterocycle having 8 ring carbon atoms and 2 ring heteroatoms (N, O, and/or S), an optionally substituted bicyclic heterocycle having 9 ring carbon atoms and 2 ring heteroatoms (N, O, and/or S), an optionally substituted bicyclic heterocycle having 10 ring carbon atoms and 2 ring heteroatoms (N, O, and/or S), an optionally substituted bicyclic heterocycle having 11 ring carbon atoms and 2 ring heteroatoms (N, O, and/or S), an optionally substituted bicyclic heterocycle having 5 ring carbon atoms and 3 ring heteroatoms (N, O, and/or S), an optionally substituted bicyclic heterocycle having 6 ring carbon atoms and 3 ring heteroatoms (N, O, and/or S), an optionally substituted bicyclic heterocycle having 7 ring carbon atoms and 3 ring heteroatoms (N, O, and/or S), an optionally substituted bicyclic heterocycle having 8 ring carbon atoms and 3 ring heteroatoms (N, O, and/or S), an optionally substituted bicyclic heterocycle having 9 ring carbon atoms and 3 ring heteroatoms (N, O, and/or S), an optionally substituted bicyclic heterocycle having 10 ring carbon atoms and 3 ring heteroatoms (N, O, and/or S), optionally substituted oxetane, optionally substituted tetrahydrofuran, optionally substituted dihydrofuran, optionally substituted furan, optionally substituted furanone, optionally substituted tetrahydropyran, optionally substituted dihydropyran, an optionally substituted pyran, optionally substituted tetrahydropyrone, optionally substituted dihydropyrone, optionally substituted pyrone, optionally substituted thietane, optionally substituted tetrahydrothiophene, optionally substituted dihydrothiophene, an optionally substituted thiophene, optionally substituted azetidine, optionally substituted pyrrolidine, optionally substituted pyrroline, optionally substituted pyrrole, optionally substituted piperidine, optionally substituted pyridine, optionally substituted oxazole, optionally substituted isoxazole, optionally substituted thiazole, optionally substituted isothiazole, optionally substituted pyrazolidine, optionally substituted imidazolidine, optionally substituted pyrazole, optionally substituted imidazole, optionally substituted tetrazole, optionally substituted sulfolane.

Potential substituents on R₆ include alkyl, such as C₁₋₁₂ alkyl, C₁₋₃ alkyl, C₃₋₆ alkyl, C₆₋₉ alkyl, C₉₋₁₂ alkyl, CH₃, —C₂H₅, —C₃H₇, —C₄H₉, —C₅H₁₁, —C₆H₁₃, —C₇H₁₅, —C₈H₁₇, —C₉H₁₉, —C₁₀H₂₁, —C₁₁H₂₃, —C₁₂H₂₅, etc.; halo, such as F, Cl, Br, I, etc.; OH; —CO₂H; acyl, such as C₁₋₁₂—C(═O)-alkyl, C₁₋₃—C(═O)-alkyl, C₃₋₆—C(═O)-alkyl, C₆₋₉—C(═O)-alkyl, C₉₋₁₂—C(═O)-alkyl, —C(═O)—CH₃, —C(═O)—C₂H₅, —C(═O)—C₃H₇, —C(═O)—C₄H₉, —C(═O)—C₅H₁₁, —C(═O)—C₆H₁₃, —C(═O)—C₇H₁₅, —C(═O)—C₈H₁₇, —C(═O)—C₉H₁₉, —C(═O)—C₁₀H₂₁, —C(═O)—C₁₁H₂₃, —C(═O)—C₁₂H₂₅, —C(═O)-phenyl, etc.; alkoxy, such as such as C₁₋₁₂—O-alkyl, C₁₋₃—O-alkyl, C₃₋₆—O-alkyl, C₆₋₉—O-alkyl, C₉₋₁₂—O-alkyl, —OCH₃, —OC₂H₅, —OC₃H₇, —OC₄H₉, —OC₅H₁₁, —OC₆H₁₃, —OC₇H₁₅, —OC₈H₁₇, —OC₉H₁₉, —OC₁₀H₂₁, —OC₁₁H₂₃, —OC₁₂H₂₅, etc.; alkylthio, such as C₁₋₁₂—S-alkyl, C₁₋₃—S-alkyl, C₃₋₆—S-alkyl, C₆₋₉—S-alkyl, C₉₋₁₂—S-alkyl, —SCH₃, —SC₂H₅, —SC₃H₇, —SC₄H₉, —SC₅H₁₁, —SC₆H₁₃, —SC₇H₁₅, —SC₈H₁₇, —SC₉H₁₉, —SC₁₀H₂₁, —SC₁₁H₂₃, —SC₁₂H₂₅, etc.; thioester (e.g. —C(O)SR^{A}, —SC(O)R^{A}, etc.); phosphoryl; amino (e.g. NR^{A}R^{B}, where NR^{A}R^{B} may potentially form a ring, or N⁺R^{A}R^{B}R^{C}, wherein R^{C} is H or hydrocarbyl, and N⁺R^{A}R^{B}R^{C} may potentially form a ring); amide (e.g. —C(═O)NR^{A}R^{B}, including where NR^{A}R^{B} form a ring); CN; —NO₂; azido; alkenyl, such as C₂₋₁₂ alkenyl, C₂₋₄ alkenyl, C₄₋₆ alkenyl, C₆₋₈ alkenyl, C₈₋₁₀ alkenyl, C₁₀₋₁₂ alkenyl, (e.g. —CH═CH₂, —CH═CH₂CH₃, etc.); alkynyl, such as C₂₋₁₂ alkynyl, C₂₋₄ alkynyl, C₄₋₆ alkynyl, C₆₋₈ alkynyl, C₈₋₁₀ alkynyl, C₁₀₋₁₂ alkynyl, (e.g. —C═CH, —C═C—CH₃, etc.); cycloalkyl, such as cyclopropyl, cyclobutyl, cyclopentyl, cyclohexyl, cyclohexyl, etc.; heterocyclylalkyl (e.g. -alkyl-heterocycle); heteroaralkyl (e.g. -alkyl-heteroaryl); sulfonamide (e.g. —SO₂NR^{A}R^{B}, —NR^{A}SO₂R^{B}, etc.); aryl; heteroaryl; heterocyclyl; aralkyl (e.g. -alkyl-aryl; etc. In some embodiments, R⁶ is H. In some embodiments, R⁶ is C₁₋₁₂ alkyl. In some embodiments, R⁶ is linear C₁₋₁₂ alkyl. In some embodiments, R⁶ is —CH₃, —C₂H₅, —C₃H₇, —C₄H₉, —C₅H₁₁, —C₆H₁₃, —C₇H₁₅, —C₈H₁₇, —C₉H₁₉, —C₁₀H₂₁, —C₁₁H₂₃, —C₁₂H₂₅. In some embodiments, R⁶ is an optionally substituted linear C₁₋₁₂ alkyl. In some embodiments, R⁶ is branched C₃₋₁₂ alkyl. In some embodiments, R⁶ is an optionally substituted branched C₃₋₁₂ alkyl. In some embodiments, R⁶ is an optionally substituted carbocycle, such as a carbocycle having electron-withdrawing substituents including acyl groups (e.g., —C(O)R, etc.) esters (e.g., —CO₂R, etc.), amides (e.g., —C(O)NR₂, etc.), imides (e.g., —C(O)NRC(O)R, etc.), cyano (—CN), sulfones (e.g., —SO₂R, etc.), sulfonamides (e.g., —SO₂NR₂), fluorine or fluorine containing groups (e.g., F, CF₃, CF₂CF₃, CHF₂, CH₂F, CF₂CF₂CF₃, etc.), and/or nitro (—NO₂). In some aspects, R⁶ is an electron-deficient heterocyclic moiety. In some aspects, R⁶ is an electron-deficient aryl moiety. In some aspects, R⁶ is an electron-deficient alkyl moiety.

In some embodiments, R⁶ is an optionally substituted carbocycle. In some embodiments, R⁶ is optionally substituted cyclohexyl. In some embodiments, R⁶ is an optionally substituted aryl. In some embodiments, R⁶ is an optionally substituted phenyl. In some embodiments, R⁶ is an optionally substituted benzyl. In some embodiments, R⁶ is an optionally substituted heteroaryl. In some embodiments, R⁶ is an optionally substituted heterocycle. In some embodiments wherein R⁶ is an optionally substituted heterocycle, a carbon atom of the heterocycle (rather than a heteroatom of the heterocycle) is directly attached to Z.

In some embodiments, R⁶ is fluoro substituted C₁₋₆ alkyl, including C₁₋₆ perfluoralkyl. In some embodiments, R⁶ is fluoro substituted branched C₂₋₆ alkyl, such as branched C₂₋₆ perfluoroalkyl. In some embodiments, R⁶ is —CF₃, —C₂F₅, —C₃F₇, —C₄F₉, —C₅F₁₁₀, or —C₆F₁₃. In some embodiments, R⁶ is —CF₃. In some embodiments, R⁶ is —C₂F₅. In some embodiments, R⁶ is —C₃F₇. In some embodiments, R⁶ is —C₄F₉. In some embodiments, R⁶ is —C₅F₁₁. In some embodiments, R⁶ is —C₆F₁₃. In some embodiments, R⁶ is CF₃. In some embodiments, R⁶ is CHF₂. In some embodiments, R⁶ is CH₂F. In some embodiments, R⁶ is CF₂CF₃. In some embodiments, R⁶ is CF₂CF₂CF₃. In some embodiments, R⁶ is fluoro substituted isopropyl, including perfluoroisopropyl. In some embodiments, R⁶ is fluoro substituted isobutyl, including perfluoroisobutyl. In some embodiments, R⁶ is fluoro substituted tert-butyl including perfluoro-tert-butyl. In some embodiments, R⁶ is CF₃. In some embodiments, R⁶ is CHF₂. In some embodiments, R⁶ is CH₂F. In some embodiments, R⁶ is CF₂CF₃. In some embodiments, R⁶ is CF₂CF₂CF₃.

In some embodiments, R⁶ is an optionally substituted oxetane. In some embodiments, R⁶ is an optionally substituted oxetane having a carbon atom of the oxetane ring directly attached to Z.

In some embodiments, R⁶ is an optionally substituted tetrahydrofuran. In some embodiments, R⁶ is an optionally substituted tetrahydrofuran having a carbon atom of the tetrahydrofuran ring directly attached to Z.

In some embodiments, R⁶ is an optionally substituted dihydrofuran. In some embodiments, R⁶ is an optionally substituted dihydrofuran having a carbon atom of the dihydrofuran ring directly attached to Z.

In some embodiments, R⁶ is an optionally substituted furan. In some embodiments, R⁶ is an optionally substituted furan having a carbon atom of the furan ring directly attached to Z.

In some embodiments, R⁶ is an optionally substituted furanone. In some embodiments, R⁶ is an optionally substituted furanone having a carbon atom of the furanone ring directly attached to Z.

In some embodiments, R⁶ is an optionally substituted tetrahydropyran. In some embodiments, R⁶ is an optionally substituted tetrahydropyran having a carbon atom of the tetrahydropyran ring directly attached to Z.

In some embodiments, R⁶ is an optionally substituted dihydropyran. In some embodiments, R⁶ is an optionally substituted dihydropyran having a carbon atom of the dihydropyran ring directly attached to Z.

In some embodiments, R⁶ is an optionally substituted pyran. In some embodiments, R⁶ is an optionally substituted pyran having a carbon atom of the pyran ring directly attached to Z.

In some embodiments, R⁶ is an optionally substituted tetrahydropyrone. In some embodiments, R⁶ is an optionally substituted tetrahydropyrone having a carbon atom of the tetrahydropyrone ring directly attached to Z.

In some embodiments, R⁶ is an optionally substituted dihydropyrone. In some embodiments, R⁶ is an optionally substituted dihydropyrone having a carbon atom of the dihydropyrone ring directly attached to Z.

In some embodiments, R⁶ is an optionally substituted pyrone. In some embodiments, R⁶ is an optionally substituted pyrone having a carbon atom of the pyrone ring directly attached to Z.

In some embodiments, R⁶ is an optionally substituted thietane. In some embodiments, R⁶ is an optionally substituted thietane having a carbon atom of the thietane ring directly attached to Z.

In some embodiments, R⁶ is an optionally substituted tetrahydrothiophene. In some embodiments, R⁶ is an optionally substituted tetrahydrothiophene having a carbon atom of the tetrahydrothiophene ring directly attached to Z.

In some embodiments, R⁶ is an optionally substituted dihydrothiophene. In some embodiments, R⁶ is an optionally substituted dihydrothiophene having a carbon atom of the dihydrothiophene ring directly attached to Z.

In some embodiments, R⁶ is an optionally substituted thiophene. In some embodiments, R⁶ is an optionally substituted thiophene having a carbon atom of the thiophene ring directly attached to Z.

In some embodiments, R⁶ is an optionally substituted azetidine. In some embodiments, R⁶ is an optionally substituted azetidine having a carbon atom of the azetidine ring directly attached to Z. In some embodiments, R⁶ is azetidine having an optionally substituted diphenylmethyl substituent. In some embodiments, R⁶ is azetidine having an optionally substituted diphenylmethyl substituent attached to the nitrogen atom of the azetidine ring.

In some embodiments, R⁶ is an optionally substituted pyrrolidine. In some embodiments, R⁶ is an optionally substituted pyrrolidine having a carbon atom of the pyrrolidine ring directly attached to Z.

In some embodiments, R⁶ is an optionally substituted pyrroline. In some embodiments, R⁶ is an optionally substituted pyrroline having a carbon atom of the pyrroline ring directly attached to Z.

In some embodiments, R⁶ is an optionally substituted pyrrole. In some embodiments, R⁶ is an optionally substituted pyrrole having a carbon atom of the pyrrole ring directly attached to Z.

In some embodiments, R⁶ is an optionally substituted piperidine. In some embodiments, R⁶ is an optionally substituted piperidine having a carbon atom of the piperidine ring directly attached to Z.

In some embodiments, R⁶ is an optionally substituted pyridine. In some embodiments, R⁶ is an optionally substituted pyridine having a carbon atom of the pyridine ring directly attached to Z.

In some embodiments, R⁶ is an optionally substituted oxazole. In some embodiments, R⁶ is an optionally substituted oxazole having a carbon atom of the oxazole ring directly attached to Z.

In some embodiments, R⁶ is an optionally substituted isoxazole. In some embodiments, R⁶ is an optionally substituted isoxazole having a carbon atom of the isoxazole ring directly attached to Z.

In some embodiments, R⁶ is an optionally substituted thiazole. In some embodiments, R⁶ is an optionally substituted thiazole having a carbon atom of the thiazole ring directly attached to Z.

In some embodiments, R⁶ is an optionally substituted isothiazole. In some embodiments, R⁶ is an optionally substituted isothiazole having a carbon atom of the isothiazole ring directly attached to Z.

In some embodiments, R⁶ is an optionally substituted pyrazolidine. In some embodiments, R⁶ is an optionally substituted pyrazolidine having a carbon atom of the pyrazolidine ring directly attached to Z.

In some embodiments, R⁶ is an optionally substituted imidazolidine. In some embodiments, R⁶ is an optionally substituted imidazolidine having a carbon atom of the imidazolidine ring directly attached to Z.

In some embodiments, R⁶ is an optionally substituted pyrazole. In some embodiments, R⁶ is an optionally substituted pyrazole having a carbon atom of the pyrazole ring directly attached to Z.

In some embodiments, R⁶ is an optionally substituted imidazole. In some embodiments, R⁶ is an optionally substituted imidazole having a carbon atom of the imidazole ring directly attached to Z.

In some embodiments, R⁶ is an optionally substituted tetrazole. In some embodiments, R⁶ is an optionally substituted tetrazole having a carbon atom of the tetrazole ring directly attached to Z.

In some embodiments, R⁶ is an optionally substituted sulfolane. In some embodiments, R⁶ is an optionally substituted sulfolane having a carbon atom of the sulfolane ring directly attached to Z.

In some embodiments, R⁶ is an optionally substituted phenyl. In some embodiments, R⁶ is represented by one of the following structures:

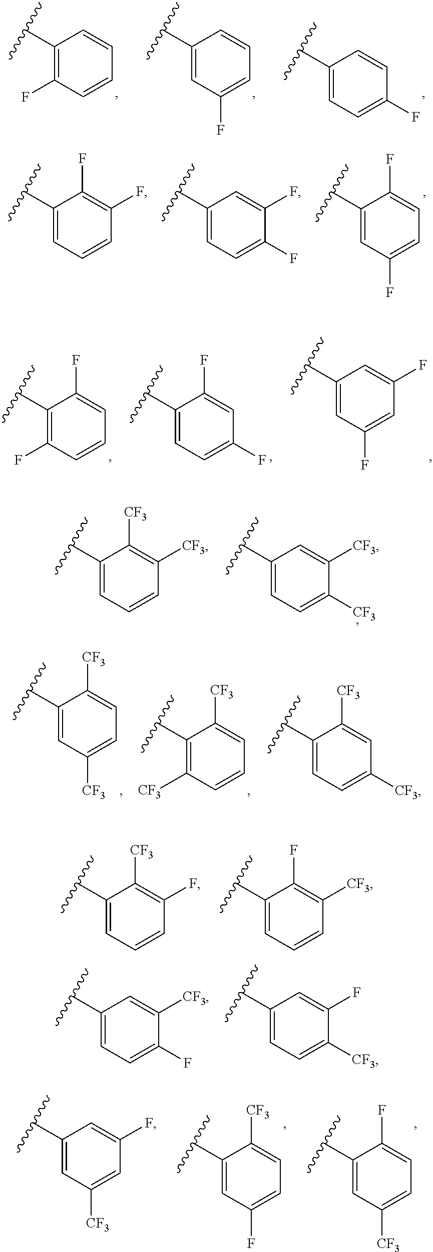

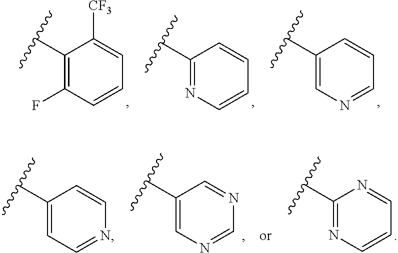

In some embodiments, R⁶ is

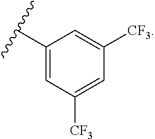 [SMILES: *c1cc(C(F)(F)F)cc(C(F)(F)F)c1]

In some embodiments, R⁶ is not

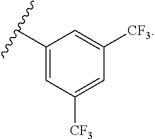 [SMILES: *c1cc(C(F)(F)F)cc(C(F)(F)F)c1]

In some embodiments, R⁶ is

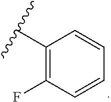 [SMILES: *c1ccccc1F]

In some embodiments, R⁶ is

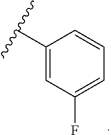 [SMILES: *c1cccc(F)c1]

In some embodiments, R⁶ is

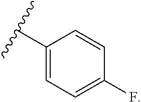 [SMILES: *c1ccc(F)cc1]

In some embodiments, R⁶ is

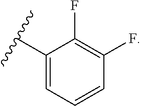 [SMILES: *c1cccc(F)c1F]

In some embodiments, R⁶ is

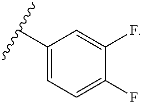 [SMILES: *c1ccc(F)c(F)c1]

In some embodiments, R⁶ is

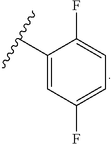 [SMILES: *c1cc(F)ccc1F]

In some embodiments, R⁶ is

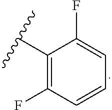 [SMILES: *c1c(F)cccc1F]

In some embodiments, R⁶ is

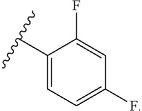 [SMILES: *c1ccc(F)cc1F]

In some embodiments, R⁶ is

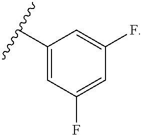 [SMILES: *c1cc(F)cc(F)c1]

In some embodiments, R⁶ is

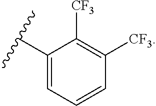 [SMILES: *c1cccc(C(F)(F)F)c1C(F)(F)F]

In some embodiments, R⁶ is

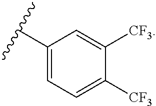 [SMILES: *c1ccc(C(F)(F)F)c(C(F)(F)F)c1]

In some embodiments, R⁶ is

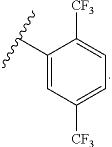 [SMILES: *c1cc(C(F)(F)F)ccc1C(F)(F)F]

In some embodiments, R⁶ is

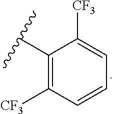 [SMILES: *c1c(C(F)(F)F)cccc1C(F)(F)F]

In some embodiments, R⁶ is

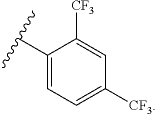 [SMILES: *c1ccc(C(F)(F)F)cc1C(F)(F)F]

In some embodiments, R⁶ is

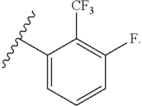 [SMILES: *c1cccc(F)c1C(F)(F)F]

In some embodiments, R⁶ is

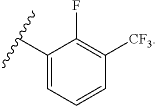 [SMILES: *c1cccc(C(F)(F)F)c1F]

In some embodiments, R⁶ is

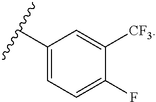 [SMILES: *c1ccc(F)c(C(F)(F)F)c1]

In some embodiments, R⁶ is

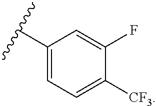 [SMILES: *c1ccc(C(F)(F)F)c(F)c1]

In some embodiments, R⁶ is

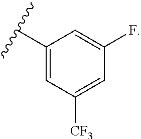 [SMILES: *c1cc(F)cc(C(F)(F)F)c1]

In some embodiments, R⁶ is

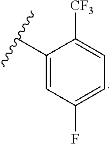 [SMILES: *c1cc(F)ccc1C(F)(F)F]

In some embodiments, R⁶ is

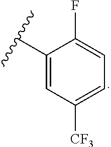 [SMILES: *c1cc(C(F)(F)F)ccc1F]

In some embodiments, R⁶ is

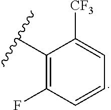 [SMILES: *c1c(F)cccc1C(F)(F)F]

In some embodiments, R⁶ is

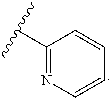 [SMILES: *c1ccccn1]

In some embodiments, R⁶ is

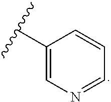 [SMILES: *c1cccnc1]

In some some embodiments, R⁶ is

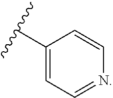 [SMILES: *c1ccncc1]

In some embodiments, R⁶ is

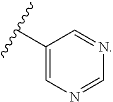 [SMILES: *c1cncnc1]

In some embodiments, R⁶ is

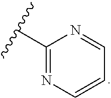 [SMILES: *c1ncccn1]

In some embodiments, Z is — and R⁶ is an optionally substituted aryl.

In some embodiments, Z is — and R⁶ is an optionally substituted alkyl.

With respect to any relevant structural representation, such as Formula 1, R¹ is H, F, Cl, Br, I, OH, OR^{A}, SH, SR^{A}, NH₂, NHR^{A}, NR^{A}R^{B}, CF₃, CN, carboxylic acid (CO₂H), optionally substituted carboxylic ester (such as optionally substituted C₁₋₆ alkyl carboxylic ester, such as optionally substituted branched C₂₋₆ alkyl or linear C₁₋₆ alkyl carboxylic ester, optionally substituted branched C₃ alkyl carboxylic ester (e.g., —CO₂—C(CH₃)₂), or linear C₁₋₃ alkyl carboxylic ester (e.g., —CO₂—CH₃, —CO₂—C₂H₅, —CO₂—C₃H₇), optionally substituted branched, linear, or cyclic C₃₋₆ alkyl carboxylic ester (e.g. —CO₂—C₃H₇, —CO₂—C₄H₉, —CO₂—C₅H₁₁, —CO₂—C₆H₁₃, —CO₂—CH(CH₃)₂, —CO₂—CH(CH₃)(CH₂CH₃), —CO₂—CH(CH₂CH₃)₂, —CO₂—CH(CH₃)(CH₂CH₂CH₃), —CO₂—C(CH₃)₂(CH₂CH₂CH₃), —CO₂—C(CH₃)(CH₂CH₂CH₂CH₃), —CO₂—CH(CH₂CH₃)(CH₂CH₂CH₃), —CO₂-cyclopropyl, —CO₂— cyclobutyl, —CO₂-cyclopentyl, —CO₂-cyclohexyl, etc.), or optionally substituted C₁₋₆ alkyl, such as optionally substituted branched C₂₋₆ alkyl or linear C₁₋₆ alkyl, optionally substituted branched C₃ alkyl (e.g., —C(CH₃)₂), or linear C₁₋₃ alkyl (e.g., —CH₃, —C₂H₅, —C₃H₇), optionally substituted branched, linear, or cyclic C₃₋₆ alkyl (e.g. —C₃H₇, —C₄H₉, —C₅H₁₁, —C₆H₁₃, —CH(CH₃)₂, —CH(CH₃)(CH₂CH₃), —CH(CH₂CH₃)₂, —CH(CH₃)(CH₂CH₂CH₃), —C(CH₃)₂(CH₂CH₂CH₃), —C(CH₃)(CH₂CH₂CH₂CH₃), —CH(CH₂CH₃)(CH₂CH₂CH₃), cyclopropyl, cyclobutyl, cyclopentyl, cyclohexyl, etc.).

In some embodiments, R¹ is H. In some embodiments, R⁷ is F. In some embodiments, R⁷ is C₁. In some embodiments, R⁷ is Br. In some embodiments, R⁷ is I. In some embodiments, R⁷ is OH. In some embodiments, R⁷ is OR^{A}. In some embodiments, R⁷ is SH. In some embodiments, R⁷ is SR^{A}. In some embodiments, R⁷ is NH₂. In some embodiments, R⁷ is NHR^{A}. In some embodiments, R⁷ is NR^{A}R^{B}. In some embodiments, R⁷ is CF₃. In some embodiments, R⁷ is CN. In some embodiments, R⁷ is CO₂H. In some embodiments, R⁷ is CO₂R^{A}. In some embodiments, R⁷ is C₁₋₆ alkyl. In some embodiments, R⁷ is branched C₂₋₆ alkyl. In some embodiments, R⁷ is —CH₃, —C₂H₅, —C₃H₇, —C₄H₉, —C₅H₁₁, or —C₆H₁₃. In some embodiments, R⁷ is an optionally substituted C₁₋₆ alkyl. In some embodiments, R⁷ is an optionally heteroatom substituted linear C₁₋₆ alkyl, such as a linear C₁₋₆ alkyl having polar substituents, including oxygen containing groups (e.g. —OH, =O, OCH₃, etc.), sulfur containing groups (e.g. —SH, —SCH₃, SO₂, SO₃⁻, etc.), nitrogen containing groups (e.g. —NH₂, —NHCH₃, —N(CH₃)₂, —NO₂, —CN, etc.), fluorine containing groups (F, CF₃, CF₂CF₃, CHF₂, CH₂F, CF₂CF₂CF₃, etc.). In some embodiments, R⁷ is an optionally substituted branched C₂₋₆ alkyl. In some embodiments, R⁷ is an optionally heteroatom substituted branched C₂₋₆ alkyl, such as a branched C₂₋₆ alkyl having polar substituents, including oxygen containing groups (e.g. —OH, ═O, OCH₃, etc.), sulfur containing groups (e.g. —SH, —SCH₃, SO₂, SO₃⁻, etc.), nitrogen containing groups (e.g. —NH₂, —NHCH₃, —N(CH₃)₂, —NO₂, —CN, etc.), fluorine containing groups (F, CF₃, CF₂CF₃, CHF₂, CH₂F, CF₂CF₂CF₃, etc.).

In some embodiments, the compound is a compound shown below, each of which may be optionally substituted:

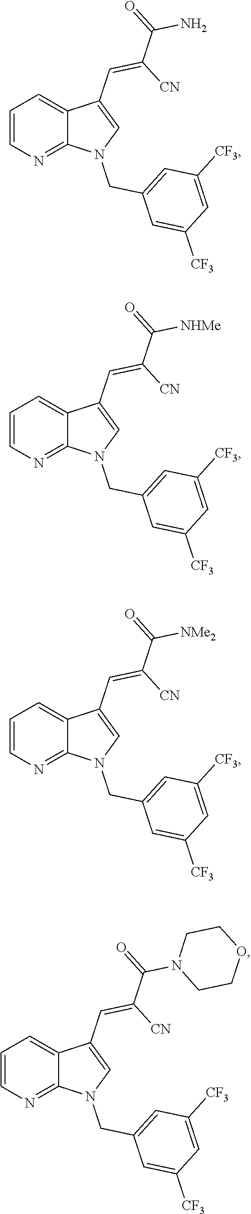 [SMILES: N#C/C(=C\c1cn(Cc2cc(C(F)(F)F)cc(C(F)(F)F)c2)c2ncccc12)C(N)=O ; CNC(=O)/C(C#N)=C/c1cn(Cc2cc(C(F)(F)F)cc(C(F)(F)F)c2)c2ncccc12 ; CN(C)C(=O)/C(C#N)=C/c1cn(Cc2cc(C(F)(F)F)cc(C(F)(F)F)c2)c2ncccc12 ; N#C/C(=C\c1cn(Cc2cc(C(F)(F)F)cc(C(F)(F)F)c2)c2ncccc12)C(=O)N1CCOCC1]

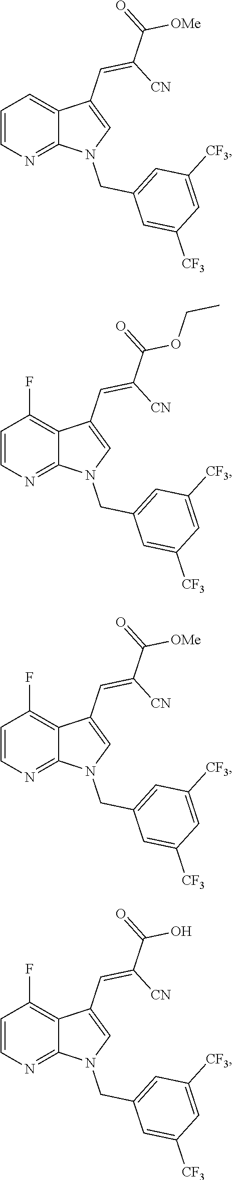 [SMILES: COC(=O)/C(C#N)=C/c1cn(Cc2cc(C(F)(F)F)cc(C(F)(F)F)c2)c2ncccc12 ; CCOC(=O)/C(C#N)=C/c1cn(Cc2cc(C(F)(F)F)cc(C(F)(F)F)c2)c2nccc(F)c12 ; COC(=O)/C(C#N)=C/c1cn(Cc2cc(C(F)(F)F)cc(C(F)(F)F)c2)c2nccc(F)c12 ; N#C/C(=C\c1cn(Cc2cc(C(F)(F)F)cc(C(F)(F)F)c2)c2nccc(F)c12)C(=O)O]

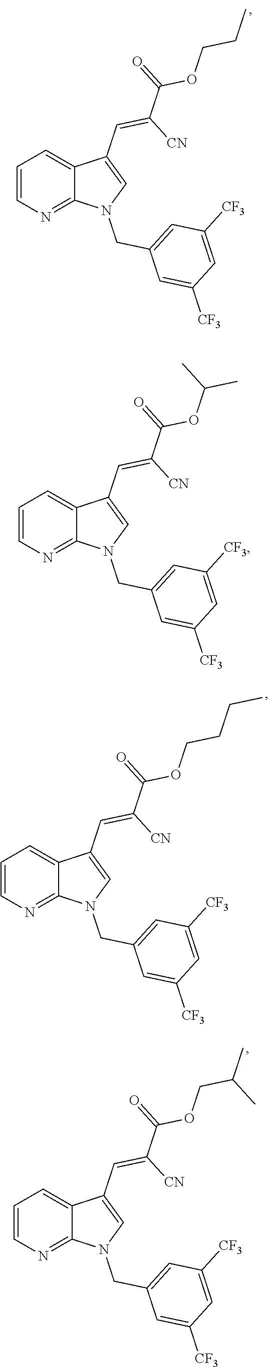 [SMILES: CCCOC(=O)/C(C#N)=C/c1cn(Cc2cc(C(F)(F)F)cc(C(F)(F)F)c2)c2ncccc12 ; CC(C)OC(=O)/C(C#N)=C/c1cn(Cc2cc(C(F)(F)F)cc(C(F)(F)F)c2)c2ncccc12 ; CCCCOC(=O)/C(C#N)=C/c1cn(Cc2cc(C(F)(F)F)cc(C(F)(F)F)c2)c2ncccc12 ; CC(C)COC(=O)/C(C#N)=C/c1cn(Cc2cc(C(F)(F)F)cc(C(F)(F)F)c2)c2ncccc12]

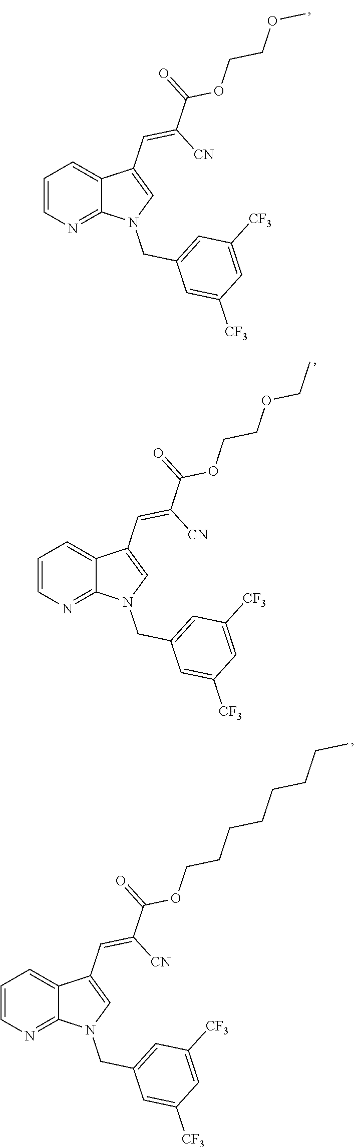 [SMILES: COCCOC(=O)/C(C#N)=C/c1cn(Cc2cc(C(F)(F)F)cc(C(F)(F)F)c2)c2ncccc12 ; CCOCCOC(=O)/C(C#N)=C/c1cn(Cc2cc(C(F)(F)F)cc(C(F)(F)F)c2)c2ncccc12 ; CCCCCCCCOC(=O)/C(C#N)=C/c1cn(Cc2cc(C(F)(F)F)cc(C(F)(F)F)c2)c2ncccc12]

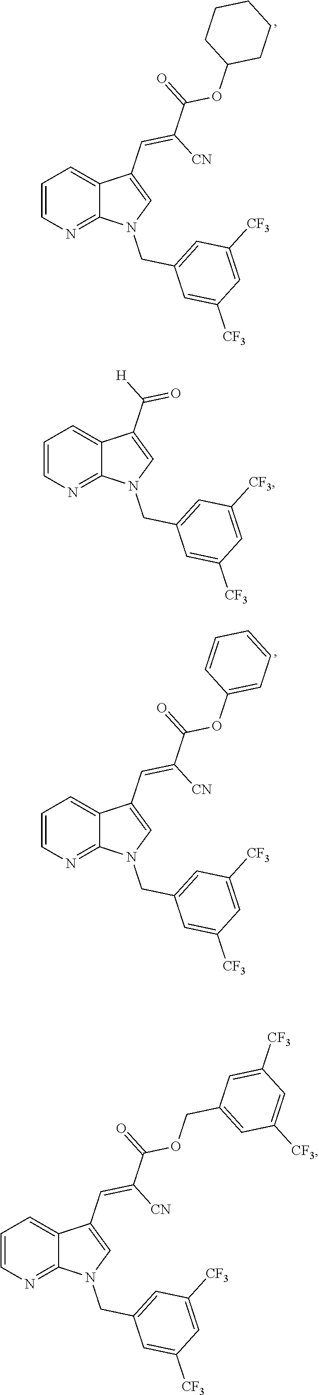

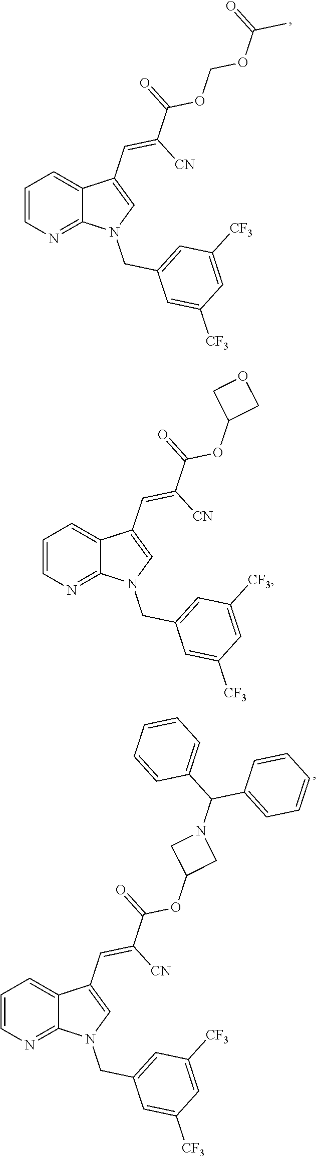 [SMILES: CC(=O)OCOC(=O)/C(C#N)=C/c1cn(Cc2cc(C(F)(F)F)cc(C(F)(F)F)c2)c2ncccc12 ; N#C/C(=C\c1cn(Cc2cc(C(F)(F)F)cc(C(F)(F)F)c2)c2ncccc12)C(=O)OC1COC1 ; N#C/C(=C\c1cn(Cc2cc(C(F)(F)F)cc(C(F)(F)F)c2)c2ncccc12)C(=O)OC1CN(C(c2ccccc2)c2ccccc2)C1]

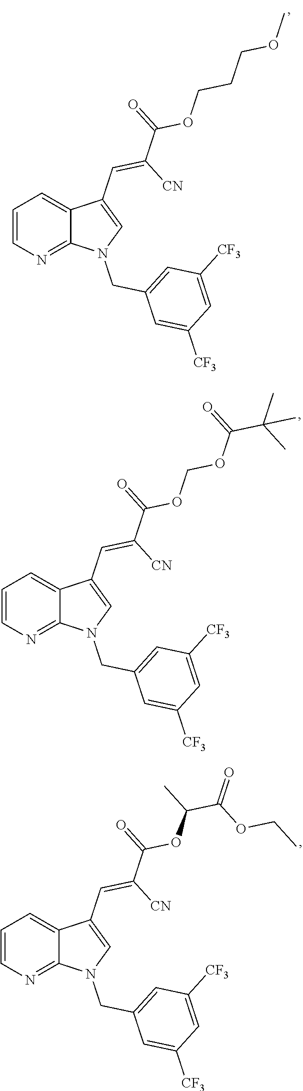

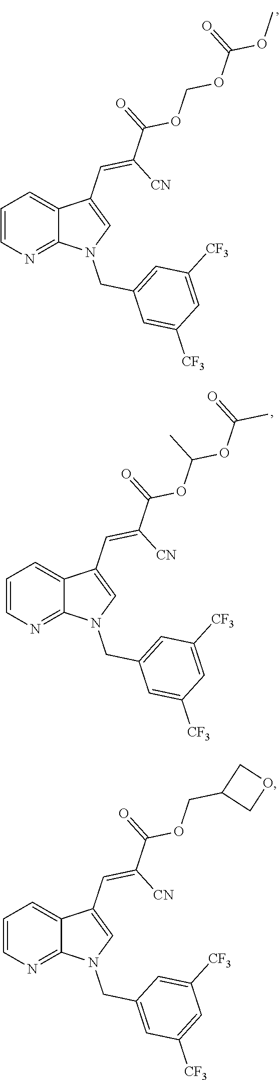

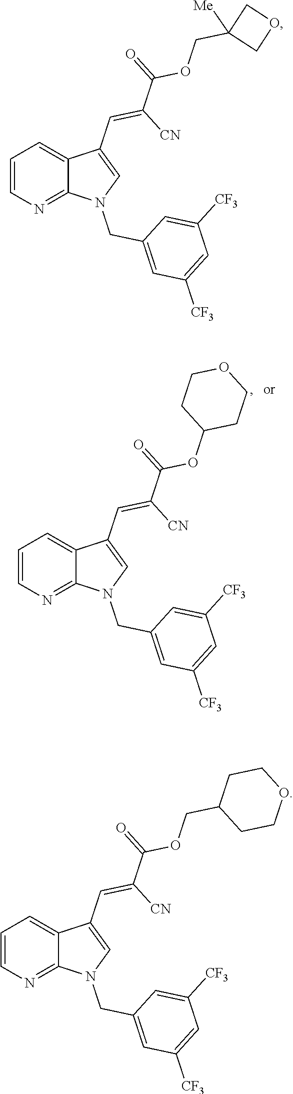 [SMILES: CC1(COC(=O)/C(C#N)=C/c2cn(Cc3cc(C(F)(F)F)cc(C(F)(F)F)c3)c3ncccc23)COC1 ; N#C/C(=C\c1cn(Cc2cc(C(F)(F)F)cc(C(F)(F)F)c2)c2ncccc12)C(=O)OC1CCOCC1 ; N#C/C(=C\c1cn(Cc2cc(C(F)(F)F)cc(C(F)(F)F)c2)c2ncccc12)C(=O)OCC1CCOCC1]

In certain aspects, the compound

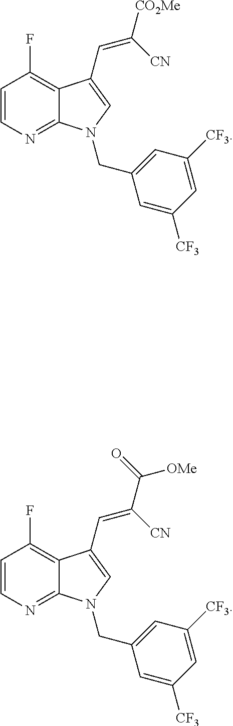 [SMILES: COC(=O)/C(C#N)=C/c1cn(Cc2cc(C(F)(F)F)cc(C(F)(F)F)c2)c2nccc(F)c12 ; COC(=O)/C(C#N)=C/c1cn(Cc2cc(C(F)(F)F)cc(C(F)(F)F)c2)c2nccc(F)c12]

In some embodiments, the compound is

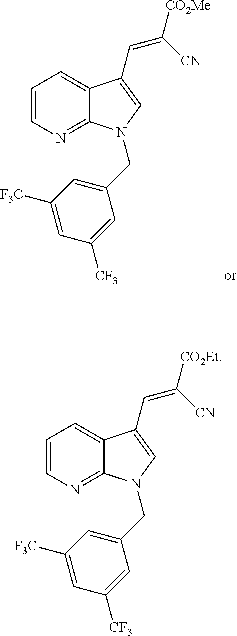 [SMILES: COC(=O)/C(C#N)=C/c1cn(Cc2cc(C(F)(F)F)cc(C(F)(F)F)c2)c2ncccc12 ; CCOC(=O)/C(C#N)=C/c1cn(Cc2cc(C(F)(F)F)cc(C(F)(F)F)c2)c2ncccc12]

The compound described herein are useful for growing hair. For example, a compound described herein may be administered to the skin of a mammal in the area where hair growth is intended.

In certain aspects, the present disclosure provides methods of enhancing lactate production in a cell, comprising contacting the cell with a compound or composition of the disclosure.

In certain aspects, the present disclosure provides methods of inhibiting mitochondrial pyruvate oxidation in a cell, comprising contacting the cell with a mitochondrial pyruvate oxidation (MPO) inhibitor, such as a compound of the present disclosure. In certain embodiments, the MPO inhibitor is a mitochondrial pyruvate carrier (MPC) inhibitor. In certain embodiments, inhibiting mitochondrial pyruvate oxidation in a cell has the effect of enhancing lactate production in a cell and/or enhancing the activity of LDH in a cell, and promoting hair growth, as described herein.

In certain aspects, the present disclosure provides methods of enhancing lactate production in a cell, comprising contacting the cell with an MPO inhibitor, such as a compound of the present disclosure. In certain embodiments, the MPO inhibitor is a mitochondrial pyruvate carrier (MPC) inhibitor.

In certain aspects, the present disclosure provides methods of enhancing the activity of LDH in a cell, comprising contacting the cell with an MPO inhibitor, such as a compound of the present disclosure. In certain embodiments, the MPO inhibitor is a mitochondrial pyruvate carrier (MPC) inhibitor.

In certain aspects, the present disclosure provides methods of enhancing the activity of lactic acid dehydrogenase (LDH) in a cell, comprising contacting the cell with an MPO inhibitor, such as a compound of the present disclosure. In certain embodiments, the MPO inhibitor is a mitochondrial pyruvate carrier (MPC) inhibitor. In certain aspects, the present disclosure provides methods of promoting hair growth or treating a hair growth condition or disorder such as baldness or alopecia, comprising administering to a patient a compound or composition as disclosed herein.

In certain aspects, the present disclosure provides methods of promoting hair growth or treating a hair growth condition or disorder such as baldness or alopecia, comprising administering to a patient an MPO inhibitor (e.g., topically, such as with a pharmaceutical composition formulated for topical application), such as a compound of the present disclosure. In certain embodiments, the present disclosure provides methods of promoting hair growth or treating a hair growth condition or disorder such as baldness or alopecia, comprising administering to a patient an MPC inhibitor (e.g., topically, such as with a pharmaceutical composition formulated for topical application), such a compound of the present disclosure. In certain embodiments, inhibiting mitochondrial pyruvate oxidation or the mitochondrial pyruvate carrier in a cell has the effect of enhancing lactate production and/or enhancing the activity of LDH in a cell, and promoting hair growth, as described herein.

For use in growing hair, a compound described herein may be mixed with a dermatologically compatible vehicle or carrier, e.g. so that the compound is present at an amount of about 0.001-10% or about 0.01-2%. The vehicle which may be employed for a topical dermatological composition may comprise, for example, aqueous solutions such as e.g., physiological salines, oil, solutions, ointments, gels, creams, sprays, etc. In some embodiments, the vehicle may contain a solvent such as ethanol or polyethylene glycol. In some embodiments, the vehicle may also contain a penetration enhancer, e.g. to enhance penetration into the skin, such as transcutol P. The vehicle furthermore may contain dermatologically compatible preservatives such as e.g., benzalkonium chloride, surfactants like e.g., polysorbate 80, liposomes or polymers, for example, methyl cellulose, polyvinyl alcohol, polyvinyl pyrrolidone and hyaluronic acid; these may be used for increasing the viscosity.

In certain aspects, the compounds of the present disclosure are mitochondrial pyruvate oxidation (MPO) inhibitors. In some embodiments, the compounds described herein may inhibit mitochondrial pyruvate carrier (MPC). In certain embodiments, the MPO inhibitor is an MPC inhibitor. In some aspects, inhibiting MPO in a cell has the effect of enhancing lactate production in a cell and/or enhancing the activity of lactic acid dehydrogenase (LDH) in a cell, and promoting hair growth. In certain aspects, the present disclosure provides methods of promoting hair growth or treating a hair growth condition or disorder such as baldness or alopecia, comprising administering to a patient an MPO inhibitor (e.g., topically, such as with a pharmaceutical composition formulated for topical application), such as a compound of the present disclosure. In certain embodiments, the present disclosure provides methods of promoting hair growth or treating a hair growth condition or disorder such as baldness or alopecia, comprising administering to a patient an MPC inhibitor (e.g., topically, such as with a pharmaceutical composition formulated for topical application), such as a compound of the present disclosure. In some embodiments, inhibiting the MPO or the MPC in a cell has the effect of enhancing lactate production and/or enhancing the activity of LDH in a cell, and promoting hair growth.

For the purposes of this disclosure, the term “treat,” “treating,” or a similar term (such as “modulating”), includes cure, mitigation, treatment, or prevention of disease in man or other animals, or any other effect that would be associated with a “drug” as defined under 21 USC 321(g).

In certain aspects, the compounds of the present disclosure may be ester prodrugs. In other aspects, the compounds described herein may be thioester or amide prodrugs. In some embodiments, the compounds herein may show a higher rate of hydrolysis (such as a rate that is at least about 1.1 times higher, at least about 1.5 times higher, at least about 2 times higher, at least about 5 times higher, at least about 10 times higher, at least about 50 times higher, at least about 100 times higher, at least about 500 times higher, at least about 1,000 times higher, at least about 10,000 times higher, about 1.1-2 times higher, about 2-4 times higher, about 4-6 times higher, about 6-8 times higher, about 8-10 times higher, about 1.1-10 times higher, about 10-100 times higher, about 100-1,000 times higher, or about 1,000-10,000 times higher) relative to conventional alkyl (ethyl or methyl) esters. In some aspects, the compounds of the present disclosure may achieve a high level of hydrolyzed drug (carboxylic acid) in skin homogenate assays.

It is understood that topical delivery of an active pharmaceutical ingredient (API) for dermal indications comprises a balance of lipophilic and hydrophilic properties. It is believed that a compound having lipophilicity as a prodrug and hydrophilicity as the corresponding free acid API may achieve the goal of reaching the desired skin layer target (e.g., a hair follicle). The rate of hydrolysis of the prodrug in the layers of the skin may be adjusted to achieve the desired result.

In some embodiments, the compounds of the present disclosure undergo hydrolysis to release the active free carboxylic acid. In some aspects, the compounds of the present disclosure undergo hydrolysis to release the active free carboxylic acid at a rate that is enhanced relative to conventional prodrugs (e.g., JXL082). In some embodiments, the rate of hydrolysis may benefit the delivery of active pharmaceutical agent to potentiate hair growth.

In some aspects, the prodrug compounds of the present disclosure undergo hydrolysis in human skin homogenate faster than known prodrugs such as JXL082. In some embodiments, the concentration of carboxylic acid (API) released by a prodrug of the present disclosure is at least about 150% greater to about 20000% greater than the amount released by a conventional prodrug such as JXL082. In some embodiments, the concentration of carboxylic acid (API) released by a prodrug of the present disclosure is at least about 150-300% greater, about 300-500% greater, about 500-1000% greater, about 1000-2000% greater, about 2000-4000% greater, about 4000-7000% greater, about 7000-10000% greater, about 10000-15000% greater, or about 15000-20000% greater, or about any value in a range bounded by any of these ranges, than the amount released by a conventional prodrug such as JXL082.

In other embodiments, the compounds of the present disclosure may enhance hair growth in their free acid form.

### Pharmaceutical Compositions

The compounds of the present disclosure may be included in a pharmaceutical and a pharmaceutically acceptable excipient.

The compositions and methods described herein may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound described herein and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound described herein. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a therapeutic compound described herein. Liposomes, for example, which comprise phospholipids or other lipids, are nontoxic, physiologically acceptable and metabolizable carriers that are relatively simple to make and administer.

The phrase “pharmaceutically acceptable” includes those compounds, materials, compositions, and/or dosage forms which would be considered to be suitable, by a person having ordinary skill in the art, for pharmaceutical use on human beings and animals.

The phrase “pharmaceutically acceptable carrier” includes a pharmaceutically acceptable material, composition or vehicle, such as a liquid or solid filler, diluent, excipient, solvent or encapsulating material. Acceptable carriers include those that are compatible with the other ingredients of the formulation and acceptable for use on the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any suitable methods. The amount of active ingredient which can be combined with a carrier material to produce a single dosage form may vary depending upon the host being treated, the particular mode of administration. The amount of active ingredient that can be combined with a carrier material to produce a single dosage form will generally be that amount of the compound which produces a therapeutic effect. Generally, out of 100%, this amount will range from about 1-99% of active ingredient, e.g. about 5-70%, about 1-10%, about 10-30%, about 30-50%, about 50-70%, about 70-99%, etc.

Methods of preparing these formulations or compositions include the step of bringing into association an active compound, such as a compound described herein, with the carrier and, optionally, one or more accessory ingredients. In general, the formulations are prepared by uniformly and intimately bringing into association a compound described herein with liquid carriers, or finely divided solid carriers, or both, and then, if necessary, shaping the product.

Formulations described herein suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound described herein as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound described herein to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” include modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

The dosage level may depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

If desired, the effective daily dose of the active compound may be administered as one, two, three, four, five, six or more sub-doses administered separately at appropriate intervals throughout the day, optionally, in unit dosage forms. In certain embodiments described herein, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds described herein may be used alone or conjointly administered with another type of therapeutic agent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations.

All publications and patents mentioned herein are hereby incorporated by reference in their entirety as if each individual publication or patent was specifically and individually indicated to be incorporated by reference. In case of conflict, the present application, including any definitions herein, will control.

### EXAMPLES

### Example 1: (E)-3-(1-(3,5-bis(trifluoromethyl)benzyl)-1H-pyrrolo[2,3-b]pyridin-3-yl)-2-cyanoacrylamide (PP1)

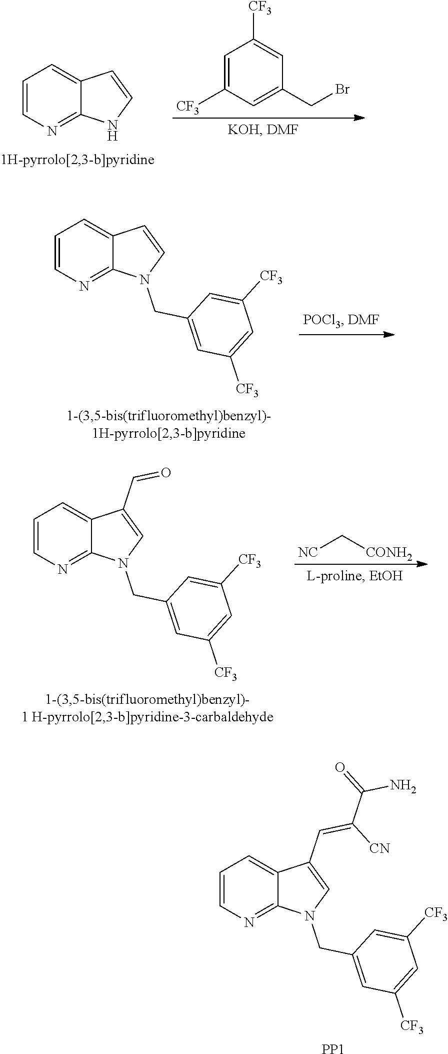

To the solution of 1H-pyrrolo[2,3-b]pyridine (1 equiv, 3 mmol, 354.4 mg) in dry DMF (6 mL) were added 3,5-bis(trifluoromethyl)benzyl bromide (1.2 equiv, 3.6 mmol, 660 μL) and KOH (1.2 equiv, 3.6 mmol, 201.9 mg) at 0 Sc. The reaction mixture was stirred at 21° C. for 2 h. After the reaction completion as shown by TLC, water (18 mL) was added to the reaction vial. The reaction mixture was extracted by dichloromethane (45 mL×3). The combined organic layer was dried by sodium sulfate and concentrated. The residue was purified by flash column chromatography (hexanes/EtOAc=12:1) to provide the desired product, 1-(3,5-bis(trifluoromethyl)benzyl)-1H-pyrrolo[2,3-b]pyridine (yield: 75%, 774.5 mg).

POCl₃ (1 equiv, 1 mmol, 93 μL) was added dropwise to DMF (2 mL) at 0° C. under argon. After the mixture stirred for 10 min, a solution of 1-(3,5-bis(trifluoromethyl)benzyl)-1H-pyrrolo[2,3-b]pyridine (1 equiv, 1 mmol, 344 mg) in DMF (2 mL) was added slowly with stirring. The mixture was kept at 21° C. overnight. The reaction was quenched by adding water (5 mL) at 0° C., then extracted with dichloromethane (10 mL×3). The combined organic layer was dried by sodium sulfate and concentrated. The residue was purified by flash column chromatography (hexanes/EtOAc=4:1) to provide the desired product, 1-(3,5-bis(trifluoromethyl)benzyl)-1H-pyrrolo[2,3-b]pyridine-3-carboxaldehyde (yield 89%, 332.9 mg).

To a solution of 1-(3,5-bis(trifluoromethyl)benzyl)-1H-pyrrolo[2,3-b]pyridine-3-carbox-aldehyde (1 equiv, 0.081 mmol, 30.0 mg) in ethanol (0.8 mL) was added 2-cyanoaceta-mide (1.3 equiv, 0.10 mmol, 8.8 mg) and L-proline (40 mol %, 0.0322 mmol, 3.7 mg). The reaction was stirred at 21° C. for 12 h and yellow solid precipitated gradually. After completion of the reaction, ice-cold water (0.8 mL) was added to the reaction vial. The solid was separated by Buchner funnel filtration and washed with water (0.8 mL×3) and dried to afford the desired product, PP1 (yield 91%, 32.2 mg).

¹H NMR (500 MHz, CDCl₃) δ 8.58 (d, J=0.7 Hz, 1H), 8.50 (s, 1H), 8.47 (dd, J=4.7, 1.5 Hz, 1H), 8.24 (dd, J=7.9, 1.5 Hz, 1H), 7.83 (s, 1H), 7.77 (s, 2H), 7.33 (dd, J=8.0, 4.7 Hz, 1H), 6.14 (br s, 1H), 5.69 (s, 2H), 5.61 (br s, 1H).

¹³C NMR (125 MHz, CDCl₃) δ 162.6, 147.6, 145.7, 144.2, 138.5, 132.5 (q, ²J_{C-F}=33.7 Hz, 2C), 132.0, 127.9, 127.9 (d, ³J_{C-F}=2.2 Hz), 122.9 (q, ¹J_{C-F}=272.9 Hz, 2C), 122.4 (p, ³J_{C-F}=2.2 Hz, 2C), 120.2, 119.0, 118.7, 109.5, 97.1, 48.2.

The following compounds were synthesized by a route similar to that described for PP1: PP2, PP3, PP4.

### Example 2: (E)-3-(1-(3,5-bis(trifluoromethyl)benzyl)-1H-pyrrolo[2,3-b]pyridin-3-yl)-2-cyano-N-methylacrylamide (PP2)

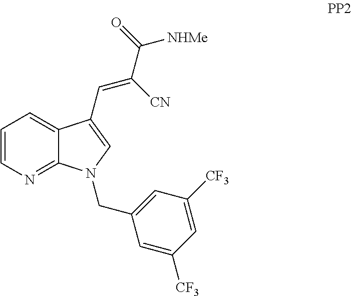 [SMILES: CNC(=O)/C(C#N)=C/c1cn(Cc2cc(C(F)(F)F)cc(C(F)(F)F)c2)c2ncccc12]

¹H NMR (500 MHz, CDCl₃) δ 8.56 (s, 1H), 8.46 (dd, J=4.6, 1.4 Hz, 1H), 8.45 (s, 1H), 8.24 (dd, J=7.9, 1.4 Hz, 1H), 7.82 (s, 1H), 7.76 (s, 2H), 7.32 (ddd, J=7.8, 4.7, 1.6 Hz, 1H), 6.20 (m, 1H), 5.68 (s, 2H), 3.01 (dd, J=4.9, 1.5 Hz, 3H).

¹³C NMR (125 MHz, CDCl₃) δ 161.6, 147.6, 145.5, 142.9, 138.6, 132.4 (q, ²J_{C-F}=33.7 Hz, 2C), 131.5, 128.0, 127.8 (d, ³J_{C-F}=2.6 Hz), 123.0 (q, ¹J_{C-F}=272.8 Hz, 2C), 122.4 (p, ³J_{C-F}=3.9 Hz, 2C), 120.2, 118.8, 118.7, 109.5, 98.3, 48.2, 27.2.

### Example 3: (E)-3-(1-(3,5-bis(trifluoromethyl)benzyl)-1H-pyrrolo[2,3-b]pyridin-3-yl)-2-cyano-N,N-dimethylacrylamide (PP3)

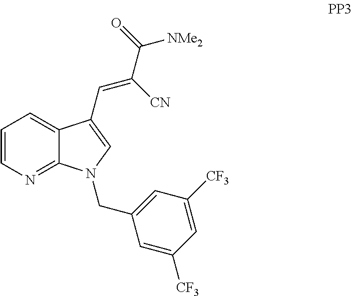 [SMILES: CN(C)C(=O)/C(C#N)=C/c1cn(Cc2cc(C(F)(F)F)cc(C(F)(F)F)c2)c2ncccc12]

¹H NMR (500 MHz, CDCl₃) δ 8.52 (s, 1H), 8.45 (dd, J=4.7, 1.5 Hz, 1H), 8.22 (d, J=0.3 Hz, 1H), 8.14 (dd, J=8.0, 1.5 Hz, 1H), 7.82 (s, 1H), 7.75 (s, 2H), 7.30 (dd, J=8.0, 4.7 Hz, 1H), 5.68 (s, 2H), 3.25 (br s, 3H), 3.10 (br s, 3H).

¹³C NMR (125 MHz, CDCl₃) δ 164.3, 147.4, 145.4, 143.8, 138.8, 132.3 (q, ²J_{C-F}=33.6 Hz, 2C), 130.9, 127.8 (d, ³J_{C-F}=3.4 Hz), 127.6, 123.0 (q, ¹J_{C-F}=273.0 Hz, 2C), 122.3 (p, ³J_{C-F}=3.7 Hz, 2C), 120.2, 118.6, 118.1, 109.6, 100.1, 48.1, 39.3, 36.9.

### Example 4: (E)-3-(1-(3,5-bis(trifluoromethyl)benzyl)-1H-pyrrolo[2,3-b]pyridin-3-yl)-2-(morpholine-4-carbonyl)acrylonitrile (PP4)

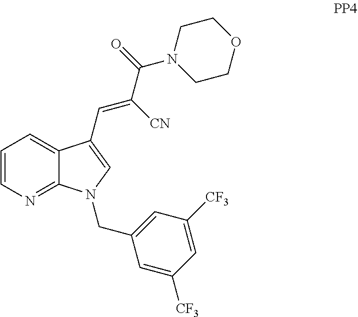 [SMILES: N#C/C(=C\c1cn(Cc2cc(C(F)(F)F)cc(C(F)(F)F)c2)c2ncccc12)C(=O)N1CCOCC1]

¹H NMR (500 MHz, CDCl₃) δ 8.51 (s, 1H), 8.46 (dd, J=4.7, 1.5 Hz, 1H), 8.23 (s, 1H), 8.15 (dd, J=7.9, 1.5 Hz, 1H), 7.82 (s, 1H), 7.75 (s, 2H), 7.31 (dd, J=8.0, 4.7 Hz, 1H), 5.68 (s, 2H), 3.75-3.77 (m, 8H).

¹³C NMR (125 MHz, CDCl₃) δ 163.6, 147.4, 145.5, 144.5, 138.7, 132.4 (q, ²J_{C-F}=33.6 Hz, 2C), 131.2, 127.8 (d, ³J_{C-F}=2.5 Hz), 127.6, 123.0 (q, ¹J_{C-F}=273.0 Hz, 2C), 122.3 (p, ³J_{C-F}=3.8 Hz, 2C), 120.1, 118.7, 118.2, 109.6, 99.2, 66.7 (4C), 48.1.

### Example 5: (E)-3-(1-(3,5-bis(trifluoromethyl)benzyl)-4-fluoro-1H-pyrrolo[2,3-b]pyridin-3-yl)-2-cyanoacrylic acid (PP8)

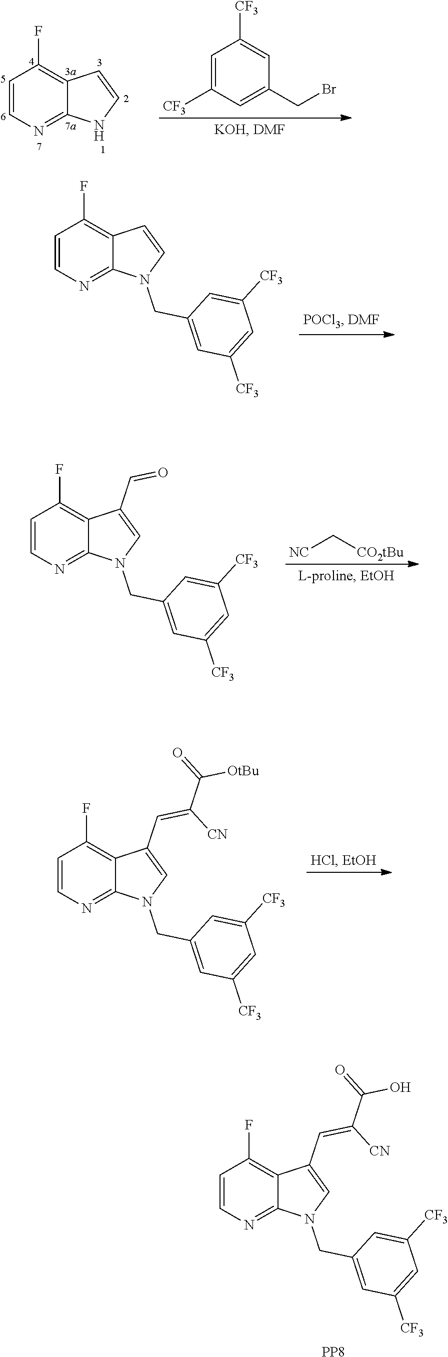

To the solution of 4-fluoro-1H-pyrrolo[2,3-b]pyridine (1.0 equiv, 4 mmol, 544.5 mg) in dry DMF (8 mL) were added 3,5-bis(trifluoromethyl)benzyl bromide (1.2 equiv, 4.8 mmol, 880 μL) and KOH (1.2 equiv, 4.8 mmol, 269.3 mg) at 0 Sc. The reaction mixture was stirred at 21° C. for 2 h. After the reaction completion as shown by TLC, water (24 mL) was added to the reaction vial. The reaction mixture was extracted by dichloromethane (60 mL×3). The combined organic layer was dried over sodium sulfate and concentrated. The residue was purified by flash column chromatography (hexanes/EtOAc=12:1) to provide the desired product, 1-(3,5-bis(trifluoromethyl)benzyl)-4-fluoro-1H-pyrrolo[2,3-b]pyridine (yield: 78%, 1132.0 mg).

POCl₃ (1.0 equiv, 1.38 mmol, 128.6 μL) was added dropwise to DMF (2.8 mL) at 0° C. under argon. After the mixture stirred for 10 min, a solution of 1-(3,5-bis(trifluoromethyl)benzyl)-4-fluoro-1H-pyrrolo[2,3-b]pyridine (1.0 equiv, 1.38 mmol, 500 mg) in DMF (2.8 mL) was added slowly with stirring. The mixture was kept at 21° C. overnight. The reaction was quenched by adding water (6.9 mL) at 0° C., then extracted with dichloromethane (13.8 mL×3). The combined organic layer was dried over sodium sulfate and concentrated. The residue was purified by flash column chromatography (hexanes/EtOAc=4:1) to provide the desired product, 1-(3,5-bis(trifluoromethyl)benzyl)-4-fluoro-1H-pyrrolo[2,3-b]pyridine-3-carboxaldehyde (yield 60%, 325.4 mg).

To the solution of 1-(3,5-bis(trifluoromethyl)benzyl)-4-fluoro-1H-pyrrolo[2,3-b]pyridine-3-carboxaldehyde (1.0 equiv, 0.256 mmol, 100.0 mg) in ethanol (1.0 mL) was added tert-butyl 2-cyanoacetate (1.3 equiv, 0.333 mmol, 41.9 μL) and L-proline (40 mol %, 0.1 mmol, 23.0 mg). The reaction was stirred at 21° C. for 12 h and yellow solid precipitated gradually. After completion of the reaction, ice-cold water (1.0 mL) was added to the reaction vial. The solid was separated by Buchner funnel filtration and washed with water (1.0 mL×3) and the solvent was evaporated in vacuo. The powder was dissolved in chloroform and subjected to column chromatography (hexanes/EtOAc=5:1) to provide the desired product, tert-butyl (E)-3-(1-(3,5-bis(trifluoromethyl)benzyl)-4-fluoro-1H-pyrrolo[2,3-b]pyridin-3-yl)-2-cyanoacrylate (yield 91%, 120.1 mg).

To a solution of tert-butyl (E)-3-(1-(3,5-bis(trifluoromethyl)benzyl)-4-fluoro-1H-pyrrolo[2,3-b]pyridin-3-yl)-2-cyanoacrylate (1.0 equiv, 0.1 mmol, 51.3 mg) in EtOH (1 mL) was added 12.0 M aq. HCl (120 equiv, 12.0 mmol, 1.0 mL). The reaction mixture was stirred at 21° C. for 12 h. After the reaction was complete as shown by TLC, the reaction solvent was evaporated in vacuo. The solid was washed by 1 mL of solvent mixture (hexanes/EtOAc=5:1) 5 to 10 times and monitored by TLC until all the non-polar impurities disappeared. Finally, the product was dried in vacuo yielding the desired product, PP8 (yield 78%, 35.5 mg).

¹H NMR (500 MHz, DMSO-d₆) δ 13.64 (br s, 1H), 8.91 (s, 1H), 8.42-8.45 (m, 2H), 8.13 (s, 2H), 8.05 (s, 1H), 7.28 (dd, J=10.8, 5.5 Hz, 1H), 5.86 (s, 2H).

¹³C NMR (125 MHz, DMSO-d₆) δ 164.2, 163.0 (d, ¹J_{C-F}=262.6 Hz), 150.7 (d, ³J_{C-F}=10.6 Hz), 147.6 (d, ³J_{C-F}=7.1 Hz), 146.0 (d, ³J_{C-F}=3.7 Hz), 140.3, 134.2, 130.9 (q, ²J_{C-F}=32.9 Hz, 2C), 129.5 (d, ³J_{C-F}=3.8 Hz), 123.6 (q, ¹J_{C-F}=272.8 Hz, 2C), 122.4 (p, ³J_{C-F}=3.7 Hz, 2C), 117.6, 108.9 (d, ²J_{C-F}=14.8 Hz), 106.7 (d, ⁴J_{C-F}=2.7 Hz), 106.3 (d, ²J_{C-F}=15.4 Hz), 98.4, 48.3.

The following compounds were synthesized by a route similar to that described for PP8: PP5, PP6, PP7, PP9, PP10, PP11, PP12, PP13, PP14, PP15, PP16, PP17, PP18, PP19, PP20, PP21, PP22, PP23, PP24, PP25, PP26, PP27, PP28, PP29, and PP30.

### Example 6: Methyl (E)-3-(1-(3,5-bis(trifluoromethyl)benzyl)-1H-pyrrolo[2,3-b]pyridin-3-yl)-2-cyanoacrylate (PP5)

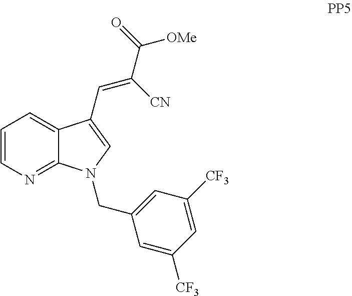 [SMILES: COC(=O)/C(C#N)=C/c1cn(Cc2cc(C(F)(F)F)cc(C(F)(F)F)c2)c2ncccc12]

¹H NMR (500 MHz, CDCl₃) δ 8.64 (s, 1H), 8.51 (d, J=0.5 Hz, 1H), 8.47 (dd, J=4.7, 1.4 Hz, 1H), 8.21 (dd, J=8.0, 1.5 Hz, 1H), 7.82 (s, 1H), 7.77 (s, 2H), 7.34 (dd, J=8.0, 4.7 Hz, 1H), 5.68 (s, 2H), 3.92 (s, 3H).

¹³C NMR (125 MHz, CDCl₃) δ 163.7, 147.6, 145.7, 145.2, 138.4, 132.9, 132.4 (q, ²J_{C-F}=33.7 Hz, 2C), 127.9 (d, ³J_{C-F}=3.8 Hz), 127.7, 123.0 (q, ¹J_{C-F}=272.9 Hz, 2C), 122.4 (p, ³J_{C-F}=3.9 Hz, 2C), 120.3, 119.1, 117.5, 109.3, 96.5, 53.1, 48.3.

### Example 7: Ethyl (E)-3-(1-(3,5-bis(trifluoromethyl)benzyl)-4-fluoro-1H-pyrrolo[2,3-b]pyridin-3-yl)-2-cyanoacrylate (PP6)

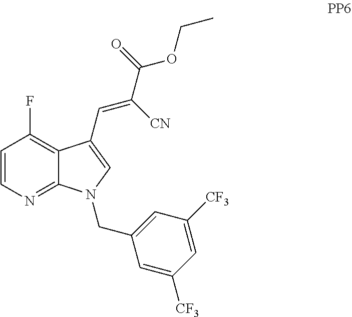 [SMILES: CCOC(=O)/C(C#N)=C/c1cn(Cc2cc(C(F)(F)F)cc(C(F)(F)F)c2)c2nccc(F)c12]

¹H NMR (500 MHz, CDCl₃) δ 8.68 (s, 1H), 8.67 (s, 1H), 8.39 (dd, J=7.4, 5.5 Hz, 1H), 7.83 (s, 1H), 7.77 (s, 2H), 7.03 (dd, J=10.1, 5.4 Hz, 1H), 5.67 (s, 2H), 4.37 (q, J=7.1 Hz, 2H), 1.39 (t, J=7.1 Hz, 3H).

¹³C NMR (125 MHz, CDCl₃) δ 163.3 (d, ¹J_{C-F}=266.0 Hz), 162.8, 150.4 (d, ³J_{C-F}=10.6 Hz), 147.3 (d, ³J_{C-F}=6.9 Hz), 146.3 (d, ³J_{C-F}=4.1 Hz), 138.1, 132.5 (q, ²J_{C-F}=33.6 Hz, 2C), 132.2 (d, ⁴J_{C-F}=0.9 Hz), 127.9 (d, ³J_{C-F}=3.8 Hz), 122.9 (q, ¹J_{C-F}=273.0 Hz, 2C), 122.5 (p, ³J_{C-F}=3.8 Hz, 2C), 117.4, 109.2 (d, ²J_{C-F}=15.1 Hz), 108.1 (d, ⁴J_{C-F}=2.9 Hz), 106.1 (d, ²J_{C-F}=15.5 Hz), 98.3, 62.4, 48.7, 14.2.

### Example 8: Methyl (E)-3-(1-(3,5-bis(trifluoromethyl)benzyl)-4-fluoro-1H-pyrrolo[2,3-b]pyridin-3-yl)-2-cyanoacrylate (PP7)

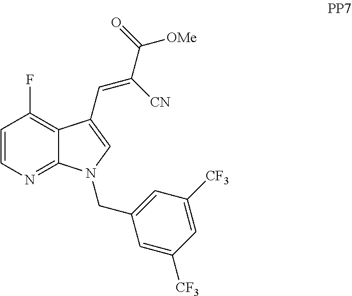 [SMILES: COC(=O)/C(C#N)=C/c1cn(Cc2cc(C(F)(F)F)cc(C(F)(F)F)c2)c2nccc(F)c12]

¹H NMR (500 MHz, CDCl₃) δ 8.68 (s, 1H), 8.67 (s, 1H), 8.40 (dd, J=7.4, 5.4 Hz, 1H), 7.83 (s, 1H), 7.77 (s, 2H), 7.04 (dd, J=10.1, 5.4 Hz, 1H), 5.67 (s, 2H), 3.92 (s, 3H).

¹³C NMR (125 MHz, CDCl₃) δ 163.3 (d, ¹J_{C-F}=266.0 Hz), 163.3, 150.4 (d, ³J_{C-F}=10.7 Hz), 147.3 (d, ³J_{C-F}=7.0 Hz), 146.6 (d, ³J_{C-F}=4.0 Hz), 138.1, 132.5 (q, ²J_{C-F}=33.8 Hz, 2C), 132.3 (d, ⁴J_{C-F}=1.6 Hz), 127.9 (d, ³J_{C-F}=3.8 Hz), 122.9 (q, ¹J_{C-F}=272.9 Hz, 2C), 122.6 (p, ³J_{C-F}=3.8 Hz, 2C), 117.4, 109.2 (d, ²J_{C-F}=15.2 Hz), 108.1 (d, ⁴J_{C-F}=2.8 Hz), 106.1 (d, ²J_{C-F}=15.6 Hz), 97.8, 53.1, 48.7.

### Example 9: Propyl (E)-3-(1-(3,5-bis(trifluoromethyl)benzyl)-1H-pyrrolo[2,3-b]pyridin-3-yl)-2-cyanoacrylate (PP9)

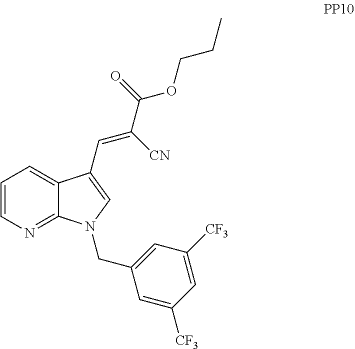 [SMILES: CCCOC(=O)/C(C#N)=C/c1cn(Cc2cc(C(F)(F)F)cc(C(F)(F)F)c2)c2ncccc12]

¹H NMR (500 MHz, CDCl₃) δ 8.64 (s, 1H), 8.50 (d, J=0.7 Hz, 1H), 8.47 (dd, J=4.7, 1.5 Hz, 1H), 8.22 (dd, J=8.0, 1.5 Hz, 1H), 7.82 (s, 1H), 7.77 (s, 2H), 7.34 (dd, J=8.0, 4.7 Hz, 1H), 5.71 (s, 2H), 4.27 (t, J=6.7 Hz, 2H), 1.79 (heptet, J=7.3 Hz, 2H), 1.02 (t, J=7.4 Hz, 3H).

¹³C NMR (125 MHz, CDCl₃) δ 163.4, 147.5, 145.6, 145.0, 138.6, 132.9, 132.5 (q, ²J_{C-F}=33.7 Hz, 2C), 128.0 (2C), 123.1 (q, ¹J_{C-F}=272.9 Hz, 2C), 122.6 (p, ³J_{C-F}=3.7 Hz, 2C), 120.6, 119.1, 117.6, 109.5, 97.4, 67.9, 48.5, 22.2, 10.5.

### Example 10: Isopropyl (E)-3-(1-(3,5-bis(trifluoromethyl)benzyl)-1H-pyrrolo[2,3-b]pyridin-3-yl)-2-cyanoacrylate (PP10)

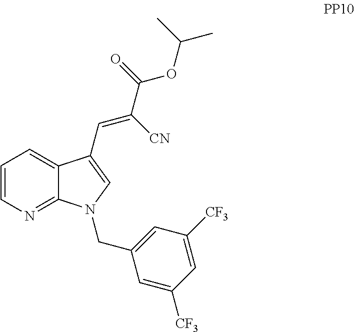 [SMILES: CC(C)OC(=O)/C(C#N)=C/c1cn(Cc2cc(C(F)(F)F)cc(C(F)(F)F)c2)c2ncccc12]

¹H NMR (500 MHz, CDCl₃) δ 8.63 (s, 1H), 8.48 (s, 1H), 8.47 (dd, J=4.7, 1.5 Hz, 1H), 8.21 (dd, J=7.9, 1.5 Hz, 1H), 7.82 (s, 1H), 7.76 (s, 2H), 7.33 (dd, J=8.0, 4.7 Hz, 1H), 5.70 (s, 2H), 5.20 (hept, J=6.3 Hz, 1H), 1.37 (d, J=6.3 Hz, 6H).

¹³C NMR (125 MHz, CDCl₃) δ 162.7, 147.4, 145.5, 144.7, 138.5, 132.6, 132.4 (q, ²J_{C-F}=33.7 Hz, 2C), 127.8 (d, ³J_{C-F}=4.1 Hz), 127.8, 122.9 (q, ¹J_{C-F}=272.9 Hz, 2C), 122.4 (p, ³J_{C-F}=3.9 Hz, 2C), 120.4, 118.9, 117.5, 109.4, 97.7, 70.2, 48.3, 21.8 (2C).

### Example 11: Butyl (E)-3-(1-(3,5-bis(trifluoromethyl)benzyl)-1H-pyrrolo[2,3-b]pyridin-3-yl)-2-cyanoacrylate (PP11)

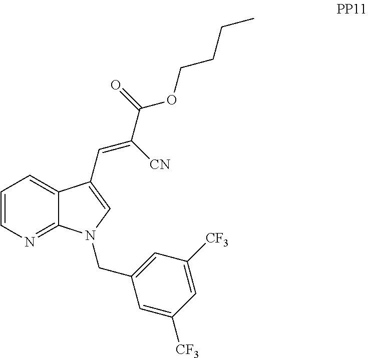 [SMILES: CCCCOC(=O)/C(C#N)=C/c1cn(Cc2cc(C(F)(F)F)cc(C(F)(F)F)c2)c2ncccc12]

¹H NMR (500 MHz, CDCl₃) δ 8.64 (s, 1H), 8.50 (s, 1H), 8.47 (dd, J=4.7, 1.5 Hz, 1H), 8.22 (dd, J=7.9, 1.5 Hz, 1H), 7.82 (s, 1H), 7.77 (s, 2H), 7.34 (dd, J=8.0, 4.7 Hz, 1H), 5.70 (s, 2H), 4.32 (t, J=6.7 Hz, 2H), 1.72-1.77 (m, 2H), 1.47 (heptet, J=7.4 Hz, 2H), 0.98 (t, J=7.4 Hz, 3H).

¹³C NMR (125 MHz, CDCl₃) δ 163.3, 147.4, 145.5, 144.8, 138.4, 132.7, 132.4 (q, ²J_{C-F}=33.6 Hz, 2C), 127.9, 127.8, 122.9 (q, ¹J_{C-F}=273.0 Hz, 2C), 122.4 (p, ³J_{C-F}=3.9 Hz, 2C), 120.4, 118.9, 117.5, 109.4, 97.2, 66.1, 48.4, 30.6, 19.1, 13.7.

### Example 12: Isobutyl (E)-3-(1-(3,5-bis(trifluoromethyl)benzyl)-1H-pyrrolo[2,3-b]pyridin-3-yl)-2-cyanoacrylate (PP12)

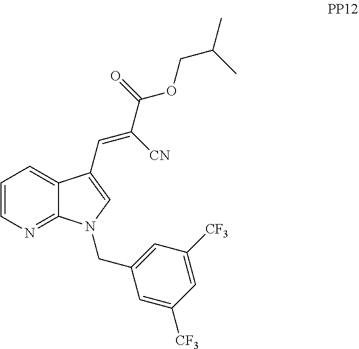 [SMILES: CC(C)COC(=O)/C(C#N)=C/c1cn(Cc2cc(C(F)(F)F)cc(C(F)(F)F)c2)c2ncccc12]

¹H NMR (500 MHz, CDCl₃) δ 8.64 (s, 1H), 8.50 (s, 1H), 8.47 (dd, J=4.7, 1.5 Hz, 1H), 8.22 (dd, J=8.0, 1.5 Hz, 1H), 7.82 (s, 1H), 7.77 (s, 2H), 7.34 (dd, J=8.0, 4.7 Hz, 1H), 5.70 (s, 2H), 4.09 (d, J=6.7 Hz, 2H), 2.08 (nonet, J=6.7 Hz, 1H), 1.02 (d, J=6.7 Hz, 6H).

¹³C NMR (125 MHz, CDCl₃) δ 163.3, 147.4, 145.5, 144.8, 138.4, 132.7, 132.4 (q, ²J_{C-F}=33.5 Hz, 2C), 127.9 (d, ³J_{C-F}=3.9 Hz), 127.8, 122.9 (q, ¹J_{C-F}=273.0 Hz, 2C), 122.4 (p, ³J_{C-F}=3.9 Hz, 2C), 120.4, 118.9, 117.4, 109.4, 97.2, 72.2, 48.4, 27.8, 19.0 (2C).

### Example 13: 2-Methoxyethyl (E)-3-(1-(3,5-bis(trifluoromethyl)benzyl)-1H-pyrrolo[2,3-b]pyridin-3-yl)-2-cyanoacrylate (PP13)

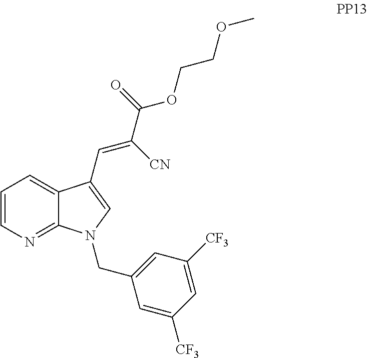 [SMILES: COCCOC(=O)/C(C#N)=C/c1cn(Cc2cc(C(F)(F)F)cc(C(F)(F)F)c2)c2ncccc12]

¹H NMR (500 MHz, CDCl₃) δ 8.65 (s, 1H), 8.51 (d, J=0.7 Hz, 1H), 8.47 (dd, J=4.7, 1.4 Hz, 1H), 8.22 (dd, J=8.0, 1.5 Hz, 1H), 7.82 (s, 1H), 7.77 (s, 2H), 7.34 (dd, J=8.0, 4.7 Hz, 1H), 5.70 (s, 2H), 4.45-4.47 (m, 2H), 3.70-3.72 (m, 2H), 3.43 (s, 3H).

¹³C NMR (125 MHz, CDCl₃) δ 163.2, 147.4, 145.5, 145.2, 138.4, 132.9, 132.4 (q, ²J_{C-F}=33.8 Hz, 2C), 127.9, 127.8, 122.9 (q, ¹J_{C-F}=273.0 Hz, 2C), 122.4 (p, ³J_{C-F}=3.9 Hz, 2C), 120.4, 119.0, 117.4, 109.4, 96.8, 70.2, 65.2, 59.2, 48.4.

### Example 14: 2-Ethoxyethyl (E)-3-(1-(3,5-bis(trifluoromethyl)benzyl)-1H-pyrrolo[2,3-b]pyridin-3-yl)-2-cyanoacrylate (PP14)

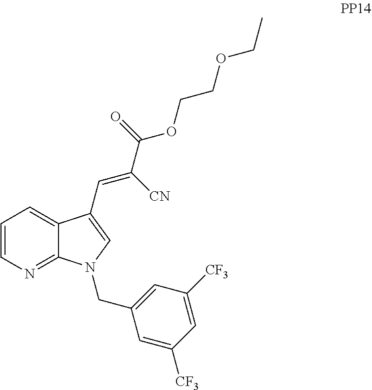 [SMILES: CCOCCOC(=O)/C(C#N)=C/c1cn(Cc2cc(C(F)(F)F)cc(C(F)(F)F)c2)c2ncccc12]

¹H NMR (500 MHz, CDCl₃) δ 8.65 (s, 1H), 8.51 (d, J=0.6 Hz, 1H), 8.48 (dd, J=4.7, 1.4 Hz, 1H), 8.22 (dd, J=8.0, 1.5 Hz, 1H), 7.82 (s, 1H), 7.77 (s, 2H), 7.35 (dd, J=8.0, 4.7 Hz, 1H), 5.71 (s, 2H), 4.44-4.46 (m, 2H), 3.74-3.76 (m, 2H), 3.59 (q, J=7.0 Hz, 2H), 1.23 (t, J=7.0 Hz, 3H).

¹³C NMR (125 MHz, CDCl₃) δ 163.2, 147.3, 145.4, 145.1, 138.4, 132.9, 132.4 (q, ²J_{C-F}=33.7 Hz, 2C), 127.9, 127.9 (d, ³J_{C-F}=4.3 Hz), 122.9 (q, ¹J_{C-F}=272.8 Hz, 2C), 122.5 (p, ³J_{C-F}=3.6 Hz, 2C), 120.4, 119.0, 117.4, 109.4, 97.0, 68.0, 66.8, 65.4, 48.4, 15.2.

### Example 15: Octyl (E)-3-(1-(3,5-bis(trifluoromethyl)benzyl)-1H-pyrrolo[2,3-b]pyridin-3-yl)-2-cyanoacrylate (PP15)

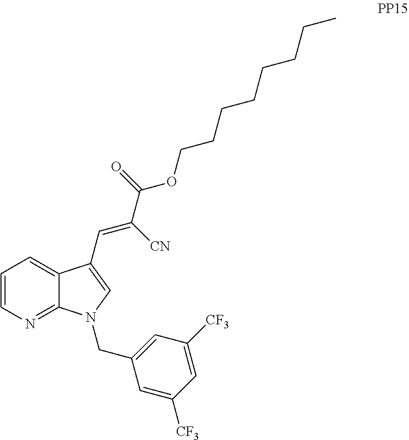 [SMILES: CCCCCCCCOC(=O)/C(C#N)=C/c1cn(Cc2cc(C(F)(F)F)cc(C(F)(F)F)c2)c2ncccc12]

¹H NMR (500 MHz, CDCl₃) δ 8.64 (s, 1H), 8.50 (d, J=0.7 Hz, 1H), 8.47 (dd, J=4.7, 1.5 Hz, 1H), 8.20 (dd, J=7.9, 1.5 Hz, 1H), 7.82 (s, 1H), 7.76 (s, 2H), 7.33 (dd, J=8.0, 4.7 Hz, 1H), 5.69 (s, 2H), 4.30 (t, J=6.8 Hz, 2H), 1.72-1.78 (m, 2H), 1.28-1.45 (m, 10H), 0.88 (t, J 2=6.9 Hz, 3H).

¹³C NMR (125 MHz, CDCl₃) δ 163.3, 147.6, 145.7, 144.9, 138.5, 132.7, 132.4 (q, ²J_{C-F}=33.7 Hz, 2C), 127.8 (d, ³J_{C-F}=3.8 Hz), 127.6, 122.9 (q, ¹J_{C-F}=272.8 Hz, 2C), 122.4 (p, ³J_{C-F}=3.7 Hz, 2C), 120.3, 119.0, 117.5, 109.3, 97.1, 66.4, 48.3, 31.8, 29.2, 29.2, 28.6, 25.8, 22.7, 14.1.

### Example 16: Cyclohexyl (E)-3-(1-(3,5-bis(trifluoromethyl)benzyl)-1H-pyrrolo[2,3-b]pyridin-3-yl)-2-cyanoacrylate (PP16)

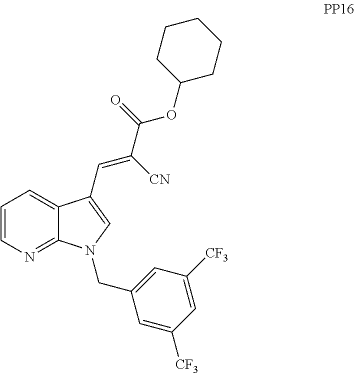 [SMILES: N#C/C(=C\c1cn(Cc2cc(C(F)(F)F)cc(C(F)(F)F)c2)c2ncccc12)C(=O)OC1CCCCC1]

¹H NMR (500 MHz, CDCl₃) δ 8.63 (s, 1H), 8.48 (s, 1H), 8.47 (dd, J=4.7, 1.4 Hz, 1H), 8.22 (dd, J=8.0, 1.5 Hz, 1H), 7.82 (s, 1H), 7.76 (s, 2H), 7.33 (dd, J=8.0, 4.7 Hz, 1H), 5.70 (s, 2H), 4.98 (ddd, J=12.7, 8.9, 3.8 Hz, 1H), 1.90-1.93 (m, 2H), 1.78-1.83 (m, 2H), 1.31-1.64 (m, 6H).

¹³C NMR (125 MHz, CDCl₃) δ 162.6, 147.5, 145.6, 144.6, 138.5, 132.6, 132.4 (q, ²J_{C-F}=33.5 Hz, 2C), 127.8 (d, ³J_{C-F}=3.7 Hz), 127.7, 123.0 (q, ¹J_{C-F}=272.8 Hz, 2C), 122.4 (p, ³J_{C-F}=3.9 Hz, 2C), 120.3, 118.9, 117.6, 109.3, 97.7, 74.8, 48.3, 31.5 (2C), 25.3 (2C), 23.5.

### Example 17: 1-(3,5-bis(trifluoromethyl)benzyl)-1H-pyrrolo[2,3-b]pyridine-3-carboxaldehyde (PP17)

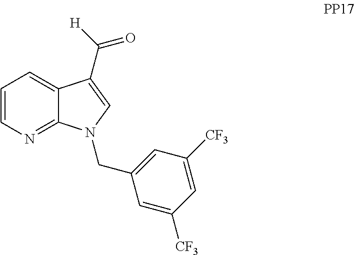 [SMILES: O=Cc1cn(Cc2cc(C(F)(F)F)cc(C(F)(F)F)c2)c2ncccc12]

¹H NMR (500 MHz, CDCl₃) δ 10.00 (s, 1H), 8.60 (dd, J=7.8, 1.6 Hz, 1H), 8.46 (dd, J=4.8, 1.6 Hz, 1H), 7.87 (s, 1H), 7.84 (s, 1H), 7.77 (s, 2H), 7.32 (dd, J=7.9, 4.8 Hz, 1H), 5.66 (s, 2H).

¹³C NMR (125 MHz, CDCl₃) δ 184.6, 148.3, 145.7, 138.7, 136.9, 132.5 (q, ²J_{C-F}=33.6 Hz, 2C), 131.0, 128.0, 127.9, 122.9 (q, ¹J_{C-F}=272.8 Hz, 2C), 122.4 (p, ³J_{C-F}=3.9 Hz, 2C), 119.5, 117.6, 47.9.

### Example 18: Phenyl (E)-3-(1-(3,5-bis(trifluoromethyl)benzyl)-1H-pyrrolo[2,3-b]pyridine-3-yl)-2-cyanoacrylate (PP18)

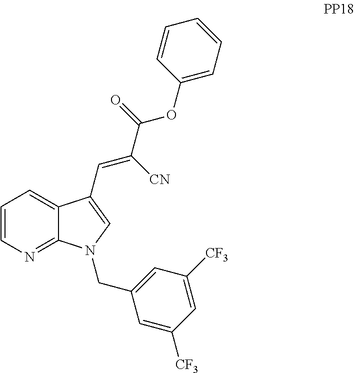 [SMILES: N#C/C(=C\c1cn(Cc2cc(C(F)(F)F)cc(C(F)(F)F)c2)c2ncccc12)C(=O)Oc1ccccc1]

¹H NMR (500 MHz, CDCl₃) δ 8.74 (s, 1H), 8.64 (s, 1H), 8.49 (dd, J=4.6, 1.5 Hz, 1H), 8.24 (dd, J=7.9, 1.5 Hz, 1H), 7.82 (s, 1H), 7.79 (s, 2H), 7.41-7.45 (m, 2H), 7.36 (dd, J=7.9, 4.7 Hz, 1H), 7.29 (t, J=7.5 Hz, 1H), 7.21-7.23 (m, 2H), 5.72 (s, 2H).

¹³C NMR (125 MHz, CDCl₃) δ 162.1, 150.5, 147.7, 146.4, 145.9, 138.3, 133.4, 132.4 (q, ²J_{C-F}=33.7 Hz, 2C), 129.6 (2C), 127.9 (d, ³J_{C-F}=3.8 Hz), 127.7, 126.3, 122.9 (q, ¹J_{C-F}=272.9 Hz, 2C), 122.5 (p, ³J_{C-F}=3.8 Hz, 2C), 121.4 (2C), 120.3, 119.2, 117.4, 109.5, 96.0, 48.4.

### Example 19: 3,5-Bis(trifluoromethyl)benzyl (E)-3-(1-(3,5-bis(trifluoromethyl)benzyl)-1H-pyrrolo[2,3-b]pyridin-3-yl)-2-cyanoacrylate (PP19)

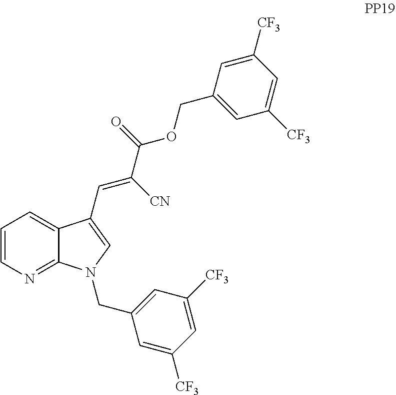 [SMILES: N#C/C(=C\c1cn(Cc2cc(C(F)(F)F)cc(C(F)(F)F)c2)c2ncccc12)C(=O)OCc1cc(C(F)(F)F)cc(C(F)(F)F)c1]

¹H NMR (500 MHz, CDCl₃) δ 8.68 (s, 1H), 8.54 (s, 1H), 8.48 (dd, J=4.7, 1.5 Hz, 1H), 8.21 (dd, J=8.0, 1.5 Hz, 1H), 7.91 (s, 2H), 7.88 (s, 1H), 7.83 (s, 1H), 7.77 (s, 2H), 7.35 (dd, J=8.0, 4.7 Hz, 1H), 5.69 (s, 2H), 5.44 (s, 2H).

¹³C NMR (125 MHz, CDCl₃) δ 163.0, 147.6, 146.1, 145.9, 138.3, 137.8, 133.4, 132.4 (q, ²J_{C-F}=33.3 Hz, 2C), 132.3 (q, ²J_{C-F}=33.2 Hz, 2C), 128.3 (d, ³J_{C-F}=3.8 Hz, 2C), 127.9 (d, ³J_{C-F}=3.8 Hz, 2C), 127.7, 123.1 (q, ¹J_{C-F}=272.7 Hz, 2C), 122.9 (q, ¹J_{C-F}=272.3 Hz, 2C), 122.5 (m, 2C), 120.2, 119.2, 117.2, 109.4, 95.6, 65.8, 48.4.

### Example 20: Acetyloxymethyl (E)-3-(1-(3,5-bis(trifluoromethyl)benzyl)-1H-pyrrolo[2,3-b]pyridin-3-yl)-2-cyanoacrylate (PP20)

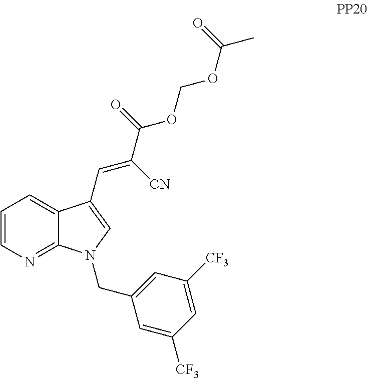 [SMILES: CC(=O)OCOC(=O)/C(C#N)=C/c1cn(Cc2cc(C(F)(F)F)cc(C(F)(F)F)c2)c2ncccc12]

¹H NMR (500 MHz, CDCl₃) δ 8.69 (s, 1H), 8.54 (d, J=0.7 Hz, 1H), 8.48 (dd, J=4.7, 1.5 Hz, 1H), 8.21 (dd, J=8.0, 1.5 Hz, 1H), 7.83 (s, 1H), 7.77 (s, 2H), 7.35 (dd, J=8.0, 4.7 Hz, 1H), 5.94 (s, 2H), 5.69 (s, 2H), 2.15 (s, 3H).

¹³C NMR (125 MHz, CDCl₃) δ 169.4, 162.2, 147.6, 146.3, 145.9, 138.3, 133.6, 132.4 (q, ²J_{C-F}=33.5 Hz, 2C), 127.9 (d, ³J_{C-F}=3.4 Hz), 127.7, 122.9 (q, ¹J_{C-F}=272.9 Hz, 2C), 122.5 (p, ³J_{C-F}=3.8 Hz, 2C), 120.3, 119.2, 117.0, 109.4, 95.5, 80.1, 48.4, 20.7.

### Example 21: Oxetan-3-yl (E)-3-(1-(3,5-bis(trifluoromethyl)benzyl)-1H-pyrrolo[2,3-b]pyridin-3-yl)-2-cyanoacrylate (PP21)

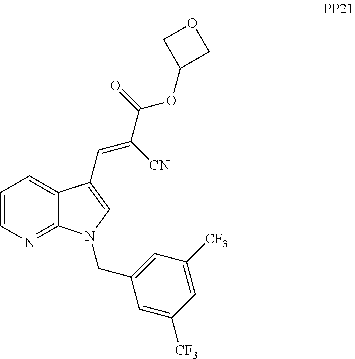 [SMILES: N#C/C(=C\c1cn(Cc2cc(C(F)(F)F)cc(C(F)(F)F)c2)c2ncccc12)C(=O)OC1COC1]

¹H NMR (500 MHz, CDCl₃) δ 8.68 (s, 1H), 8.51 (s, 1H), 8.49 (dd, J=4.7, 1.5 Hz, 1H), 8.21 (dd, J=8.0, 1.5 Hz, 1H), 7.83 (s, 1H), 7.78 (s, 2H), 7.35 (dd, J=7.9, 4.7 Hz, 1H), 5.70 (s, 2H), 5.63 (pentet, J=5.9 Hz, 1H), 4.96 (dd, J=7.2 Hz, 2H), 4.79 (dd, J=8.0, 5.4 Hz, 2H).

¹³C NMR (125 MHz, CDCl₃) δ 162.5, 147.6, 145.9 (2C), 138.3, 133.3, 132.4 (q, ²J_{C-F}=33.8 Hz, 2C), 127.9 (d, ³J_{C-F}=3.8 Hz), 127.7, 122.9 (q, ¹J_{C-F}=272.9 Hz, 2C), 122.5 (p, ³J_{C-F}=3.8 Hz, 2C), 120.3, 119.2, 117.2, 109.4, 95.6, 69.3 (3C), 48.4.

### Example 22: 1-Benzhydrylazetidin-3-yl (E)-3-(1-(3,5-bis(trifluoromethyl)benzyl)-1H-pyrrolo[2,3-b]pyridin-3-yl)-2-cyanoacrylate (PP22)

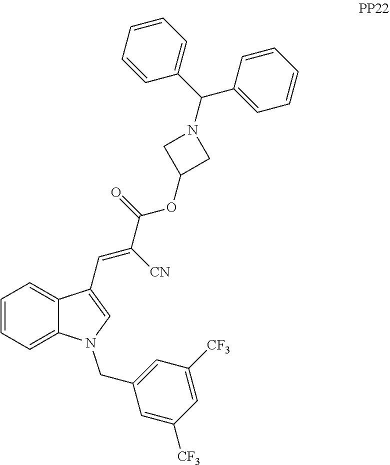 [SMILES: N#C/C(=C\c1cn(Cc2cc(C(F)(F)F)cc(C(F)(F)F)c2)c2ccccc12)C(=O)OC1CN(C(c2ccccc2)c2ccccc2)C1]

¹H NMR (500 MHz, CDCl₃) δ 8.65 (s, 1H), 8.48 (d, J=1.5 Hz, 1H), 8.47 (s, 1H), 8.19 (dd, J=8.0, 1.5 Hz, 1H), 7.83 (s, 1H), 7.77 (s, 2H), 7.41-7.42 (m, 4H), 7.33 (dd, J=8.0, 4.7 Hz, 1H), 7.28 (t, J=7.6 Hz, 4H), 7.19 (t, J=7.3 Hz, 2H), 5.69 (s, 2H), 5.24-5.29 (m, 1H), 4.43 (s, 1H), 3.69 (t, J=7.7 Hz, 2H), 3.16 (t, J=7.5 Hz, 2H).

¹³C NMR (125 MHz, CDCl₃) δ 162.6, 147.6, 145.8, 145.5, 141.7 (2C), 138.4, 133.0, 132.4 (q, ²J_{C-F}=33.6 Hz, 2C), 128.5 (4C), 127.9 (d, ³J_{C-F}=3.8 Hz), 127.7, 127.4 (4C), 127.3 (2C), 122.9 (q, ¹J_{C-F}=272.8 Hz, 2C), 122.5 (p, ³J_{C-F}=3.5 Hz, 2C), 120.3, 119.1, 117.4, 109.4, 96.2, 78.2, 65.3, 60.0 (2C), 48.4.

### Example 23: 3-Methoxypropyl (E)-3-(1-(3,5-bis(trifluoromethyl)benzyl)-1H-pyrrolo[2,3-b]pyridin-3-yl)-2-cyanoacrylate (PP23)

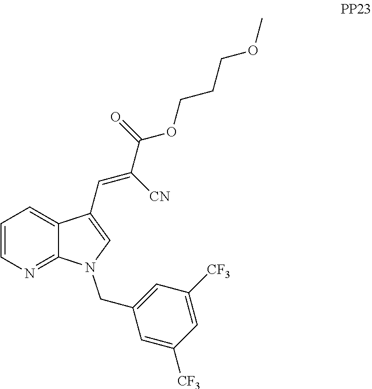 [SMILES: COCCCOC(=O)/C(C#N)=C/c1cn(Cc2cc(C(F)(F)F)cc(C(F)(F)F)c2)c2ncccc12]

¹H NMR (500 MHz, CDCl₃) δ 8.64 (s, 1H), 8.50 (s, 1H), 8.47 (dd, J=4.7, 1.5 Hz, 1H), 8.21 (dd, J=8.0, 1.5 Hz, 1H), 7.82 (s, 1H), 7.77 (s, 2H), 7.33 (dd, J=8.0, 4.7 Hz, 1H), 5.69 (s, 2H), 4.40 (t, J=6.4 Hz, 2H), 3.53 (t, J=6.1 Hz, 2H), 3.36 (s, 3H), 2.02 (pentet, J=6.3 Hz, 2H).

¹³C NMR (125 MHz, CDCl₃) δ 163.2, 147.6, 145.7, 145.1, 138.5, 132.8, 132.3 (q, ²J_{C-F}=33.4 Hz, 2C), 127.9 (d, ³J_{C-F}=3.0 Hz), 127.7, 122.9 (q, ¹J_{C-F}=272.9 Hz, 2C), 122.4 (p, ³J_{C-F}=3.8 Hz, 2C), 120.3, 119.0, 117.5, 109.3, 96.9, 68.8, 63.4, 58.8, 48.3, 28.9.

### Example 24: (Pivaloyloxy)methyl (E)-3-(1-(3,5-bis(trifluoromethyl)benzyl)-1H-pyrrolo[2,3-b]pyridin-3-yl)-2-cyanoacrylate (PP24)

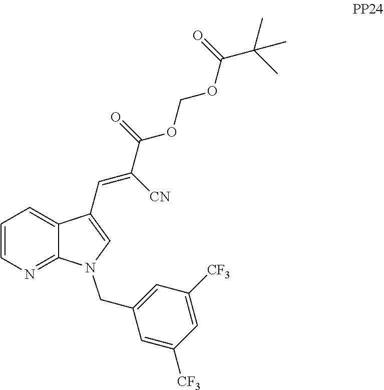 [SMILES: CC(C)(C)C(=O)OCOC(=O)/C(C#N)=C/c1cn(Cc2cc(C(F)(F)F)cc(C(F)(F)F)c2)c2ncccc12]

¹H NMR (500 MHz, CDCl₃) δ 8.68 (s, 1H), 8.54 (d, J=0.6 Hz, 1H), 8.48 (dd, J=4.7, 1.5 Hz, 1H), 8.21 (dd, J=8.0, 1.5 Hz, 1H), 7.82 (s, 1H), 7.77 (s, 2H), 7.35 (dd, J=8.0, 4.7 Hz, 1H), 5.98 (s, 2H), 5.69 (s, 2H), 1.23 (s, 9H).

¹³C NMR (125 MHz, CDCl₃) δ 176.9, 162.1, 147.6, 146.2, 145.9, 138.3, 133.5, 132.4 (q, ²J_{C-F}=33.7 Hz, 2C), 127.9 (d, ³J_{C-F}=3.5 Hz), 127.7, 122.9 (q, ¹J_{C-F}=272.9 Hz, 2C), 122.5 (p, ³J_{C-F}=3.8 Hz, 2C), 120.3, 119.2, 117.0, 109.4, 95.6, 80.5, 48.4, 38.8, 26.9 (3C).

### Example 25: (S)-1-Ethoxy-1-oxopropan-2-yl (E)-3-(1-(3,5-bis(trifluoromethyl)benzyl)-1H-pyrrolo[2,3-b]pyridin-3-yl)-2-cyanoacrylate (PP25)

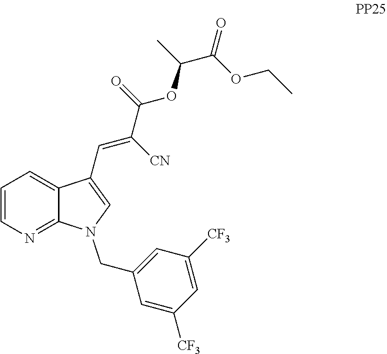 [SMILES: CCOC(=O)[C@H](C)OC(=O)/C(C#N)=C/c1cn(Cc2cc(C(F)(F)F)cc(C(F)(F)F)c2)c2ncccc12]

¹H NMR (500 MHz, CDCl₃) δ 8.68 (s, 1H), 8.53 (d, J=0.6 Hz, 1H), 8.47 (dd, J=4.7, 1.5 Hz, 1H), 8.19 (dd, J=8.0, 1.5 Hz, 1H), 7.82 (s, 1H), 7.77 (s, 2H), 7.33 (dd, J=8.0, 4.7 Hz, 1H), 5.69 (s, 2H), 5.24 (q, J=7.0 Hz, 1H), 4.24 (q, J=7.1 Hz, 2H), 1.63 (d, J=7.1 Hz, 3H), 1.29 (t, J=7.1 Hz, 3H).

¹³C NMR (125 MHz, CDCl₃) δ 170.1, 162.7, 147.6, 145.8 (2C), 138.4, 133.2, 132.4 (q, ²J_{C-F}=33.7 Hz, 2C), 127.9 (d, ³J_{C-F}=3.6 Hz), 127.7, 122.9 (q, ¹J_{C-F}=272.9 Hz, 2C), 122.4 (p, ³J_{C-F}=3.8 Hz, 2C), 120.3, 119.1, 117.2, 109.4, 96.1, 70.3, 61.6, 48.4, 16.9, 14.1.

### Example 26: ((Methoxycarbonyl)oxy)methyl (E)-3-(1-(3,5-bis(trifluoromethyl)benzyl)-1H-pyrrolo[2,3-b]pyridin-3-yl)-2-cyanoacrylate (PP29)

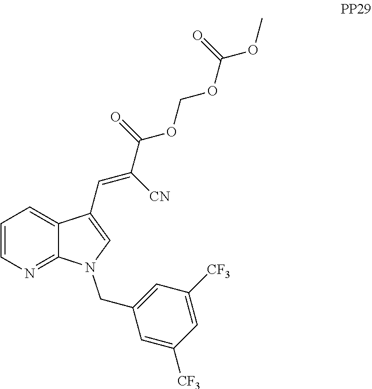 [SMILES: COC(=O)OCOC(=O)/C(C#N)=C/c1cn(Cc2cc(C(F)(F)F)cc(C(F)(F)F)c2)c2ncccc12]

¹H NMR (500 MHz, CDCl₃) δ 8.69 (s, 1H), 8.55 (d, J=0.5 Hz, 1H), 8.49 (dd, J=4.7, 1.5 Hz, 1H), 8.21 (dd, J=8.0, 1.5 Hz, 1H), 7.83 (s, 1H), 7.78 (s, 2H), 7.35 (dd, J=8.0, 4.7 Hz, 1H), 5.96 (s, 2H), 5.70 (s, 2H), 3.86 (s, 3H).

¹³C NMR (125 MHz, CDCl₃) δ 162.0, 154.3, 147.6, 146.5, 145.9, 138.3, 133.6, 132.4 (q, ²J_{C-F}=33.5 Hz, 2C), 127.9 (d, ³J_{C-F}=3.8 Hz), 127.7, 122.9 (q, ¹J_{C-F}=272.9 Hz, 2C), 122.5 (p, ³J_{C-F}=3.8 Hz, 2C), 120.3, 119.3, 116.9, 109.4, 95.3, 82.8, 55.5, 48.4.

### Example 27: 1-Acetyloxyethyl (E)-3-(1-(3,5-bis(trifluoromethyl)benzyl)-1H-pyrrolo[2,3-b]pyridin-3-yl)-2-cyanoacrylate (PP30)

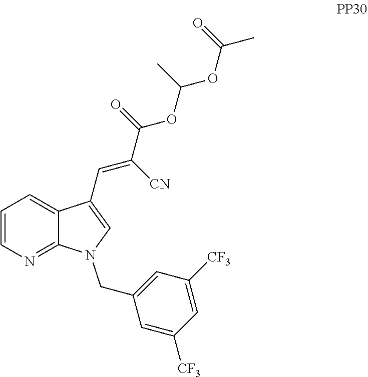 [SMILES: CC(=O)OC(C)OC(=O)/C(C#N)=C/c1cn(Cc2cc(C(F)(F)F)cc(C(F)(F)F)c2)c2ncccc12]

¹H NMR (500 MHz, CDCl₃) δ 8.66 (s, 1H), 8.52 (d, J=0.6 Hz, 1H), 8.47 (dd, J=4.7, 1.5 Hz, 1H), 8.19 (dd, J=8.0, 1.5 Hz, 1H), 7.82 (s, 1H), 7.77 (s, 2H), 7.34 (dd, J=8.0, 4.7 Hz, 1H), 7.04 (q, J=5.5 Hz, 1H), 5.69 (s, 2H), 2.11 (s, 3H), 1.60 (d, J=5.5 Hz, 3H).

¹³C NMR (125 MHz, CDCl₃) δ 168.9, 161.4, 147.6, 146.1, 145.8, 138.4, 133.3, 132.4 (q, ²J_{C-F}=33.6 Hz, 2C), 127.9 (d, ³J_{C-F}=2.9 Hz), 127.7, 122.9 (q, ¹J_{C-F}=272.9 Hz, 2C), 122.5 (p, ³J_{C-F}=3.7 Hz, 2C), 120.3, 119.2, 117.1, 109.4, 95.8, 89.5, 48.4, 20.9, 19.5.

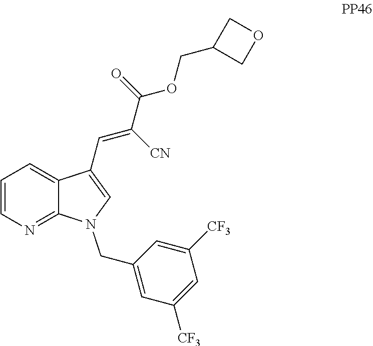 [SMILES: N#C/C(=C\c1cn(Cc2cc(C(F)(F)F)cc(C(F)(F)F)c2)c2ncccc12)C(=O)OCC1COC1]

### Example 28: Oxetan-3-ylmethyl (E)-3-(1-(3,5-bis(trifluoromethyl)benzyl)-1H-pyrrolo[2,3-b]pyridin-3-yl)-2-cyanoacrylate (PP46)

¹H NMR (500 MHz, CDCl₃) δ 8.65 (s, 1H), 8.49 (d, J=0.6 Hz, 1H), 8.46 (dd, J=4.7, 1.5 Hz, 1H), 8.18 (dd, J=7.9, 1.5 Hz, 1H), 7.81 (s, 1H), 7.77 (s, 2H), 7.32 (dd, J=8.1, 4.7 Hz, 1H), 5.68 (s, 2H), 4.85 (dd, J=7.8, 6.4 Hz, 2H), 4.51-4.55 (m, 4H), 3.36-3.44 (m, 1H).

¹³C NMR (125 MHz, CDCl₃) δ 163.3, 147.6, 145.7, 145.5, 138.4, 133.1, 132.3 (q, ²J_{C-F}=33.9 Hz, 2C), 127.9 (d, ³J_{C-F}=4.8 Hz), 127.6, 122.9 (q, ¹J_{C-F}=273.0 Hz, 2C), 122.4 (p, ³J_{C-F}=3.8 Hz, 2C), 120.3, 119.0, 117.3, 109.3, 96.2, 73.9 (2C), 66.9, 48.3, 34.2.

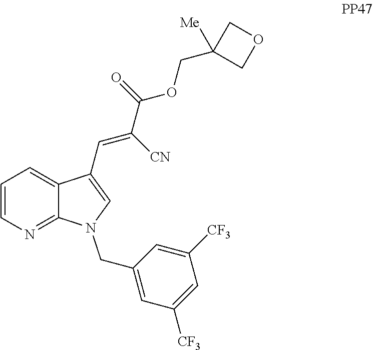 [SMILES: CC1(COC(=O)/C(C#N)=C/c2cn(Cc3cc(C(F)(F)F)cc(C(F)(F)F)c3)c3ncccc23)COC1]

### Example 29: (3-methyloxetan-3-yl)methyl (E)-3-(1-(3,5-bis(trifluoromethyl)benzyl)-1H-pyrrolo[2,3-b]pyridin-3-yl)-2-cyanoacrylate (PP47)

¹H NMR (500 MHz, CDCl₃) δ 8.65 (s, 1H), 8.50 (d, J=0.5 Hz, 1H), 8.46 (dd, J=4.7, 1.5 Hz, 1H), 8.18 (dd, J=7.9, 1.5 Hz, 1H), 7.81 (s, 1H), 7.78 (s, 2H), 7.32 (dd, J=7.9, 4.7 Hz, 1H), 5.69 (s, 2H), 4.56 (d, J=6.1 Hz, 2H), 4.44 (d, J=6.3 Hz, 2H), 4.41 (s, 2H), 1.41 (s, 3H).

¹³C NMR (125 MHz, CDCl₃) δ 163.4, 147.7, 145.8, 145.5, 138.6, 133.2, 132.4 (q, ²J_{C-F}=33.4 Hz, 2C), 128.0 (d, ³J_{C-F}=4.8 Hz), 127.7, 123.0 (q, ¹J_{C-F}=273.0 Hz, 2C), 122.5 (p, ³J_{C-F}=3.8 Hz, 2C), 120.3, 119.1, 117.3, 109.4, 96.3, 79.5 (2C), 70.4, 48.4, 39.5, 21.1.

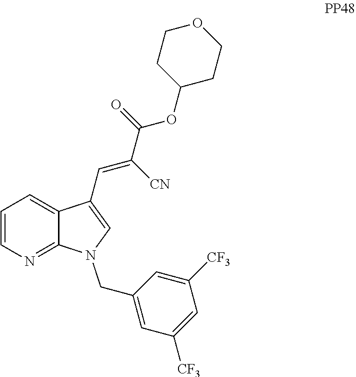 [SMILES: N#C/C(=C\c1cn(Cc2cc(C(F)(F)F)cc(C(F)(F)F)c2)c2ncccc12)C(=O)OC1CCOCC1]

### Example 30: Tetrahydro-2H-pyran-4-yl (E)-3-(1-(3,5-bis(trifluoromethyl)benzyl)-1H-pyrrolo[2,3-b]pyridin-3-yl)-2-cyanoacrylate (PP48)

¹H NMR (500 MHz, CDCl₃) δ 8.65 (s, 1H), 8.49 (d, J=0.6 Hz, 1H), 8.47 (dd, J=4.7, 1.5 Hz, 1H), 8.20 (dd, J=8.1, 1.5 Hz, 1H), 7.82 (s, 1H), 7.77 (s, 2H), 7.33 (dd, J=8.0, 4.7 Hz, 1H), 5.69 (s, 2H), 5.16 (tt, J=8.2, 4.0 Hz, 1H), 3.97 (ddd, J=11.9, 6.0, 4.0 Hz, 2H), 3.60 (ddd, J=11.6, 8.4, 3.2 Hz, 2H), 1.98-2.04 (m, 2H), 1.79-1.86 (m, 2H).

¹³C NMR (125 MHz, CDCl₃) δ 162.5, 147.6, 145.7, 145.1, 138.5, 132.9, 132.4 (q, ²J_{C-F}=33.9 Hz, 2C), 127.9 (d, ³J_{C-F}=4.8 Hz), 127.6, 122.9 (q, ¹J_{C-F}=273.0 Hz, 2C), 122.4 (p, ³J_{C-F}=3.8 Hz, 2C), 120.3, 119.0, 117.4, 109.3, 97.0, 71.1, 65.1 (2C), 48.3, 31.6 (2C).

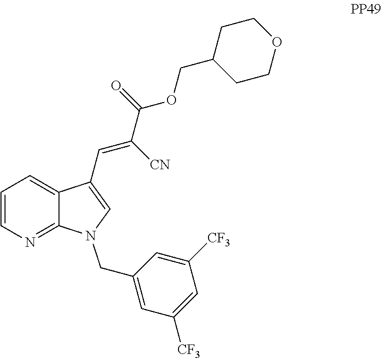 [SMILES: N#C/C(=C\c1cn(Cc2cc(C(F)(F)F)cc(C(F)(F)F)c2)c2ncccc12)C(=O)OCC1CCOCC1]

### (tetrahydro-2H-pyran-4-yl)methyl (E)-3-(1-(3,5-bis(trifluoromethyl)benzyl)-1H-pyrrolo[2,3-b]pyridin-3-yl)-2-cyanoacrylate (PP49)

¹H NMR (500 MHz, CDCl₃) δ 8.64 (s, 1H), 8.49 (d, J=0.8 Hz, 1H), 8.46 (dd, J=4.6, 1.5 Hz, 1H), 8.19 (dd, J=7.9, 1.5 Hz, 1H), 7.81 (s, 1H), 7.77 (s, 2H), 7.32 (dd, J=8.0, 4.7 Hz, 1H), 5.69 (s, 2H), 4.16 (d, J=6.7 Hz, 2H), 4.00 (dd, J=11.2, 3.3 Hz, 2H), 3.41 (td, J=11.9, 2.0 Hz, 2H), 2.00-2.09 (m, 1H), 1.70 (dd, J=12.8, 1.8 Hz, 2H), 1.43 (dd, J=12.1, 4.5 Hz, 2H).

¹³C NMR (125 MHz, CDCl₃) δ 163.2, 147.6, 145.7, 145.2, 138.5, 132.9, 132.4 (q, ²J_{C-F}=33.9 Hz, 2C), 127.9 (d, ³J_{C-F}=4.8 Hz), 127.6, 122.9 (q, ¹J_{C-F}=273.0 Hz, 2C), 122.4 (p, ³J_{C-F}=3.8 Hz, 2C), 120.3, 119.0, 117.4, 109.3, 96.6, 70.2, 67.4 (2C), 48.3, 34.6, 29.4 (2C).

### Testing Methods

### Example 31: Measurement of Esterase Activity of Selected Compounds in Mouse Skin Homogenate

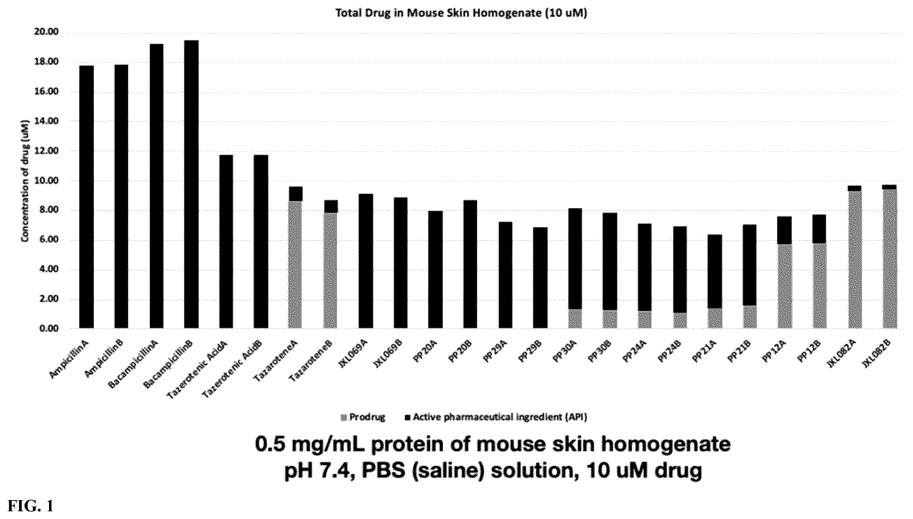

Samples of tazarotene, tazarotenic acid, bacampicillin, ampicillin, JXL069, JXL082, PP12, PP20, PP21, PP24, PP29, PP30, and a reference blank (DMSO) were prepared at a concentration of 10 μM, and incubated with homogenized mouse skin (0.5 mg/mL) in PBS (saline) solution (total volume of 1 mL) for 1 hour at 37° C. The samples were then quenched with UK5099 and analysis was performed using LCMS. Results are shown in FIG. 1.

### Example 32: Measurement of Esterase Activity of Selected Compounds in Minipig Skin Homogenate

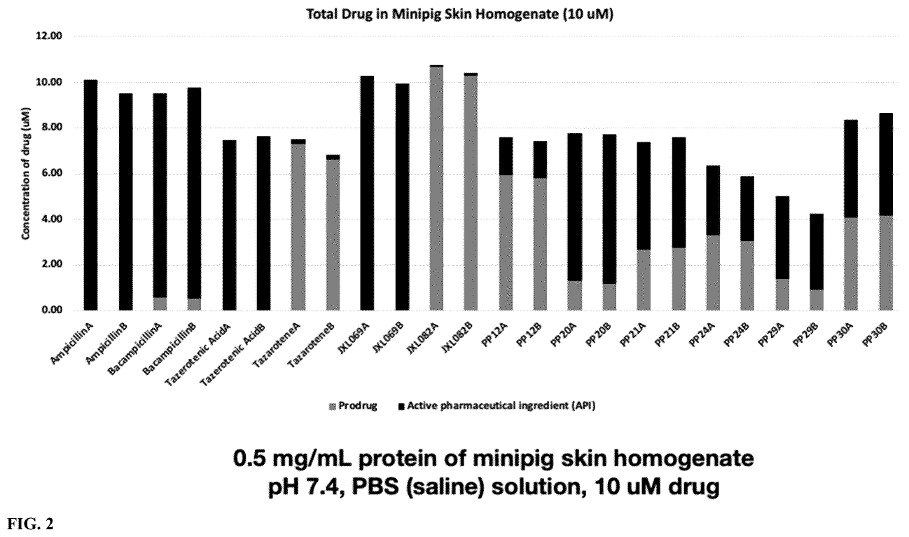

Samples of tazarotene, tazarotenic acid, bacampicillin, ampicillin, JXL069, JXL082, PP12, PP20, PP21, PP24, PP29, PP30, and a reference blank (DMSO) were prepared at a concentration of 10 μM, and incubated with homogenized minipig skin (0.5 mg/mL) in PBS (saline) solution (total volume of 1 mL) for 1 hour at 37° C. The samples were then quenched with UK5099 and analysis was performed using LCMS. Results are shown in FIG. 2.

### Example 33: Measurement of Esterase Activity of Selected Compounds in Human Skin Homogenate

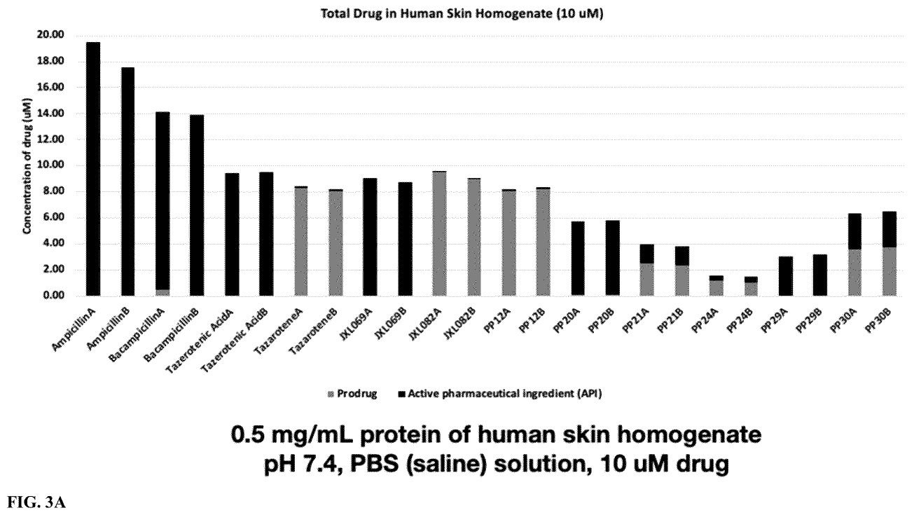

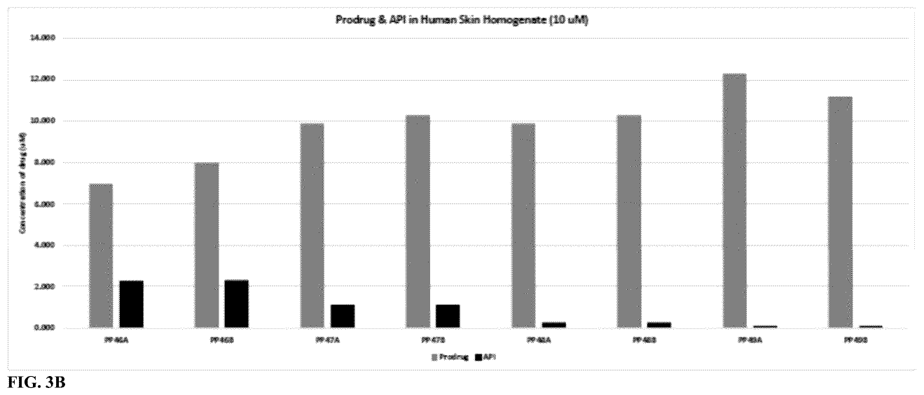

Samples of tazarotene, tazarotenic acid, bacampicillin, ampicillin, JXL069, JXL082, PP12, PP20, PP21, PP24, PP29, PP30, PP46, PP47, PP48, and PP49 and a reference blank (DMSO) were prepared at a concentration of 10 μM, and incubated with homogenized human skin (0.5 mg/mL) in PBS (saline) solution (total volume of 1 mL) for 1 hour at 37° C. The samples were then quenched with UK5099 and analysis was performed using LCMS. Results are shown in FIGS. 3A and 3B.

### Example 34: Ldh Platereader Protocol

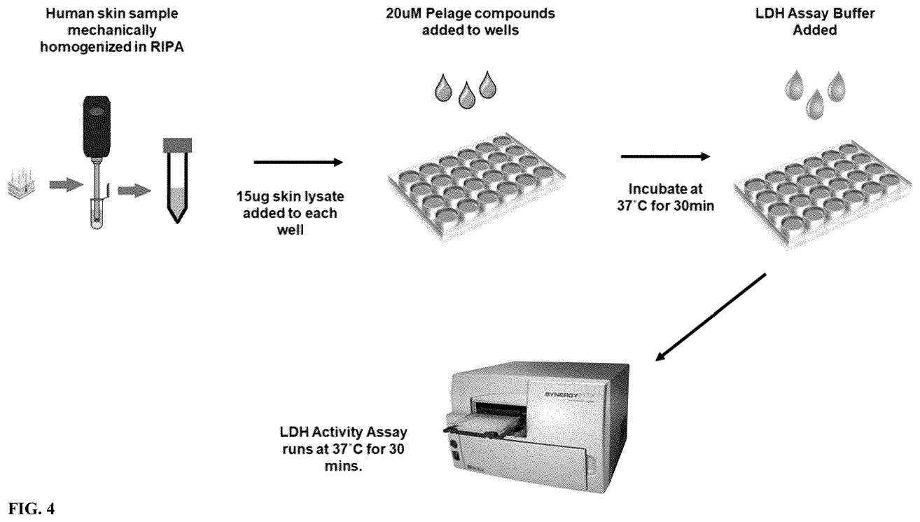

Human skin samples were homogenized with a Benchmark BeadBlaster24R in 500 μl of Pierce RIPA Lysis and Extraction buffer (Thermo Scientific Cat No: 89900), centrifuged at 10,000 g for 15 minutes at 4° C. to remove insoluble material, and the soluble fraction quantified by Pierce BCA assay (Thermo Scientific Cat No: 23225). Lactate dehydrogenase activity was quantified using a Sigma Aldrich Lactate Dehydrogenase Activity Assay Kit (Catalog No: MAK066). Briefly, 10 μg of sample was loaded per well in a 96 well plate along with 20 uM of Pelage compounds and pre-incubated at 37° C. for 30 minutes before addition of Ldh assay master mix. Using a Biotek Synergy HTX multi-mode reader, the plate was incubated at 37° C. and absorbance measurements at 450 nm (A450) were taken every 3 minutes for 30 minutes. The LDH activity of each sample was quantified by calculating the AA450 over time. FIG. 4 shows a schematic for performing the LDH activity assay on human skin cell lysate.

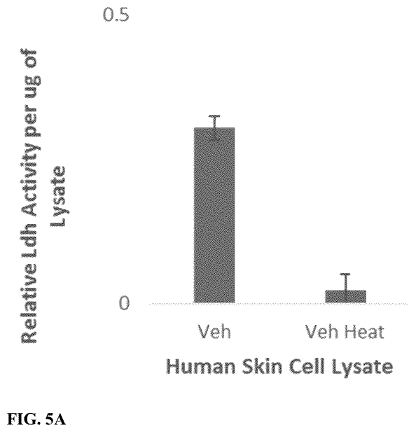

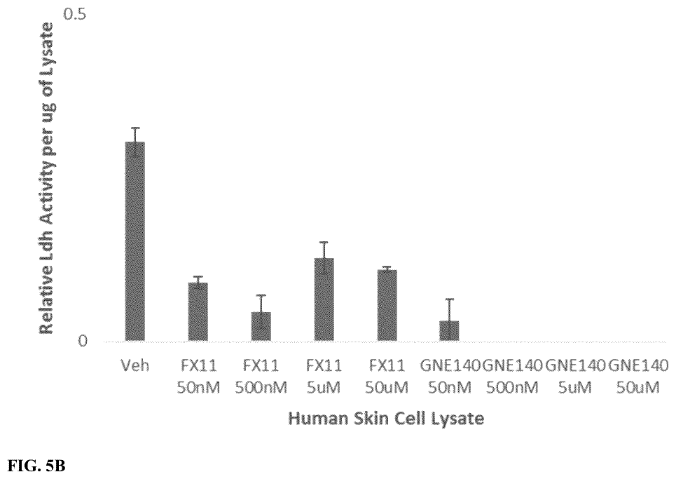

FIG. 5A shows that pretreatment of human skin lysate with high heat kills the LDH activity.

FIG. 5B shows that treatment of human skin lysate with exemplary LDH inhibitors blocks most of the LDH activity, further confirming that the activity readout is the result of LDH activity.

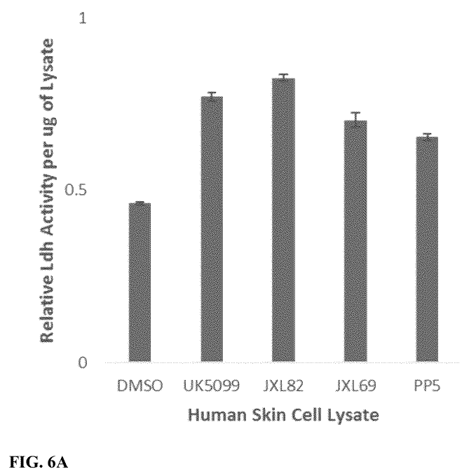

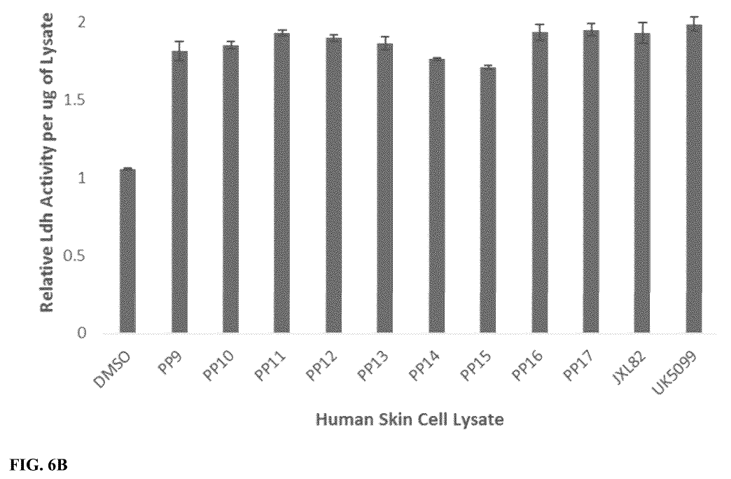

FIGS. 6A and 6B show that the treatment of human skin lysate with exemplary MPC inhibitors results in an increase in LDH activity.

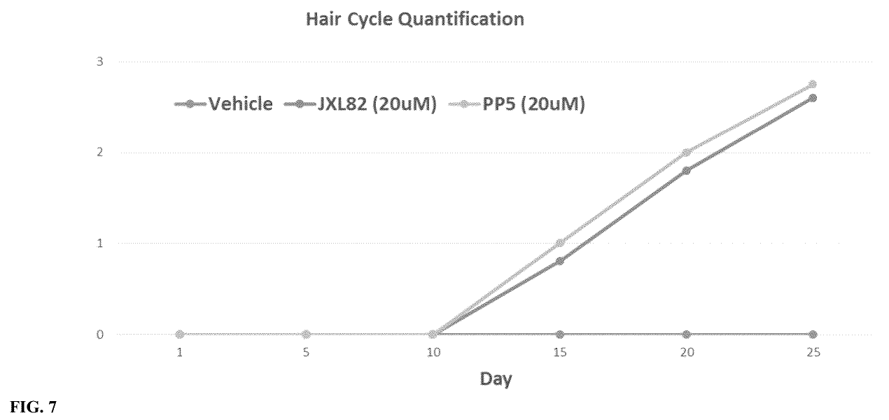

FIG. 7 shows that pretreatment of human skin lysate with a carboxylesterase inhibitor (benzil) prior to incubation with MPC inhibitors blocks the effect of most of the exemplary ester containing MPC inhibitors; however, the pretreatment had no effect on carboxylic containing MPC inhibitors (UK5099, indicated as “UK” in FIG. 7), demonstrating that they act as prodrugs that are only active when converted to carboxylic acids.

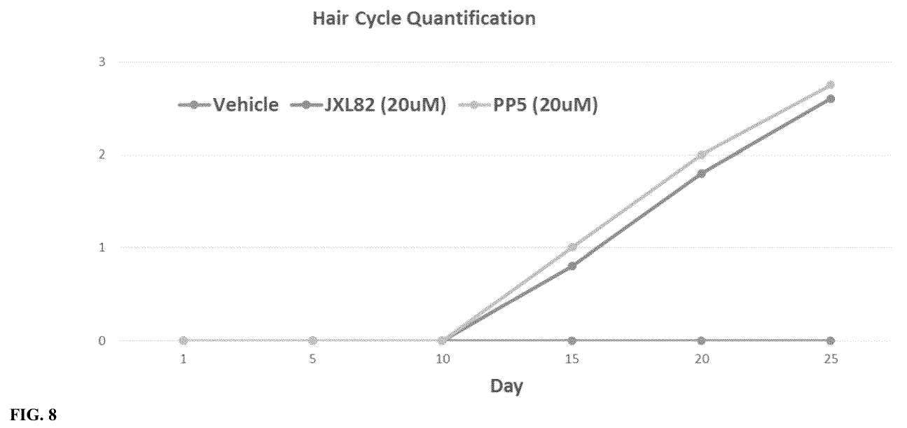

FIG. 8 shows that the MPC inhibitors of the disclosure promote hair growth. Mice were shaved at day 50 when the hair cycle is dormant. Exemplary compounds were applied topically to the shaved area every other day up to day 30. Macroscopic observation led to the quantification of hair cycle staging shown where the two ester-MPC inhibitors accelerated the hair cycle compared to vehicle control.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. All methods described herein may be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A compound represented by a formula: 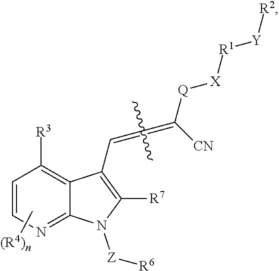
or a pharmaceutically acceptable salt thereof;
wherein Q is —C(═O)—, —C(═S)—, or —S(═O)₂—;
R¹ is —, —S(═O)₂—, an optionally substituted C₁₋₁₂ hydrocarbon group or an optionally substituted heterocycle;
R² is H, an optionally substituted C₁₋₆ alkyl, an optionally substituted carbocycle, or an optionally substituted heterocycle;
R³ and R⁷ are independently H, F, Cl, Br, I, OH, OR^{A}, SH, SR^{A}, NR^{A}R^{B}, CF₃, CN, carboxylic acid, an optionally substituted carboxylic ester, or an optionally substituted C₁₋₆ alkyl;
each R⁴ is independently H, F, Cl, Br, I, OH, O⁺³¹, OR^{A}, SH, SR^{A}, NR^{A}R^{B}, CF₃, CN, carboxylic acid, an optionally substituted carboxylic ester, or an optionally substituted C₁₋₆ alkyl;
R^{A} and R^{B} are independently H or optionally substituted C₁₋₆ hydrocarbon group;
n is 0, 1, or 2;
X is O, S, NR⁵, or N⁺R⁵R⁸, wherein R⁵ and R⁸ are independently H, C₁₋₆ alkyl, an optionally substituted carbocycle, or an optionally substituted heterocycle, and the N, R⁵ and R¹, or the N, R⁵ and R⁸, may together form an optionally substituted heterocyclic ring;
Y is —, —O—, 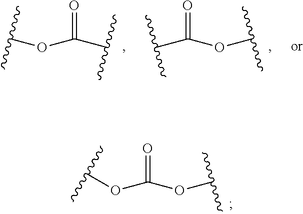 [SMILES: *OC(*)=O ; *OC(*)=O ; *OC(=O)O*]
Z is —, an optionally substituted C₁₋₁₂ hydrocarbon group, or optionally substituted heterocycle;
R⁶ is H, an optionally substituted C₁₋₁₂ hydrocarbon group, or optionally substituted heterocycle; and
the wavy line across the C═C bond represents an E or Z olefin.

2. The compound of claim 1, wherein X is O.

3. The compound of claim 2, further represented by a formula: 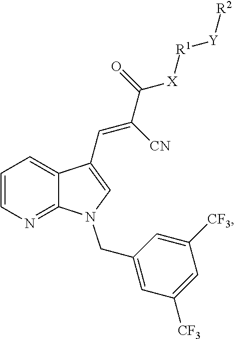
or a pharmaceutically acceptable salt thereof.

4. The compound of claim 3, wherein R¹ is —, —CH₂—, an optionally substituted C₃₋₁₂ hydrocarbon group, or an optionally substituted heterocycle having a carbon atom directly attached to X.

5. The compound of claim 2, further represented by a formula: 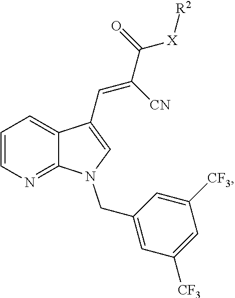
or a pharmaceutically acceptable salt thereof;
wherein R² is CH₃ or C₃₋₁₂ alkyl.

6. The compound of claim 1, wherein X is NR⁵ or N⁺R⁵R⁸.

7. (canceled)

8. (canceled)

9. The compound of claim 1, wherein R¹ is an optionally substituted C₁₋₁₂ alkyl, optionally substituted heterocycle, or optionally substituted benzyl.

10-17. (canceled)

18. The compound of claim 1, wherein R¹ is an optionally substituted oxetane, optionally substituted tetrahydrofuran, optionally substituted dihydrofuran, optionally substituted furan, optionally substituted furanone, optionally substituted tetrahydropyran, optionally substituted dihydropyran, optionally substituted pyran, optionally substituted tetrahydropyrone, optionally substituted dihydropyrone, optionally substituted thietane, optionally substituted tetrahydrothiophene, optionally substituted dihydrothiophene, optionally substituted thiophene, optionally substituted azetidine, optionally substituted pyrrolidine, optionally substituted pyrroline, optionally substituted pyrrole, optionally substituted pineridine, optionally substituted pyridine, optionally substituted oxazole, optionally substituted isoxazole, optionally substituted thiazole, optionally substituted isothiazole, optionally substituted pyrazolidine, optionally substituted imidazolidine, optionally substituted pyrazole, optionally substituted imidazole, optionally substituted tetrazole, or optionally substituted sulfolane.

19-48. (canceled)

49. The compound of claim 1, wherein Y is —, —O—,

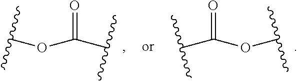 [SMILES: *OC(*)=O ; *OC(*)=O]

50-53. (canceled)

54. The compound of claim 1, wherein R² is H or C₁₋₁₂ alkyl.

55. (canceled)

56. (canceled)

57. The compound of claim 1, wherein R² is optionally substituted heterocycle.

58. The compound of claim 1, wherein R² is optionally substituted aryl.

59. (canceled)

60. The compound of claim 1, wherein R² is optionally substituted benzyl.

61. The compound of claim 1, wherein R³ is H or F.

62. (canceled)

63. (canceled)

64. The compound of claim 1, wherein Z is optionally substituted C₁₋₁₂ alkyl.

65-80. (canceled)

81. The compound of claim 1, wherein R⁶ is optionally substituted aryl.

82. (canceled)

83. (canceled)

84. The compound of claim 1, wherein R^{b} is

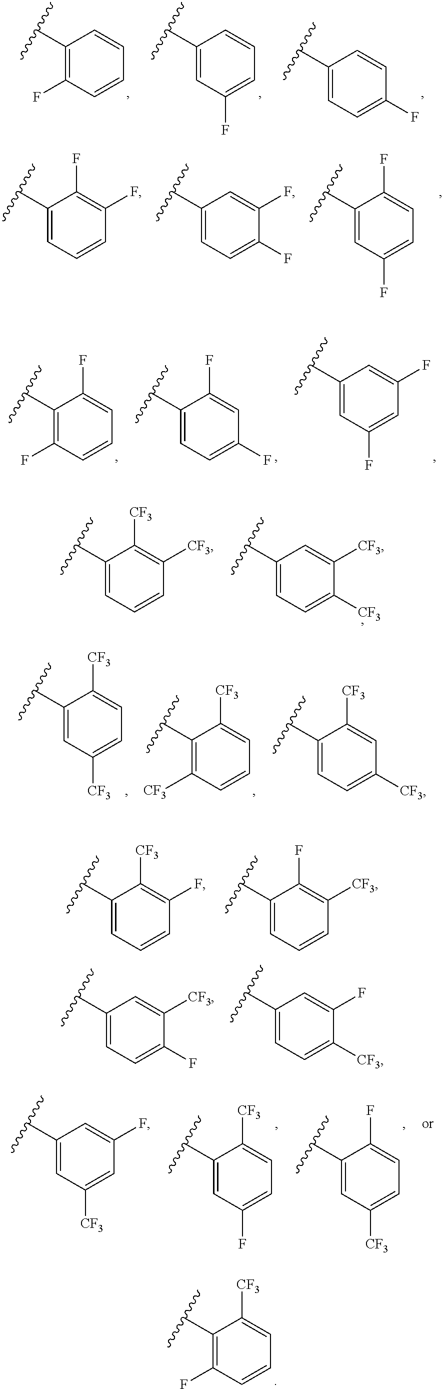

85-89. (canceled)

90. A compound, which is:

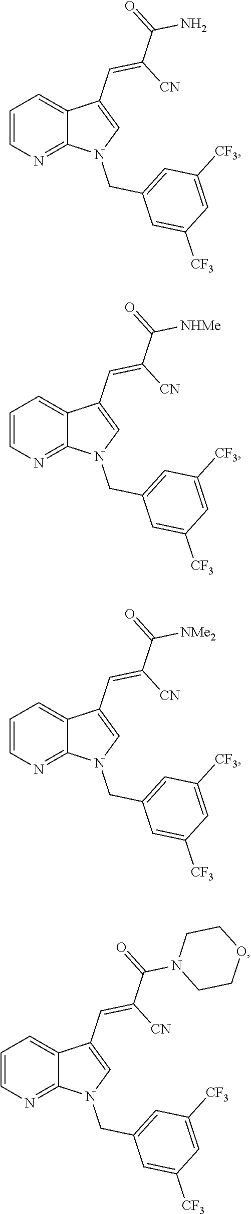 [SMILES: N#C/C(=C\c1cn(Cc2cc(C(F)(F)F)cc(C(F)(F)F)c2)c2ncccc12)C(N)=O ; CNC(=O)/C(C#N)=C/c1cn(Cc2cc(C(F)(F)F)cc(C(F)(F)F)c2)c2ncccc12 ; CN(C)C(=O)/C(C#N)=C/c1cn(Cc2cc(C(F)(F)F)cc(C(F)(F)F)c2)c2ncccc12 ; N#C/C(=C\c1cn(Cc2cc(C(F)(F)F)cc(C(F)(F)F)c2)c2ncccc12)C(=O)N1CCOCC1]

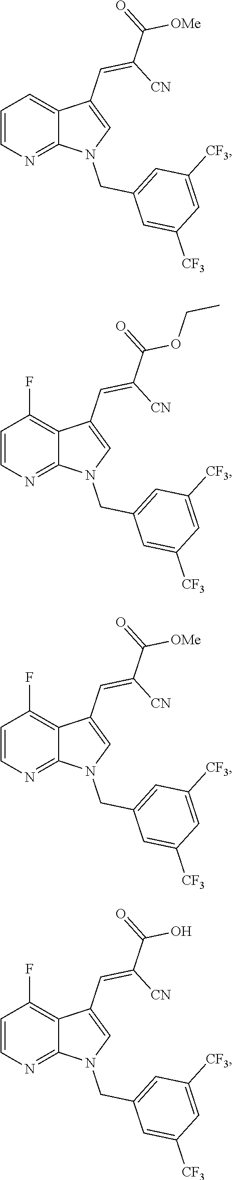 [SMILES: COC(=O)/C(C#N)=C/c1cn(Cc2cc(C(F)(F)F)cc(C(F)(F)F)c2)c2ncccc12 ; CCOC(=O)/C(C#N)=C/c1cn(Cc2cc(C(F)(F)F)cc(C(F)(F)F)c2)c2nccc(F)c12 ; COC(=O)/C(C#N)=C/c1cn(Cc2cc(C(F)(F)F)cc(C(F)(F)F)c2)c2nccc(F)c12 ; N#C/C(=C\c1cn(Cc2cc(C(F)(F)F)cc(C(F)(F)F)c2)c2nccc(F)c12)C(=O)O]

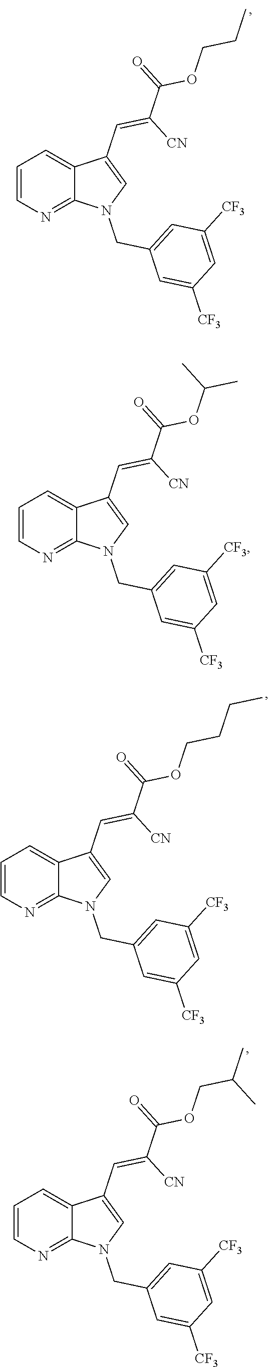 [SMILES: CCCOC(=O)/C(C#N)=C/c1cn(Cc2cc(C(F)(F)F)cc(C(F)(F)F)c2)c2ncccc12 ; CC(C)OC(=O)/C(C#N)=C/c1cn(Cc2cc(C(F)(F)F)cc(C(F)(F)F)c2)c2ncccc12 ; CCCCOC(=O)/C(C#N)=C/c1cn(Cc2cc(C(F)(F)F)cc(C(F)(F)F)c2)c2ncccc12 ; CC(C)COC(=O)/C(C#N)=C/c1cn(Cc2cc(C(F)(F)F)cc(C(F)(F)F)c2)c2ncccc12]

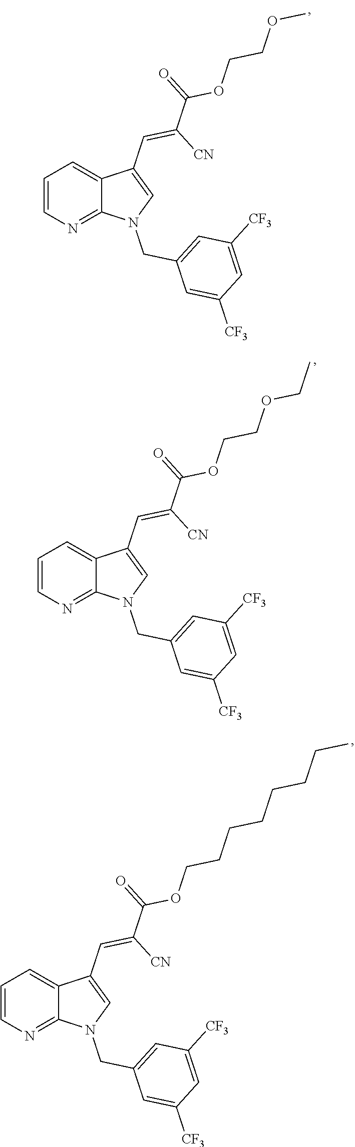 [SMILES: COCCOC(=O)/C(C#N)=C/c1cn(Cc2cc(C(F)(F)F)cc(C(F)(F)F)c2)c2ncccc12 ; CCOCCOC(=O)/C(C#N)=C/c1cn(Cc2cc(C(F)(F)F)cc(C(F)(F)F)c2)c2ncccc12 ; CCCCCCCCOC(=O)/C(C#N)=C/c1cn(Cc2cc(C(F)(F)F)cc(C(F)(F)F)c2)c2ncccc12]

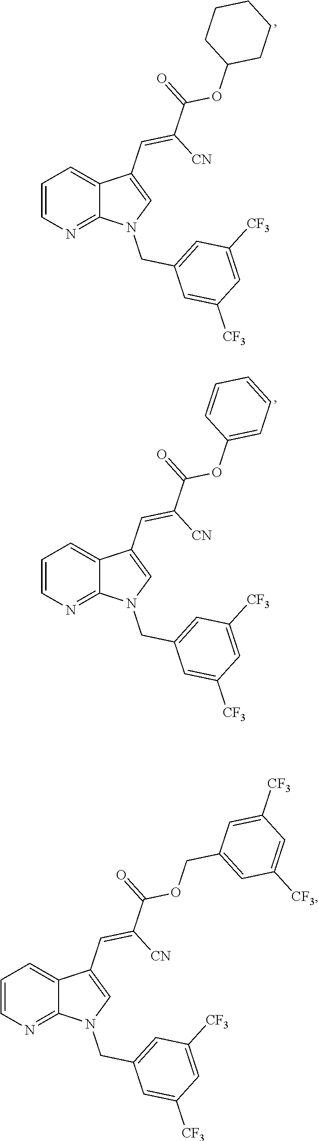

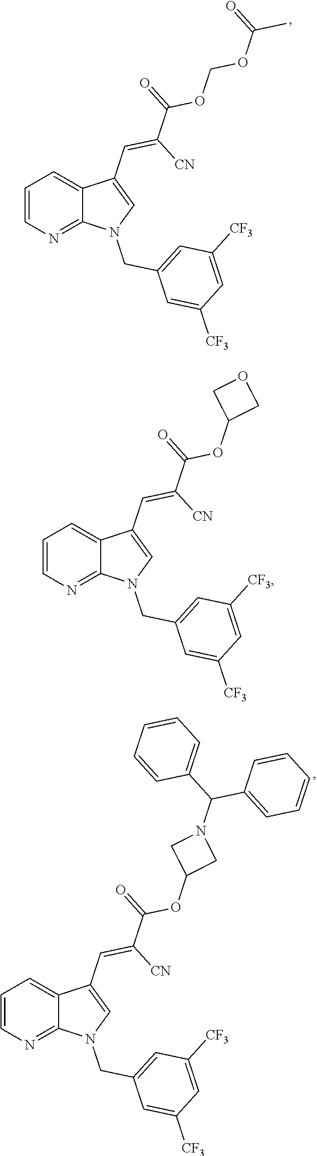 [SMILES: CC(=O)OCOC(=O)/C(C#N)=C/c1cn(Cc2cc(C(F)(F)F)cc(C(F)(F)F)c2)c2ncccc12 ; N#C/C(=C\c1cn(Cc2cc(C(F)(F)F)cc(C(F)(F)F)c2)c2ncccc12)C(=O)OC1COC1 ; N#C/C(=C\c1cn(Cc2cc(C(F)(F)F)cc(C(F)(F)F)c2)c2ncccc12)C(=O)OC1CN(C(c2ccccc2)c2ccccc2)C1]

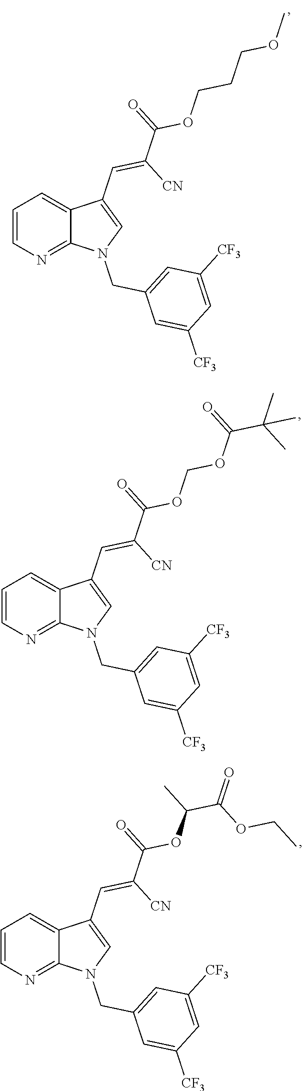

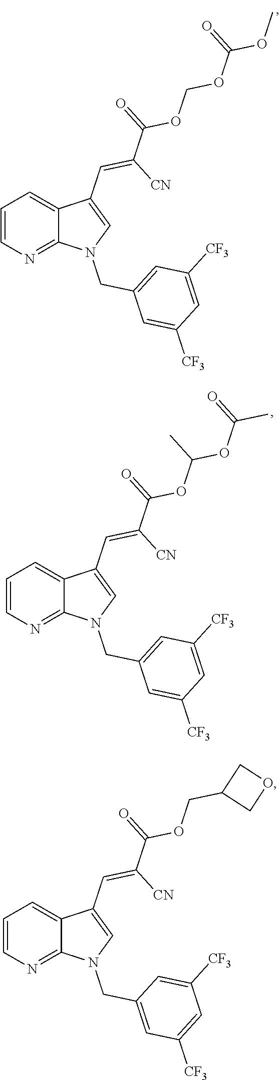

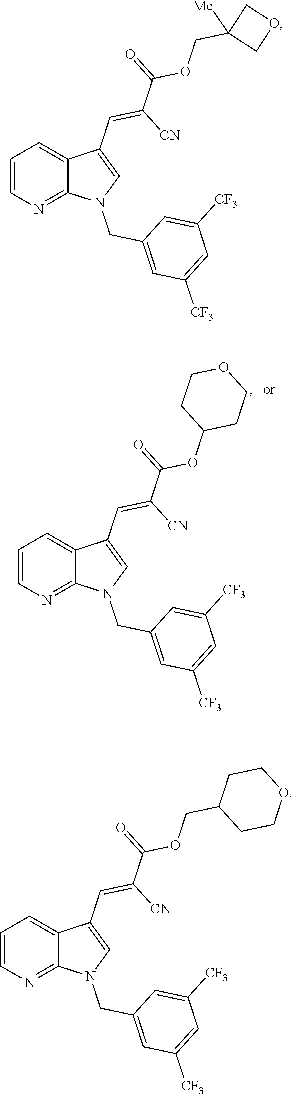 [SMILES: CC1(COC(=O)/C(C#N)=C/c2cn(Cc3cc(C(F)(F)F)cc(C(F)(F)F)c3)c3ncccc23)COC1 ; N#C/C(=C\c1cn(Cc2cc(C(F)(F)F)cc(C(F)(F)F)c2)c2ncccc12)C(=O)OC1CCOCC1 ; N#C/C(=C\c1cn(Cc2cc(C(F)(F)F)cc(C(F)(F)F)c2)c2ncccc12)C(=O)OCC1CCOCC1]

91. (canceled)

92. (canceled)

93. A pharmaceutical composition comprising a compound of claim 1 and a pharmaceutically acceptable excipient.

94-97. (canceled)

98. A method of treating a disorder affecting hair growth in a subject comprising administering a compound of claim 1 to the subject.

99. (canceled)
